(19) United States
(12) Patent Application Publication
(10) Pub. No.: US 2022/0002429 A1
(43) Pub. Date: Jan. 6, 2022

(54) TUMOR CELL AGGREGATION INHIBITORS' FOR TREATING CANCER

(71) Applicant: Northwestern University, Evanston, IL (US)

(72) Inventors: Huiping Liu, Chicago, IL (US); Xia Liu, Chicago, IL (US); Valery Adorno-Cruz, Chicago, IL (US); Rokana Taftaf, Chicago, IL (US)

(21) Appl. No.: 17/309,115

(22) PCT Filed: Oct. 24, 2019
(86) PCT No.: PCT/US2019/057917

§ 371 Date: Apr. 26, 2021

Related U.S. Application Data
(60) Provisional application No. 62/749,772, filed on Oct. 24, 2018.

(51) Int. Cl. C07K 16/28; A61K 31/519; A61P 35/04

(52) U.S. Cl. CPC C07K 16/2884; A61K 31/519; C07K 2317/76; C07K 16/2863; C07K 16/2821; A61P 35/04

(57) ABSTRACT

Disclosed are methods for treating cancer in a subject. The methods typically include administering to the subject a therapeutic agent that inhibits aggregation of tumor cells.

### CROSS-REFERENCE TO RELATED PATENT APPLICATIONS

This application claims the benefit of priority under 35 U.S.C. § 119(e) to U.S. Provisional Patent Application No. 62/749,772, filed on Oct. 24, 2018, the content of which is incorporated herein by reference in its entirety.

### STATEMENT REGARDING FEDERALLY SPONSORED RESEARCH OR DEVELOPMENT

This invention was made with government support under W81WWH-16-1-0021 awarded by the Department of Defense and CA160638 awarded by the National Institutes of Health. The government has certain rights in the invention.

### BACKGROUND

The field of the invention relates to methods for treating and diagnosing cancers characterized by tumor cell aggregation. In particular, the field of the invention relates to methods for inhibiting tumor cell aggregation that is associated with metastasis in order to treat cancer.

Metastasis is the killer of 90% of patients with solid tumors. As such, metastasis remains as the major cause of cancer mortality and it demands a better understanding for more effective treatments. In order for tumor cells to metastasize, they must overcome several barriers. One of the first a few steps are invasion and intravasation from the primary tumor in order to circulate through the peripheral vasculature. Circulating tumor cells (CTCs) are associated with a poor prognosis. In addition to the dogma of single cell-mediated dissemination, we recently demonstrated that clustered CTCs are more tumorigenic and metastatic than single CTCs with advantages of enhanced regenerative power or stem cell properties (stemness)¹. However, there is no existing therapeutics targeting CTC clusters to our knowledge. Here, we have identified new molecular targets of tumor clusters that are responsible for mediating cancer metastasis.

The present inventors' work has demonstrated that CD44⁺ breast cancer stem cells (CSCs) are largely responsible for metastasis and therapy resistance. These CSCs form circulating tumor cell (CTC) clusters to seed cancer metastases and are associated with poor survival of patients with breast cancer at advanced stage. There is an unmet need in the field to target CD44⁺ CSCs, block their aggregation and cluster formation, and thereby prevent and treat metastasis. The inventors have found that depletion of CD44 effectively prevented tumor cell aggregation and decreased the levels of protein activated kinase 2 (PAK2). The inventors have shown that intercellular CD44-CD44 homophilic interactions direct multicellular aggregation and that the N-terminal domain of CD44 is required for these intercellular CD44-CD44 homophilic interactions. Furthermore, these intercellular CD44-CD44 homophilic interactions initiate CD44-PAK2 interactions which further result in signaling by focal adhesion kinase (FAK). The inventors also have determined that CD44 promotes epidermal growth factor receptor (EGFR) activity to enhance aggregation and cluster formation.

The inventors' studies highlight that CD44⁺ CTC clusters can serve as novel therapeutic targets for inhibiting or preventing polyclonal metastasis. Targeting strategies may include administering therapeutic agents such as anti-CD44 antibodies or antigen-binding fragment thereof, anti-EGFR antibody antibodies or antigen-binding fragment thereof, and/or PAK2 inhibitors (PAK2i), where the therapeutic agents functionally block CD44+ CSC cluster formation and polyclonal lung metastases.

The epidermal growth factor receptor (EGFR) is a tyrosine kinase that has been known to be involved in several cancers by promoting its growth, differentiation and migration. The present inventors also have shown that EGFR contributes to the formation of this cell aggregation in a synergy with CD44. The inventors that found an EGFR monoclonal antibody (anti-EGFR, clone LA1, Millipore) effectively blocks clustering in vitro and reduces lung metastasis. Furthermore, the inventors present miR-30c as a potential therapeutic to disrupt CD44 and EGFR mediated clustering.

Finally, the inventors found that intercellular adhesion molecule 1 (ICAM1) is highly enriched in the lung metastatic cells and circulating tumor cell clusters and that ICAM1 directs intercellular homophilic interactions between tumor-tumor cells as well as tumor-endothelial cells. The inventors determined that ICAM1 knockdown abolishes the tumor cell clustering and lung colonization of breast cancer cells. In addition, the inventors found that two anti-ICAM1 neutralizing antibodies can block tumor clustering as well as transendothelial migration of breast cancer cells during metastasis.

The proposed therapies may be administered for breast cancer early stage treatment to shrink primary tumors and prevent metastasis for extended survival. The proposed therapies also may be administered for breast cancer late stage treatment to kill CSCs in primary tumors, circulation and secondary organs, thereby prolong patient survival.

### SUMMARY

Disclosed are methods for treating and diagnosing cancers characterized by tumor cell aggregation.

The disclosed methods of treatment may include methods for inhibiting tumor cell aggregation that is associated with metastasis in order to treat cancer. Cancers that may be treated by the disclosed methods include cancers characterized by circulating tumor cells (CTCs), including CTCs that express one or more of CD44, PAK2, EGFR, and ICAM1.

The disclosed methods of treatment may include, but are not limited to, methods of treating cancers such as breast cancer. Suitable breast cancers treated by the disclosed methods may include estrogen receptor (ER)-negative breast cancer, receptor (PR)-negative breast cancer, and epidermal growth factor receptor 2 (HER2)-negative breast cancer, for example, where the breast cancer is triple negative breast cancer (TNBC).

The disclosed treatment methods typically include administering to a subject need thereof a therapeutic agent that inhibits tumor cell aggregation. Suitable therapeutic agents for use in the disclosed methods may include, but are not limited to, therapeutic agents that inhibit the expression and/or biological activity of one or more of CD44, PAK2, EGFR, and ICAM1. Suitable therapeutic agents may include antibodies against one or more of CD44, PAK2, EGFR, and ICAM1 or antigen-binding fragments thereof. Suitable therapeutic agents may include kinase inhibitors, for example, kinase inhibitors which inhibit the kinase activity of PAK2 or EGRF.

Also disclosed herein are diagnostic methods. The disclosed methods may include detecting expression of one or more of CD44, PAK2, EGFR, and ICAM1 in circulating tumor cells of a subject having cancer such as breast cancer. In the disclosed diagnostic methods, the subject therein diagnosed may be identified as having a high risk for developing metastatic breast cancer. The disclosed diagnostic methods further may include a treatment step. For example, the diagnostic methods may include a step of administering to the subject a therapeutic agent that inhibits aggregation of tumor cells such as a therapeutic agent that inhibits the biological activity or expression of one or more of CD44, PAK2, EGFR, ICAM1.

### BRIEF DESCRIPTION OF THE FIGURES

FIG. 1. Tumor cell clusters arise from cellular aggregation. A. H & E staining images of CTC clusters (arrows) within the vasculature of the lung metastasis sections of TNBC patient CW1 (left panel) and a TN1 PDX mouse (right panel). Scale bars=10 μm. B. IHC staining with a TN PDX breast tumor section for cytokeratin (CK) showing clustered tumor cells within the vasculature (a lower magnification image is in FIG. 8B). Scale bar=10 μm. C. Frequencies of IHC-detected vascular CTC clusters (% of all CTC events) within breast tumor and distant metastasis sections of seven patients (n=9 human tissues) and seven PDX models (n=28 mouse tissues) (listed in Table 1). T-test p=0.1115 (NS). D. Human CTC clusters in the peripheral blood of patients with metastatic breast cancer, negative for CD45 and positive for pan-CK and nuclear DNA (DAPI), detected via EpCAM-based CellSearch platform. Scale bars=10 μm. E. Fluorescence images of TN1 PDX tumor cell clusters within the peripheral blood and the lungs of NOD/SCID mice. Top panel: blood CTC cluster (tdTomato⁺) from L2T PDX-bearing mice (Hoechst). Bottom panel: 3D stack image of a dual-color lung colony with one L2G (eGFP⁺) cell and one L2T (tdTomato⁺) cell derived from mixed-color implants as shown in FIG. 9A. Scale bars=10 μm. F. Frequencies of blood CTC clusters (% of all CTC events) isolated from seven patients with metastatic breast cancer (n=7) and mice with four PDX models (n=7 mice) (Table 2). T-test p=0.533 (NS). G. Intravital images of TN1 PDX breast tumor cell cluster formation via cell aggregation during migration, showing individually migrating eGFP⁺ tumor cells approaching and aggregating with other tumor cells and moving around dynamically. Arrows at 24′ and 30′ show the cumulative paths of cells 1, 2, and 3. Dextran+ vessels, and second harmonic generation (collagen I fibers) are indicated. Scale bar=10 μm. H. Intravital images of single-cell intravasation of eGFP⁺ MDA-MB-231 tumor cells following cluster formation in a primary tumor. Stationary tumor cell 1 is joined by individually migrating cells 2 & 3 to form a cluster. Cell number 2 sequentially leaves the cluster and intravasates between the frames at 18′ and 20′. Tumor cells and vasculature are indicated. Scale bar=10 μm. I. Intravital images of eGFP⁺ PyMT breast tumor cells in MacBlue Rag^{-/-} mice, circulating as single cells (T1 and T5) and as groups of cells (T2, T3, and T4) in close physical proximity to each other. Tumor cells are briefly observed as they rapidly pass through the imaging field due to blood flow. Tumor cells, CTCs, vasculature, and macrophages are indicated. Scale bar=10 μm. J. Patient-derived CTC line BRX50 cells form clusters within one to two hours of suspension culture. Scale bar=50 μm. K. Cluster formation within the lung vasculature imaged ex vivo at 2 h after tail vein infusion of eGFP+ and tdTomato⁺ MDA-MB-231 cells at 1:1 ratio, either mixed co-infusion (0 min apart), or separate infusions of tdTomato⁺ cells first and then eGFP⁺ cells lagged at 5 min, 10 min, and 2 h. Ex vivo lung fluorescence images were taken 2 h post-infusion of eGFP⁺ cells. Scale bars=50 μm. L. Quantitative proportions of single-color and mixed-color clusters (lung colonies) from the four groups in panel K. The experiments were repeated three times (n=3) with counts of at least five images per mouse. T-test, ***p<0.001.

FIG. 2. Tumor cell clusters with increased tumorigenesis, metastasis, and CD44 expression. A. Representative bioluminescence images of single cells (S) and clusters (C) of TN1 PDX tumor cells in parallel during tumorigenic monitoring upon orthotopic implantation (1,000 cells per mammary fat pad), on days 0 (D0) and 18 (D18). B. Quantitative bioluminescence signals (total flux, p/s) (left panel) and fold change (right panel) of tumorigenesis mediated by TN1 tumor cells in singles and clusters during the 18-day monitoring in panel A (n=5). T-test **p<0.01. C. Representative bioluminescence images of single cells (S) and clusters (C) of TN2 PDX tumor cells in parallel during tumorigenic monitoring upon orthotopic implantation (5,000 cells per mammary fat pad), on day 0 (D0) and day 24 (D24). D. Quantitative bioluminescence signals (total flux, p/s) (left panel) and fold change (right panel) of tumorigenesis mediated by TN2 tumor cells in singles and clusters during the 24-day monitoring in C (n=8). T-test *p<0.05, **p<0.01, ***p<0.001. E. Images of mammospheres derived from single and clustered tumor cells of TN2 PDXs. Scale bars=50 μm. F. Quantitated bar graph of mammospheres derived from single cells and clusters. N=6 biological replicates. ***p=0.0008. G. BLI images of lung colonization mediated by single cells and clusters of L2T-labeled TN1 PDX cells (5×10⁵) at day 0 (D0) and weeks 1, 2 and 8 (Wk1, 2, 8) after tail vein infusion. H. Quantitated BLI signal (total flux, p/s) of the TN1 single cell- and cluster-mediated lung metastases described in panel G (n=5). T-test *p=0.012, ***p=0.00012. I. CD44 and CD31 IHC staining of the TN1 PDX-bearing mouse lung sections (slide 228) showing a CD44⁺ CTC cluster within CD31⁺ vasculature. Scale bar=25 μm. J. Proportion of CD44⁺ CTCs in the events of single and cluster CTCs counted within the in situ vasculature of human breast tumors (n=5) and metastases (n=3), and PDX lung metastases (n=3) (Table 3). T-test ****p<0.0001.

FIG. 3. CD44 directs polyclonal tumor cell aggregation. A. Time lapse aggregation images at 0, 4, 8, and 24 h incubation with PDX-derived 1:1 mixtures of tdTomato+ and eGFP+ primary tumor cells. Left column: sorted CD44⁺; middle column, CD44⁻; right column, mixed CD44⁺/CD44⁻ cells in two colors. B. Number of viable CD44⁺ versus CD44⁻ cells at the time points of 12 and 24 h aggregation (T-test p=0.6, n=4). C. Immunoblot of CD44 and β-actin (loading control) in TN1 PDX tumor cells upon transfection of the scrambled siRNA control (siCon) or siCD44, which caused a depletion of the dominant variant CD44 (CD44v, molecular weight 120˜160 kDa) and the marginal standard CD44 (CD44s, molecular weight ˜80 kDa). D. CD44 immunoblot showing siCD44-mediated knockdown of the exclusive CD44s in MDA-MB-231 cells compared to the control (siCon). E. CD44 immunoblot of three MDA-MB-231 batches of populations upon CRISPR/Cas9-based CD44 knockout (KO) with gRNA targeting exon 2. F. Snapshot images showing reduced aggregation of L2G-labeled TN1 PDX tumor cells at 72-h clustering time point after siCD44 and siCon transfections, measured via IncuCyte imaging system. G. Quantitative curves of the cluster size (left panel) and number (right panel) of TN1 cells measured via IncuCyte time-lapse imaging (n=3 biological replicates, MANOVA ***p<0.001). H. Snapshot images taken at 0 and 60 min of MDA-MB-231 suspension cell aggregation onto poly-hydroxyethyl methacrylate-treated plates, at 48 h after siCD44 and siCon transfections. Scale bars=50 μm. I. Cluster counts of MDA-MB-231 cells at 60 min aggregation. The data were from at least five images for each group per experiment. The experiments were repeated three times (n=3). T-test *p<0.05, **p<0.01, ***p<0.001. J. Cluster images of CD44 WT and KO (via CRISPR/Cas9) MDA-MB-231 cells at the 24 h aggregation time-point. Scale bars=150 μm. K. Quantitative counts of clustered MDA-MB-231 cells with a cluster size >20 cells show a significant, dramatic reduction in CD44 KO cells (n=5, T-test ***p<0.001).

FIG. 4. CD44 depletion blocks tumor cell aggregation and lung metastasis in vivo. A. Bioluminescence images of lung colonization of the siRNA control (siCon) and siCD44-transfected TN1 tumor cells on days 0 (D0) and 1 (D1) and weeks 1 (Wk1) and 4 (Wk4) post-tail vein infusion. 5×10⁵ cells were injected into mice at 36 h after the initial transfection. B. Quantitative bioluminescence signal curves (total flux, p/s) of the TN1 PDXs (siCon and siCD44) as measured in A, n=5 mice per group. T-test ***p<0.001. C. Bioluminescence images of lung colonization of the siRNA control (siCon) and siCD44-transfected TN2 tumor cells on days 0 (D0) and 1 (D1) and week 1 (Wk1) post-tail vein infusion. 5×10⁵ cells were injected to mice at 36 h after the initial transfection. D. Quantitative bioluminescence signal curves (total flux, p/s) of the TN2 PDXs (siCon and siCD44) as measured in C, n=5 mice per group. T-test **p<0.01. E. Bioluminescence images of lung colonization of L2T-labeled CD44 WT and KO MDA-MB-231 tumor cells on day 0 (D0) and weeks 1 (Wkl), 3 (Wk3), and 5 (Wk5) post-tail vein infusion. F. Quantitative analyses of the lung bioluminescence signals of CD44 WT and KO cells imaged in E (n=4). T-test *p<0.05, ****p<0.0001. G. Fluorescence images of the lungs at 2 and 24 h and 5 weeks post-tail vein infusion of mixed eGFP⁺CD44KO and tdTomato⁺CD44WT tumor cells (1:1 ratio). Three columns represent the images from the tdTomato channel, the eGFP channel, or the merged channels, respectively. Scale bars=50 μm for the images taken at 2 and 24 h, and 125 μm for the images taken at week 5. H. Counts of single (solid bar) or clustered tumor cells (checked bar) of CD44 WT and KO cells in the lung images at 2 and 24 h post-tail vein injections in E. At least five lung images were taken for each mouse (n=3 mice). I. Tumorigenesis results of PDX-derived CD44⁺ WT and CD44⁻ KO cells implanted into the mammary fat pads (2^{nd} and 4^{th}), from 1,000 to 100,000 cells per injection. *T-test p=0.02 between WT/KO implantations of 1,000 cells. J. Top: Depiction of orthotopic implantation of CD44 WT (eGFP⁺) and KO (tdTomato⁺) tumor cells (100,000 cell per injection) separately into the left and right mammary fat pads of each NOD/SCID mouse. Bottom: images of breast tumors derived from the above implantations at harvest (3 weeks). K. Comparison of tumor weight of CD44 WT (eGFP+) and KO (tdTomato⁺) tumors derived from 100,000 cell injections in J (n=6 mice). T-test ***p<0.001. L. Fluorescence images, from the channels of tdTomato (left), eGFP (middle), and merged (right), of the dissected lungs with spontaneous metastases of CD44 WT (eGFP⁺) and KO (tdTomato⁺) tumors, at 3 weeks post-orthotopic implantation of these cells into the left and right 4^{th} mammary fat pads, respectively (as shown in J). Scale bars=125 μm. M&N. Comparison of lung colonies (M) and normalized lung metastases (colony # per gram of tumor weight) (N) following implantations of CD44 WT and KO cells. T-test ****p<0.0001.

FIG. 5. CD44 mediates cell aggregation via intercellular, homophilic interactions. A. Aggregation images at the 72-h time point showing that hyaluronan antagonist (o-HA) slightly increased the cluster size of L2T-labeled TN1 PDX tumor cells. Scale bars=50 μm. B. Quantitative curves of the cluster size (left panel) and number (right panel) of TN1 cells measured via IncuCyte time-lapse imaging (n=3 biological replicates, MANOVA *p<0.05). C. Images of MDA-MB-231 cell aggregation at 1 h with or without hyaluronic acid synthase inhibitor (HASi) 4-MU (at 0.4 mM/L) pre-treatment for 48 h. Scale bars=25 μm. D. Quantitative counts of aggregated MDA-MB-231 cells with cluster sizes of 2-5, 6-10 and >10 cells pretreated with or without 4-MU (n=5). NS=no significant difference. E. CD44 immunofluorescence staining with dissociated MDA-MB-231 cells during the aggregation assay 48 h post-transfection with siCon (top panel) and siCD44 (bottom panels). Most of the CD44-negative cells remained as single cells in suspension, whereas residual CD44 in knockdown cells was located at the intercellular interface of a few clusters. F. The binding curves of biotin-conjugated CD44 at 0, 1 and 5 μg/ml to the solid phase CD44 and BSA, measured as OD₄₅₀ units. T-test ***p<0.001. G. Top panel: diagram of mixed HEK-293 cell aggregates of two populations transfected with C-terminal FLAG-tagged and HA-tagged CD44, respectively. Bottom panels: immunoblots for the CD44-FLAG and CD44-HA proteins upon co-IP with anti-HA and anti-FLAG antibodies, respectively. H. Structure model of CD44s monomer (the signal peptide 1-20 not shown) with the N-terminal residues, especially Q21-C97 of the extracellular domain I and the beginning of the domain II, as predicted by computational algorithm iTasser. Warmer colors indicate higher probabilities to be at the dimer interface, as predicted by protein docking algorithm BAL. I. Two representative structure models (top and bottom panels) of predicted CD44 homodimers (formed between two neighboring cells) at an almost straight angle from protein docking. The right monomer is colored coded in the same way as in H whereas the left one is in gray for contrast (see FIG. 15 for additional structure models). J. Immunoblots for CD44-FLAG (CD44s and ΔN21-97) and CD44-HA proteins upon co-IP using anti-HA and anti-FLAG antibodies with mixed HEK-293 cell aggregates of two populations transfected with FLAG-tagged (CD44s and ΔN21-97) and HA-tagged CD44, respectively. *CD44s wildtype or mutant bands. K. Images of aggregation of HEK-293 cells for 1 h, at 48 h post transfection with CD44s-FLAG and ΔN21-97-FLAG. Scale bars=50 μm. L. Quantitative counts of aggregated HEK-293 cells, transfected with CD44s-FLAG and ΔN21-97-FLAG, in cluster sizes of 2-5, 6-10 and >10 cells. T-test ***p<0.001.

FIG. 6. CD44 promotes PAK2 pathway in tumor cell aggregates. A. The number of proteins with a >2-fold change in CD44⁺ versus CD44⁻ and siCD44 versus control comparisons: 535 out of 1377, and 382 out of 1523, respectively, with 38 proteins in common. The graph shows the canonical pathways of the 38 overlapped proteins. B. Immunoblots of PAK2 in TN1 PDX tumor cells transfected with the control siCon, siPAK2, and siCD44, at 36 h after knockdown. Loading control: β-actin. C. Relative similar mRNA levels of PAK1 and PAK2 (NS=no significant change) upon siCD44 knockdown (***p<0.001), measured via quantitative real-time PCR. D. Representative aggregate images of tdTomato⁺TN1 PDX tumor cells at 72 h aggregation upon PAK2 knockdown via siPAK2. Scale bars=50 μm. E. Quantitative analyses of cluster size (left panel) and number (right panel) of TN PDX tumor clusters upon siPAK2 knockdown, measured by IncuCyte time lapse imaging. MANOVA ***p<0.001. F. Bioluminescence images of lung colonization of the siCon, siPAK2, and siCD44-transfected TN PDX tumor cells on days 0 (D0) and 2 (D2) post-tail vein infusion (36-h post transfection). G. Quantitative bioluminescence signal curves (% of D0 signal) of reduced lung colonization of TN PDX cells upon knockdown via siPAK2 and siCD44 (n=5 mice per group). T-test *p<0.05 for both siPAK2 and siCD44 comparisons to the control siCon at both D1 and D2. H. Immunoblots for the tagged proteins PAK2-FLAG and CD44-HA upon co-IP with anti-HA (CD44) using the lysates of 293T cell aggregates, 48 h post-cotransfection with PAK2-FLAG and CD44-HA. I. IF staining images of endogenous CD44 and PAK2, and Dapi signals showing the high expression of co-localized CD44 and PAK2 at the cytoplasmic membrane of the aggregated MDA-MB-231 cells (24 h aggregation). In contrast, the single cell (white arrows) in suspension display low levels of CD44 and PAK2 which are not co-localized. Scale bar=20 μm. J. Immunoblots with MDA-MB-231 cell lysates for CD44, p-PAK2, total PAK2, p-FAK, and FAK detection at 48 h post-transfection with siCD44 and siPAK2. CD44 and PAK2 positively promote each other's protein levels and FAK phosphorylation.

FIG. 7. CD44⁺ CTC cluster association with clinical outcomes. A-C. Kaplan-Meier plot of OS (A), RFS (B), and DMFS (C) for patients with high and low CD44 (probes 31615_i or 210916_s) expression in breast tumors. Expression was dichotomized at the optimal cut point plotted in GSE3143 (n=158, left panel) and GSE7390 (n=198, right two panels). 95% confidence intervals for each group are indicated by dotted lines. Cox p=0.006, 0.026 and 0.008 as indicated. D. Kaplan-Meier plot of DMFS by PAK2 mRNA expression. High and low groups were determined using the optimal cut point in the GSE19615 human breast tumor database (n=200). Cox p=0.014. E. Kaplan-Meier plot of OS for Northwestern breast cancer patients with cluster-positive and -negative CTCs, detected by CellSearch (n=118, Log rank test p=0.0057) (Table 5). F. Representative images of a CD44⁺ cell cluster (top panels) and a single CD44⁻ CTC (bottom panels) detected in human peripheral blood via CellSearch platform staining for CD45, CD44, CK, and DAPI. Scale bar=10 μm. G. Bar graph of OS, based on the swimmer plot principle, for Northwestern breast cancer patients with CellSearch-detected CD44⁺ CTC clusters and CD44⁻ CTCs only (n=8, Log rank test p=0.0389). Two patients with CD44⁺ clusters were deceased due to disease progression (Table 6). H. Diagram of individual cell migration leading to perivascular cluster formation and intravasation. Tumor cells individually migrate to sites of intravasation on blood vessels, where CD44 mediates intercellular, homophilic protein complex formation and subsequent CD44-PAK2 interaction and FAK pathway activation. The self-interaction drives tumor cell aggregation at and within the vasculature in the primary tumor and lung metastases, due to their physical proximity. CD44 directs aggregation of detached breast tumor cells that mediate polyclonal metastasis, while single CD44− tumor cells undergo anoikis within 48 to 72 h of detachment.

FIG. 8. Vascular tumor cell cluster detection in PDXs (related to FIG. 1). A. H&E staining images of CTC clusters (left three panels) and single cells (right two panels, arrow) within the vasculature of the lung metastasis sections of various TN and E1 PDX models. Scale bars=10 μm. B. IHC image of CK-positive tumor cell clusters within a vasculature of TN PDX breast tumor section (the enlarged image of the insert see FIG. 1B). Scale bar=25 μm. C. CK IHC staining image of the vascular CTC cluster (arrow) in the TN1 PDX lung section (slide 7562). Scale bar=10 μm. D. EpCAM/CD31 IHC staining image of the vascular CTC cluster (arrow) in the TN4 PDX lung section (slide 302). Scale bar=10 μm.

FIG. 9. Polyclonal tumor cell cluster detection in TN PDXs (related to FIG. 1). A. Diagram showing the PDX mix-color implants and separate-color implants and counting of dual-color and single color lung colonies via multiphoton imaging at 6-8 weeks post implantation. Mixed-color implants include mixed eGFP+ and tdTomato+ PDX cells implanted into both left and right 4th mammary fat pads. Separate-color implants then have separate eGFP+ cells implanted into one side and the tdTomato+ cells into the other side of the 4th mammary fat pads. B. Multiphoton imaging of PDX models ex vivo at 6-8 weeks post implantation. Top left panel: image of a mixed-color PDX breast tumor of L2T (tdTomato+) and L2G (eGFP+) tumor cells at the 4th mammary fat pads. Top right panel: a dual-color CTC cluster in the peripheral blood: image merged with three fluorescence channels (Hoechst 33342+ nuclei are indicated). Scale bars=10 μm. C. Table of the list of lung colonies, dual-color and single-color, and the ratio of dual color for each mouse bearing mixed-color implants or separate-color implants (n=8 mice for each group, T test P=2.11E-06). D. Frequency of the dual-color lung colonies in mice with mixed-color implants and separate-color implants, T test *****p<0.00001. E. Left panel: diagram of separate-color implants of MDA-MB-231 cells in mice, middle and right panels: fluorescence microscope and bright field images of dual-color CTC clusters in the peripheral blood.

FIG. 10. Breast tumor cell cluster formation (related to FIG. 1). A. Intravital images of eGFP+ MDA-MB-231 tumor cell cluster formation from individually migrating tumor cells in a primary tumor. At the blood vessel surface, tumor cells (numbered 1, 2, & 3) are approached and joined by individually migrating cells 4 & 5 forming a perivascular cluster. White arrows in images at 12′ and 20′ show the cumulative paths of cells 4 and 5 respectively. Scale bar=10 μm. B. Clustered invasive tumor cells in PDX primary tumor. Top left panel: diagram of PDX tumor-bearing mouse with EGF/matrigel-containing microneedles inserted into a mammary tumor to collect invasive tumor cells in vivo. Top right: graph showing the proportion of events for single cells and multi-cell clusters. Bottom panels: images of invasive cells, both single cells and clusters formed within the microneedles (placed in a culture plate). Scale bars=20 μm.

FIG. 11. PDX-derived tumor cell aggregation (related to FIG. 2). A. Cluster-dissociated single cells capable of regenerating clusters. (1) Tumor dissociation from PDXs (td-Tomato labeled) into single cell suspension. (2) Cluster formation of dissociated tumor cells ex vivo in collagen I-coated plates (at 36 hour of aggregation). (3) Dissociate half clusters into single cells versus the other half remained in clusters. (4) To demonstrate viability and functions of paired clusters and single cells in a, both dissociated single cells and clusters were placed for aggregation assays and showing comparable clusters. Scale bars=100 μm. B. Cell death or cytotoxicity (CytotoxRed dye positivity) during 72 hour aggregation of dissociated bulk tumor cells (eGFP+) from TN PDX tumors, monitored by IncuCyte. Cells started to cluster at 3 hours and made significant clusters by 12-24 hours with minimal cell death. Majority of single cells died by 48-72 hours where clusters remained alive (white arrow pointed). Scalebars=100 μm.

FIG. 12. Tumor cell clusters arise from cellular aggregation (related to FIG. 2). A. Images of aggregated MDA-MB-231 tumor cells within 1, 24 and 96 hours (h) in suspension. Scale bars=50 μm. B. Immunoblot of Oct 3/4 and β-actinin MDA-MB-231 tumor cells in suspension during the time course of aggregation (0, 24, 48, and 96h). C. Fluorescence microscope imaging of lung colonization mediated by singles and clusters of tdTomato+PDX tumor cells at 2 and 24 hours (h) post tail vein infusion. White arrow points to the clusters formed in the lungs. Scale bars=50 μm. D. Portion of the clusters in the tumor cell event counts homed to the lungs at 2 hours (h) in C. The experiments were repeated three times (n=3) with counts of at least five images per mouse. ***Ttest p<0.001. E. IHC of human breast tumor section for CD44 and CD31. Scale bar=25 μm.

FIG. 13. CD44 in tumor cell aggregates (related to FIG. 3). A. Representative aggregation images of bulk tumor cells, sorted CD44− and CD44+ tumor cells from eGFP-labeled TN PDXs, at 4 and 72 hour aggregation. Scale bars=100 μm. B&C. Quantitative curves of clustersize (B) and number (C) of TN1 PDX cells (CD44− versus CD44+), measured over time by IncuCyte time lapse imaging shown in A (n=5 replicates, MONOVA****p<0.0001). Representative images of brightfield (left column) and CytotoxRed+cell death signal (right column fluorescent channels) for both CD44 wildtype (WT) and CRISPR/Cas9-mediated knockout (KO)MDA-MB-231 cancer cells in suspension for the aggregation assay at 0, 1 and 24 hours. D. Percentage of CytotoxRed+deadcells (%) in singles and clustered (<countable 10 cells/cluster) at 48 hours of aggregation (Ttest p<0.0001). E. CD44 splicing isoforms in PDX (TN1 and TN2). 2% agar gel image of RT-PCR products on total RNA extracts from primary tumor cells using CD44 v3 and v6 specific primers. GAPDH (G) was used as a loading control. F. Quantitative analyses of anoikis (Annexin V+ and/or propidium iodide PI+) upon CD44 knockdown (siCD44) in detached MDA-MD-231 cells in poly-Hema-treated plates within 48 hours (n=3 replicates). T-test****p<0.0001. G. Images of bright field (left column) and Cytotox Red+ cell death signal (right column fluorescent channels) for both CD44WT and CRISPR/Cas9-mediated KO MDA-MB-231 cancer cells in suspension for the aggregation assay at 0, 1 and 24 hours. Scale bar=100 μm. H. Quantitated Cytotox Red+ dead cell counts per image shown in G, 1 and 24 hours of aggregation (NS, p>0.05).

FIG. 14. CD44 OE restores cell aggregation in CD44 KO cells (related to FIGS. 3 and 4). A. Images of CD44 WT and KO MDA-MB-231 cancer cells with vector control (Vec) and CD44s overexpression in suspension for the aggregation assay at 0, 30 and 60 minutes (min). Scale bars=100 μm. B. Flow cytometer analysis of CD44 expression of both WT and KO cells upon transfection of CD44 (44) cDNA, suggesting CD44 partially restored in about 50% of MDA-MB-231 cells. C. Quantitative number of clusters shown in A. The experiments were repeated with counts of at least five images. T Test, *p<0.05, **p<0.01, ***p<0.001, ****p<0.0001. D. Flow profiles of TN1 PDX tumor cells upon CRISPR/Cas9 mediated knockout of CD44 via gRNAs targeting its exon 2. Left panel: prior to sorting; right panel: purity check of CD44 KO cells post sorting (>96.9%). E. The images of human adhesion molecule antibody array (Abcam 197434) measurement showing comparable signals for almost all molecules (undetectable E-cadherin, gray dash lines) except for the weak signal of BCAM (dash lines) in the WT cells which is absent in KO cells. The targets on the array include left column (4 replicate spots): POS1 (positive 1), ALCAM, CEACAM-1, EpCAM, ICAM-1, ICAM-3, NCAM-1, P-Cadherin, P-Selectin, VE-Cadherin; and right column (4 replicate spots): POS2 (positive 2), BCAM, E-Cadherin, E-Selectin, ICAM-2, L-Selectin, NrCAM, PECAM-1, VCAM-1, NEG (negative control).

FIG. 15. CD44 homophilic interactions required for cell aggregation and lung colonization (related to FIG. 5). A. Two alternative structure models (left and right panels) of docking-predicted CD44s homodimers (formed between two neighboring cells) where two elongated monomers form an acute angle. The right monomer is colored coded in the same way as in FIG. 5H whereas the left one is in gray for contrast. B. Immunoblots for the CD44-HA protein upon co-IP using anti-FLAG antibody with mixed 293 cell aggregates of two populations transfected with FLAG-tagged (b1: CD44s and b2: ΔN21-97) and HA-tagged CD44, respectively, in the absence or presence (b3) of anti-CD44 neutralizing antibody during 3 hour aggregation (100 μg/ml). C. Images of aggregation of MDA-MB-231 cells for up to 60 minutes (1 h), in the presence of IgG and anti-CD44 treatment. Scale bars=100 μm. D. Quantitative counts of aggregated MBA-MB-231 cells at 60 min with cluster sizes of 2-5, 6-10 and >10 cells. ***T test p<0.001. E&F. Fluorescence images of the lungs at 24 h post-tail vein infusion of TN1-PDX tumor cells treated with IgG and anti-CD44 (E), and HEK-293 cells transfected with CD44s-FLAG and ΔN21-97-FLAG (F). Scale bars=50 G Quantitative counts of aggregated clusters (>3 cells) within the lungs inhibited by anti-CD44 (TN1 PDX cells, left panel) and ΔN21-97 (HEK-293 cells, right panel). ***T test p<0.001.

FIG. 16. CD44-mediated signaling pathways (related to FIG. 6). A. The top five canonical pathways analyzed based on the list of changed proteins in A for both comparisons with two overlapped pathways: protein ubiquitination and EIF2 signaling. B. Reduced FAK protein levels of TN1 PDX-derived tumor cells within 48 hours upon CD44 knockdown. C. Immunoblot of OCT3/4 with CD44 WT and KO MDA-MB-231 cell lysates during the 72 hour aggregation. D. Aggregation images of TN1 tdTomato+ tumor cells with or without PAK inhibitor (FRAX597) at 500 nM for 72 hours. Scale bars=100 μm. E. Quantitative cluster size (top panel) and number (bottom panel) analyses of TN1 tumor cells in the presence of FRAX597 (0 and 500 nM). MONOVA **p<0.01, ***p<0.001. F. Immunoblot of PAK2 with MDA-MB-231 cells at 72 hour post control siRNA (siCon) and siPAK2 knockdowns, the smart pool (p) of all three siPAk2s and individual siPAK2-1, 2, and 3. G. Images of MDA-MB-231 cell aggregation at 3 hour upon PAK2 knockdown via pooled and individual siPAK2-1, 2, and 3. H. Non-significant changes of cell death (Annexin V+ cells) between siCon and siPAK2 (pool)-transfected MDA-MB-231 cells in suspension, measured at 24-h aggregation post transfections. I. Immunoblots for the endogenous PAK2 and CD44 upon co-IP with anti-CD44 using the lysates of MDA-MB-231 cell aggregates.

FIG. 17. Anti-EGFR blocks cell clustering. A. Clustering images (upper) and curve analyses (lower) of tumor cells from TN PDXs in the presence of IgG or anti-EGFR during clustering assays. B. Clustering images (upper) and curve analyses (lower) of tumor cells from TN PDXs treated with mab EGFR vs Cetuximab. C. Clustering images (left panel) and curve analyses (right panel) of tumor cells from TN PDXs in the presence of vehicle or EGFR inhibitor Erlotinib (1 mM or 10 mM) at 0 hour and 72 hours incubation during clustering assays.

FIG. 18. EGFR enhances CD44 mediated clustering. A. (TOP) Profile of TNM1 cells sorted based on EGFR and CD44 expression. (Bottom) Curves of the cluster number of PDX-derived TN tumor cells, sorted based on CD44 and EGFR. B. Clustering images of tumor cells, CD44+EGFR+, CD44+EGFR−, and CD44−EGFR− respectively, sorted from eGFP-labeled TN PDXs at 0 hour and 72 hours incubation during clustering assays (Right). C. Clustering images (upper) and curve analyses (lower) of tumor cells from TN PDXs in control, EGFR knockdown or CD44 knockdown during clustering assays. D. Immunoblots of EGFR, phospho-EGFR (Y845) and b-actin (loading control) of TN1-PDX derived tumor cells at the time points between 0 and 72 hours of clustering assay. E. Images of immunofluorescence staining of MDA-MB-231 cells in suspension for EGFR or pEGFR and CD44 with Dapi-stained nuclei. Curves of the cluster number of PDX-derived TN tumor cells, sorted based on CD44 and EGFR. Clustering mages of tumor cells, CD44+EGFR+, CD44+EGFR−, and CD44−EGFR− respectively, sorted from eGFP-labeled TN PDXs at 0 hour and 72 hours incubation during clustering assays (Right).

FIG. 19. miR-30c reduces cell clustering and metastasis by targeting CD44. A. Representative images of primary TN1 tumor cell clusters upon transfection with miR-30c and the scramble control (con) at 0 h, 48 h and 72 h time points. Scale bars: 100 μm. (Right) Curves of cluster numbers (upper) and cluster size (lower) of TN1 cells transfected with scramble control (con) and miR-30c, monitored by IncuCyte time lapse imaging (n=5, p<0.01 at 48 hour and p<0.001 at 72 hour). B. miR30c decreased CD44 protein levels in breast tumor cells. C. miR-30c decreased CD44 mRNA in breast tumor cells (TN1) (n=3, ****p=0.000068). D. Luciferase reporter assay of the inhibitory effects of miR-30c on the CD44 3′UTR. (n=5, ***p=0.0045). E. Bioluminescence images (BLI). F. Normalized signal change (% of day 0 signal). G. Immunoblot of EGFR upon miR30 induction and CD44 Konckdown.

FIG. 20. Inhibition of EGFR effectively blocks clustering and lung colonization. A. Timeline of treatment for FIGS. 19B&C. Tumor cells were treated overnight and then implanted through tail vein injection. B. Bioluminescence images of lung colonization of IgG or anti-EGFR treated TN1 cells on day 0, 1, and 2 post tail vein infusion. C. Histograms of bioluminescence signals of lung colonization of IgG or anti-EGFR treated TN1 cells on day 1, and 2 relative to day 0 as shown in B (n=5, ***T test P values <0.001 on day 1 and 2 between two treatments). D. Timeline of treatment for FIGS. 19E&F. Cells were co-Injected with IgG or αEGFR through tail vein injection. E. Bioluminescence images of lung colonization of IgG or anti-EGFR treated mice. F. Histograms of bioluminescence signals of lung colonization of IgG or anti-EGFR treated mice.

FIG. 21. Inhibition of EGFR blocks spontaneous metastasis. A. Timeline of treatment after palpable tumor formation in PDX model TN1. B. Bioluminescent images of tumor growth before or 1, or 17 days after treatment. C. Primary tumor signal over time, ns. D. Tumor images after 2 weeks of treatment. E. Bioluminescent images of lungs after treatment. F. Measured signal of lungs. p=0.09. G. Timeline of treatment of mice with Erlotinib or vehicle for FIGS. 19H-L. H. Bioluminescence images of orthotopically-implanted breast tumors treated with vehicle or 50 mg/kg Erlotinib (daily orally) for 4 weeks. I. BLI signal (total flux) histograms of the lungs from vehicle and Erlotinib-treated breast tumor bearing mice as shown in H. J. Bioluminescence images of dissected lungs from tumor bearing mice treated with vehicle or Erlotinib in C. K. Fluorescence images of dissected lungs from tumor bearing mice treated with vehicle or Erlotinib in C. L. Representative mix-color tumor cell clusters in blood from tumor bearing mice treated with vehicle or Erlotinib in C.

FIG. 22. EGFR activation promotes clustering and can be blocked with mabEGFRLA1. A. Clustering assay of TN2 cells treated with mabEGFR vs Cetuximab. B. Cell cluster formation of TN primary tumors in collagen I-coated plates in various culture media: Epicultcomplete (EpicultB base medium plus 5% FBS, supplements, and 0.48 g/ml hydrocortisone), EpicultBase (B base medium only), RPMI 5% FBS (RPMI with 5% FBS and 0.48 g/ml hydrocortisone); RPMI 5% FBS+EGF (with 5% FBS, 0.48 g/ml hydrocortisone, and 10 ng/ml EGF).

FIG. 23. CD44 promotes EGFR stability and activity in clusters. A. Immunoblots of phospho-EGFR (Y845), EGFR and actin of TN1-PDX tumor cells in clustering assays upon CD44 knockdown for 36 hours. B. Real-time PCR showing no significant (NS) effect of siCD44 on EGFR mRNA levels. Immunoblots of phospho-EGFR (Y845) and total EGFR of MDA-MB-231 cells in suspension for 48 hours after transfected with scramble control (con), siCD44, or siEGFR. D. Immunoblots of phospho-EGFR (Y845) in MDA-MB-231 cells, transfected with siCD44, either adherent (adh) or in suspension in the presence of proteasome inhibitor (MG-132) or endocytosis inhibitor (sucrose) for 6 hours. E. Immunoprecipitation of CD44-FLAG and detection of EGFR by immunoblot.

FIG. 24. miR30c levels in CD44 high and low breast cancer PDXs. A. Correlation of miR-30c raw counts vs CD44 raw counts in triple negative breast cancer patients; R2=−0.00646, p=0.6, n=113. B. Decreased miR-30c expression in CD44+versus CD44-tumor cells of both TN1 and TN2 models (n=3, *p=0.03 and 0.01 for TN1-1 and TN1-2 comparison respectively). C. Trend in CTC cluster reduction in mabEGFR treated mice.

FIG. 25. Clustered cells in circulation of breast cancer patients have increased expression of EGFR compared to single cells.

FIG. 26. ICAM1 is highly expressed in lung metastatic cells compared to primary tumor cells. A. A schematic showing the single cell RNA sequencing of the sorted cells from patient-derived xenografts, both primary breast tumor and lung metastasis. B. A list of upregulated genes (such as ICAM1) in the lung metastatic cells vs primary tumor cells (Loge fold change), identified by single cell RNA sequencing. C. Heatmap of the stemness signature genes in correlation with ICAM1 expression in the single cells. D. Representative IHC staining of ICAM1 expression in primary tumors, CTCs, and lung metastasis from different breast cancer patient-derived xenograft (PDX) models—M1 and M2. E. Representative ICAM1 expression in primary tumor and lung metastasis determined by flow cytometry from different breast cancer PDX models, M1, M2, M3, and CTC-092. F. Differential ICAM1 expression in CTCs of seven patients with breast cancer, detected by CellSearch. G. KM-plotter of disease-specific survival (DSS) of patients with ICAM1-high and ICAM1-low expression breast cancer (*P=0.01).

FIG. 27. ICAM1 knockdown reduces metastatic and tumorigenic ability of breast cancer cells. A-B. Representative images and quantitative data of BLI of mice infused with siCon and siICAM-1-transfected MDA-MB-231 cells via tail vein (n=4). C. Representative BLI of mice injected with siCon and siICAM-1-transfected mouse breast tumor E0771 cells via tail vein injection in immuno-competent B6 mice (n=3). D. Immunoblotting showing ICAM1 knockdown efficiency. E. Diagram of tumorigenic assay for each mouse and representative images of tumors from 4 distinct populations, orthotopically injected into 4 mammary fat pats with 100 sorted cells of ICAM1+CD44− (R2), ICAM1−CD44+ (L2), ICAM1+CD44+ (R4) and ICAM1−CD44+ (L4) MDA-MB-231 cells. F-G. BLI and tumor growth curves of the sorted cells in 5E (n=4 mice).

FIG. 28. ICAM1 mediates tumor cell clustering through homophilic interactions. A. CellSearch-analyzed representative images of CTCs (two single cells and a three-cell cluster), cytokeratin, DAPI, and ICAM1 are indicated, CD45 negative staining. B. CellSearch-based ICAM1 expression in CTC clusters versus singles. C. ICAM1 enrichment in the CTC clusters versus single CTCs in breast cancer patients (N=30 patient blood samples, 112,125 CTC events, P=0.000026), analyzed by flow cytometry (CD45−ICAM1+/− cells). D. PDX-sorted ICAM1+ and ICAM1− show different cluster formation efficiencies ex vivo. Upper panel: Representative images of cluster formation. Lower panel: Quantitative analysis of cluster formation. E. ICAM1 knockdown inhibited MDA-MB-231 cell cluster formation. Upper panel: Representative images of cluster formation assay captured by time-lapse IncuCyte live cell imaging microscopy. Lower panel: Quantitative analysis of cluster formation assay. F. Anti-ICAM1 polyclonal antibody inhibited MDA-MB-231 tumor cell clustering in vitro. Upper panel: Representative images of cluster formation. Lower panel: Quantitative analysis of cluster formation. G. ICAM-1 can form homophilic interaction. Upper panel, diagram of mixed HEK-293 cell aggregates of two population transfected with C-terminal Flag-tagged and Myc-tagged ICAM1, respectively. Lower panel, immunoblots for the ICAM1-flag and ICAM1-Myc proteins upon co-IP with anti-Myc and anti-FLAG antibodies, respectively. H. ICAM1 dimers and tetramers after treatment with the crosslinking agent DSS (left and right panels: lower and higher exposure time of film processing, respectively).

FIG. 29. ICAM1 pathways related to cancer sternness and inhibits differentiation. A. ICAM1 knockdown-altered gene pathways, analyzed by RNA sequencing B. Western blot validation of ICAM1 targets. C. ICAM1 knockdown decreased mammosphere formation ability in vitro. B. Immunoblotting showing expression of multiple stenness markers was decreased in ICAM1 knockdown cells. (3-Actin serves as loading control. D. Heatmap of the relative expression of negative regulation of cell differentiation genes (siCon vs siICAM1) for RNA sequencing data from three independent experiments. E. Flow analyses of downregulated ICAM1 and upregulated EpCAM levels upon ICAM1 knockdown. F. Immunoblotting confirmed upregulation of mammary epithelial differentiation markers in ICAM1 knockdown cells. G-J. Knockdown of ICAM1 target genes CDK6, Sec23a, and ZEB1 in decreasing mammosphere formation, tumor clustering, and migration, partially mimicking ICAM1 knockdown.

FIG. 30. ICAM1 mediates transendothelial migration of breast cancer cells. A. Diagram of transendothelial migration assay for B-C. B-C. Quantitative analysis and representative images of migrated MDA-MB-231 cells to the bottom chamber (group1: control and ICAM1 knockdown in tumor cells; group2: control and ICAM1 knockdown in endothelial cells; group3: control and ICAM1 knockdown in both tumor cells and endothelial cells.). D. Diagram of transendothelial migration assay with IgG control or anti-ICAM1 neutralizing antibody. E-F. Quantitative analysis (E) and representative images (F) of migrated MDA-MB-231 cells to the bottom chamber in the presence of absence of anti-ICAM1 neutralizing antibody. G-H. Tumor cell/endothelial cell clustering images (G) and quantitative analyses (H) upon ICAM1 knockdown in both cell types.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject” may be interchangeable with “patient” or “individual” and means an animal, which may be a human or non-human animal, in need of treatment, for example, treatment by include administering a therapeutic amount of one or more therapeutic agents that inhibit aggregation of tumor cells.

A “subject in need of treatment” may include a subject having a disease, disorder, or condition that is responsive to an inhibitor of tumor cell aggregation. For example, a “subject in need of treatment” may include a subject having a cell proliferative disease, disorder, or condition such as cancer. Cancers may include, but are not limited to adenocarcinoma, leukemia, lymphoma, melanoma, myeloma, sarcoma, and teratocarcinoma and particularly cancers of the adrenal gland, bladder, blood, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

A “subject in need of treatment” may include a subject having breast cancer. In particular, a “subject in need of treatment” may include a subject having breast cancer characterized as negative for expression of the estrogen receptor (ER), the progesterone receptor (PR), the human epidermal growth factor receptor 2 (HER2), or any combination thereof, for example, a cancer characterized as triple negative (TN) for the ER, the PR, and the HER2.

A “subject in need of treatment” may include a subject having a cancer characterized by expression of CD44, PAK2, EGFR, and ICAM1 (e.g., CD44⁺ CTCs).

A “subject in need of treatment” may include a subject exhibiting or at risk for developing circulating tumor cells (CTCs). In particular, a “subject in need of treatment” may include a subject exhibiting or at risk for developing CTCs that express CD44, PAK2, EGFR, and/or ICAM1 (e.g., CD44⁺ CTCs).

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

The disclosed therapeutic agents may be effective in inhibiting cell aggregation of tumor cells including circulating tumor cells (CTCs) and migrating tumor cells. Cell aggregation and inhibition thereof by the presently disclosed therapeutic agents may be assessed by cell aggregation methods disclosed herein and known in the art. Preferably, the disclosed therapeutic agents have an IC₅₀ of less than about 10 μM, 5 μM, 1 μM, or 0.5 μM in the selected aggregation assay. The therapeutic agents utilized in the methods disclosed herein may be formulated as pharmaceutical compositions that include: (a) a therapeutically effective amount of one or more of the therapeutic agents as disclosed herein; and (b) one or more pharmaceutically acceptable carriers, excipients, or diluents.

Therapeutic Targeting of Tumor Cell Aggregation for Treating Cancer

The disclosed subject matter relates to methods for treating cancer in a subject in need of treatment. The methods typically include administering to the subject a therapeutic agent that inhibits aggregation of tumor cells.

In some embodiments of the disclosed methods, the subject has a cancer or is at risk for developing a cancer that is characterized by circulating tumor cells (CTCs) and migrating tumor cells, and in particular CTCs and tumor cells that have properties associated with cancer stem cells (CSCs). CTCs are known in the art and are characterized as cells that have spread from established tumors and are circulating in the peripheral vasculature of a subject having the tumor, and have the capacity to form secondary tumors via metastasis. CTCs also are known in the art to account for ˜90% of solid-tumor mortality. Migrating tumor cells can be tumor cells that migrate outside the vasculature at the primary tumor site and the secondary tumor site. Both migrating tumor cells and CTCs can dynamically aggregate to increase metastatic spreading efficiencies.

In the disclosed methods, the subject typically has a cell proliferative disease or disorder such as cancer. In some embodiments of the disclosed methods, the subject has breast cancer. In particular, the subject may have a breast cancer that is characterized as being negative for one or more of the estrogen receptor (i.e., (ER)-negative breast cancer), the progesterone receptor (i.e., (PR)-negative breast cancer), the human epidermal growth factor receptor 2 (i.e., (HER2)-negative breast cancer), and/or a combination thereof (e.g., a cancer characterized as negative for all three of the ER, the PR, and the HER2 otherwise known as triple negative breast cancer (TNBC)). In some embodiments, the subject may have a breast cancer that is characterized as being positive for HER2 (i.e., HER2-positive breast cancer).

In some embodiments, the subject may have a breast cancer including circulating tumor cells (CTCs) or CTC cluster that are characterized as being negative for one or more of the estrogen receptor (i.e., (ER)-negative CTCs or CTC clusters), the progesterone receptor (i.e., (PR)-negative CTCs or CTC clusters), the human epidermal growth factor receptor 2 (i.e., (HER2)-negative CTCs or CTC clusters), and/or a combination thereof (e.g., CTCs or CTC clusters characterized as negative for all three of the ER, the PR, and the HER2 otherwise known as triple negative CTCs or CTC clusters). In some embodiments, the subject may have a breast cancer including CTCs or CTC clusters that are characterized as being positive for HER2 (i.e., HER2-positive CTCs or CTC clusters).

In the disclosed methods, the subject typically is administered a therapeutic agent that inhibits aggregation of tumor cells.

In some embodiments of the disclosed methods, the subject has a cancer or is at risk for developing a cancer that is characterized by detectable CTCs that express CD44, PAK2, EGFR, and/or ICAM1 (e.g., CD44⁺ CTCs).

CD44 is known in the art as a cell-surface transmembrane glycoprotein involved in cell-cell interactions, cell adhesion, and migration. CD44 is encoded by the CD44 presented on human chromosome 11. CD44 is known to be expressed in many mammalian cell types in the so-called “standard” isoform designated as CD44s, which comprises exons 1-5 and 16-20, as well as splicing isoform variants, so called CD44v, which comprises additional exons 6-15 as CD44v1-v10. Both CD44s and CD44v contain the N-terminal domain I (a.a. 21-97) which is required for its homophilic interactions and cellular aggregation. The self-binding regions are independent of the interaction with its known ligand hyaluronic acid.

In some embodiments of the disclosed methods, the subject is administered a therapeutic agent that is an agent that inhibits the biological activity and/or expression of CD44. In some embodiments, the therapeutic agent inhibits one or more biological activities of CD44. For example, in some embodiments, the therapeutic agent is an antibody or an antigen-binding fragment thereof that binds to CD44 and inhibits the biological activity of CD44. Antibodies that bind to CD44 are known in the art. (See, e.g., Becton Dickinson, Catalog No. 553130). In some embodiments, the therapeutic agent inhibits homophilic interactions between CD44 molecules present on the tumor cells.

In some embodiments of the disclosed methods, the subject is administered a therapeutic agent that is an agent that inhibits the biological activity and/or expression of protein activated kinase 2 (PAK2). PAK2 is one of three members of the Group I PAK family of serine/threonine kinases. PAK2 and cleaved fragments of PAK2 localize in both of the cytoplasm and nucleus. PAK2 is known in the art to modulate apoptosis, in cancers such as breast cancer. In some embodiments of the disclosed methods, the therapeutic agent inhibits the kinase activity of PAK2. Inhibitors of the kinase activity of PAK2 are known in the art and include the compound referred to as “FRAX1036” (i.e., 6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one). (See, e.g., Selleckchem, Catalog No. 7271).

In some embodiments of the disclosed methods, the subject is administered a therapeutic agent that is an agent that inhibits the biological activity and/or expression of epidermal growth factor receptor (EGFR). EGFR is a transmembrane protein that is a receptor for members of the epidermal growth factor family of extracellular protein ligands. EGFR is a member of the ErbB family of receptor tyrosine kinases. As known in the art, mutations affecting EGFR expression or activity are associated with many cancer types. In some embodiments of the disclosed methods, the therapeutic agent is an antibody or an antigen-binding fragment thereof that binds to EGFR. Antibodies against EGFR are known in the art. (See, e.g., Millipore, Catalog No. 05-101).

In some embodiments of the disclosed methods, the subject is administered a therapeutic agent that is an agent that inhibits the biological activity and/or expression of intercellular adhesion molecule 1 (ICAM1). ICAM1 also known as CD54 (Cluster of Differentiation 54) is a protein that in humans is encoded by the ICAM1 gene. ICAM1 is a cell surface glycoprotein which is typically expressed on endothelial cells and cells of the immune system. ICAM1 binds to integrins of type CD11a/CD18, or CD11b/CD18 and is also utilized by rhinovirus as a receptor for entry into respiratory epithelium. ICAM1 is a type I transmembrane protein having an amino-terminus extracellular domain, a single transmembrane domain, and a carboxy-terminus cytoplasmic domain. ICAM-1 is a ligand for LFA-1, a receptor found on leukocytes. LFA-1 has also been found in a soluble form can bind and block ICAM1. In addition, antibodies against ICAM1 are known in the art and available commercially. (See, e.g. Abcam, Anti-ICAM1 antibody [EP1442Y]—Low endotoxin, Azide free; MyBioSource.com, ICAM1 Antibody; BosterBio Anti-ICAM1 Picoband Antibody; HuaBio, Ani-ICAM1 antibody).

The disclosed methods also may include diagnostic methods. In some embodiments, the disclosed methods include methods that include detecting expression of one or more of CD44, PAK2, EGFR, and/or ICAM1 in circulating tumor cells (CTCs) of a subject having a cancer such as breast cancer. The methods further may include identifying the subject as having a high risk for developing metastatic breast cancer, for example, after having identified in the subject CD44⁺ CTCs. Optionally, the subject, thus identified, subsequently may be administered treatment, for example, treatment for cancer such as treatment for breast cancer. In some embodiments, the subject thus identified, subsequently may be administered a therapeutic agent that inhibits aggregation of tumor cells. Suitable therapeutic agents may include, but are not limited to therapeutic agents that inhibit the biological activity or expression of one or more of CD44, PAK2, EGFR, and ICAM1.

### Illustrative Embodiments

The following Embodiments are illustrative and should not be interpreted to limit the scope of the claimed subject matter.

Embodiment 1. A method for treating cancer in a subject in need of treatment, the method comprising administered to the subject a therapeutic agent that inhibits aggregation of tumor cells.

Embodiment 2. The method of embodiment 1, wherein the cancer is characterized by circulating tumor cells (CTCs).

Embodiment 3. The method of embodiment 1 or 2, wherein the cancer is characterized by CTCs that express CD44, PAK2, EGFR, or ICAM1.

Embodiment 4. The method of any of the foregoing embodiments, wherein the cancer is breast cancer.

Embodiment 5. The method of any of the foregoing embodiments, wherein the cancer is estrogen receptor (ER)-negative breast cancer, the cancer is progesterone receptor (PR)-negative breast cancer, the cancer is human epidermal growth factor receptor 2 (HER2)-negative breast cancer, and/or the cancer is triple negative breast cancer (TNBC).

Embodiment 6. The method of any of the foregoing embodiments, wherein the cancer is HER2-positive breast cancer.

Embodiment 7. The method of any of the foregoing embodiments, wherein the therapeutic agent inhibits the biological activity of CD44.

Embodiment 8. The method of any of the foregoing embodiments, wherein the therapeutic agent is an antibody or an antigen-binding fragment thereof that binds to CD44 and inhibits the biological activity of CD44.

Embodiment 9. The method of any of the foregoing embodiments, wherein the therapeutic agent inhibits homophilic interactions between CD44 molecules present on the tumor cells.

Embodiment 10. The method of any of the foregoing embodiments, wherein the therapeutic agent inhibits expression of CD44.

Embodiment 11. The method of any of the foregoing embodiments, wherein the therapeutic agent inhibits the biological activity of protein activated kinase 2 (PAK2).

Embodiment 12. The method of any of the foregoing embodiments, wherein the therapeutic agent inhibits the kinase activity of PAK2.

Embodiment 13. The method of any of the foregoing embodiments, wherein the therapeutic agents is FRAX1036 (i.e., 6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one).

Embodiment 14. The method of any of the foregoing embodiments, wherein the therapeutic target inhibits expression of PAK2.

Embodiment 15. The method of any of the foregoing embodiments, wherein the therapeutic agent inhibits the biological activity of epidermal growth factor receptor (EGFR).

Embodiment 16. The method of any of the foregoing embodiments, wherein the therapeutic agent is an antibody or an antigen-binding fragment thereof that binds to EGFR.

Embodiment 17. The method of any of the foregoing embodiments, wherein the therapeutic agent inhibits the biological activity of intercellular adhesion molecule 1 (ICAM1).

Embodiment 18. The method of any of the foregoing embodiments, wherein the therapeutic agent is an antibody or an antigen-binding fragment thereof that binds to ICAM1.

Embodiment 19. A method comprising detecting expression of one or more of CD44, PAK2, EGFR, and ICAM1 in circulating tumor cells of a subject having breast cancer.

Embodiment 20. The method of embodiment 19, further comprising identifying the subject as having a high risk for developing metastatic breast cancer.

Embodiment 21. The method of any of embodiments 19 or 20, further comprising administering to the subject a therapeutic agent that inhibits aggregation of tumor cells.

Embodiment 22. The method of any of embodiments 19-21, further comprising administering to the subject a therapeutic agent that inhibits the biological activity or expression of one or more of CD44, PAK2, EGFR, ICAM1.

### EXAMPLES

The following Examples are illustrative and should not be interpreted to limit the scope of the claimed subject matter.

### Example 1—Homophilic CD44 Interactions Mediate Tumor Cell Aggregation and Polyclonal Metastasis in Patient-Derived Breast Cancer Models

Reference is made to the manuscript authored by Liu et al., “Homophilic CD44 interactions mediate tumor cell aggregation and polyclonal metastasis in patient-derived cancer model,” to be published Oct. 25, 2018, which is incorporated herein by reference in its entirety and for which a copy is provided herewith as an Appendix to this application.

Abstract

Circulating tumor cells (CTCs) seed cancer metastases; however, the underlying cellular and molecular mechanisms remain unclear. CTC clusters were less frequently detected but more metastatic than single CTCs of triple negative breast cancer patients and representative patient-derived-xenograft (PDX) models. Using intravital multiphoton microscopic imaging, we found that clustered tumor cells in migration and circulation resulted from aggregation of individual tumor cells rather than collective migration and cohesive shedding. Aggregated tumor cells exhibited enriched expression of the breast cancer stem cell marker CD44 and promoted tumorigenesis and polyclonal metastasis. Depletion of CD44 effectively prevented tumor cell aggregation and decreased PAK2 levels. The intercellular CD44-CD44 homophilic interactions directed multicellular aggregation, requiring its N-terminal domain, and initiated CD44-PAK2 interactions for further activation of FAK signaling. Our studies highlight that CD44⁺ CTC clusters, whose presence is correlated with a poor prognosis of breast cancer patients, can serve as novel therapeutic targets of polyclonal metastasis.

Significance

CTCs not only serve as important biomarkers for liquid biopsies, but also mediate devastating metastases. CD44 homophilic interactions and subsequent CD44-PAK2 interactions mediate tumor cluster aggregation. This will lead to innovative biomarker applications to predict prognosis, facilitate development of new targeting strategies to block polyclonal metastasis, and improve clinical outcomes.

Introduction

Circulating tumor cells (CTCs) spread from established tumors, circulate within the peripheral vasculature, and lead to the development of distant metastases that account for 90% of solid tumor-related mortality. While many tumor cells may shed from a primary tumor, only an extremely small proportion of the CTCs can form secondary tumors (1-3). Both our studies and others' have shown that the clustered CTCs detectable in the peripheral blood of patients with breast cancer are associated with a worse prognosis than single CTCs (4,5). However, there is a lack of mechanistic understanding about which cellular and molecular properties enable tumor cluster formation and colonization and which targets may be employed to block this metastatic pathway.

Increasing evidence has demonstrated that cancer stem cell (CSC) properties contribute to tumor initiation, recurrence, and therapy resistance (6-17). CD44 is a well-known surface marker of CSCs in breast (9,18,19) and other tumors (20-22). However, the functional contributions of CSCs and CD44 to CTC cluster formation and polyclonal metastasis are yet to be elucidated.

We decided to dissect this mechanism of cancer metastasis by establishing and utilizing breast cancer patient-derived xenograft (PDX) models. We previously established five breast cancer PDX models (TN1-4 and E1) (17) and recently created two more orthotopic breast tumor PDXs (TN5-6), six of which (TN1-6) were triple negative (TN) for estrogen receptor, progesterone receptor, and HER2, that developed spontaneous lung micrometastases in NOD/SCID or NSG mice. Two PDXs (TN1 and TN2) showed basal-like subtype gene expression profiles based on cDNA microarray analyses (23). Human breast cancer MDA-MB-231 cells and mouse PyMT transgenic tumor models (24,25) have also been supplemented for the intravital imaging analyses as well as the cellular and molecular understanding of cluster formation.

Our studies here set out to determine how tumor cell clusters are generated in vivo, whether the CSC marker CD44 is enriched within CTC clusters and required for tumor cell cluster formation, and what downstream targets of CD44 are essential players to promote tumor cluster-mediated metastasis. Using mass spectrometry analyses, we have identified p21 protein (Cdc42/Rac)-activated kinase 2 (PAK2) as a new CD44 target. PAK2 is a member of the evolutionarily conserved group I PAK family of serine/threonine-protein kinases, along with PAK1/3 (26). The role of PAK2 in breast tumor cell cluster formation has been elucidated below.

Results

CTC cluster detection in humans and PDXs with metastatic breast cancer. CTC detection in humans is typically accomplished with blood analysis platforms such as the FDA-approved CellSearch™, which analyzes EpCAM-positive CTCs with additional cytokeratin (CK)-positive and CD45-negative markers (27). In mouse models of cancer, tumor cells are labeled by fluorescent proteins eGFP or tdTomato, and thus blood CTCs are detected in an unbiased manner using fluorescence microscopy of peripheral blood cells after depletion of erythrocytes. Complementary to blood CTC analyses, we also employed vascular CTC detection in tissue sections by histochemical staining as well as in tumor models by intravital fluorescence imaging, thereby enhancing our cellular and molecular understanding of the CTC clusters in human breast cancer.

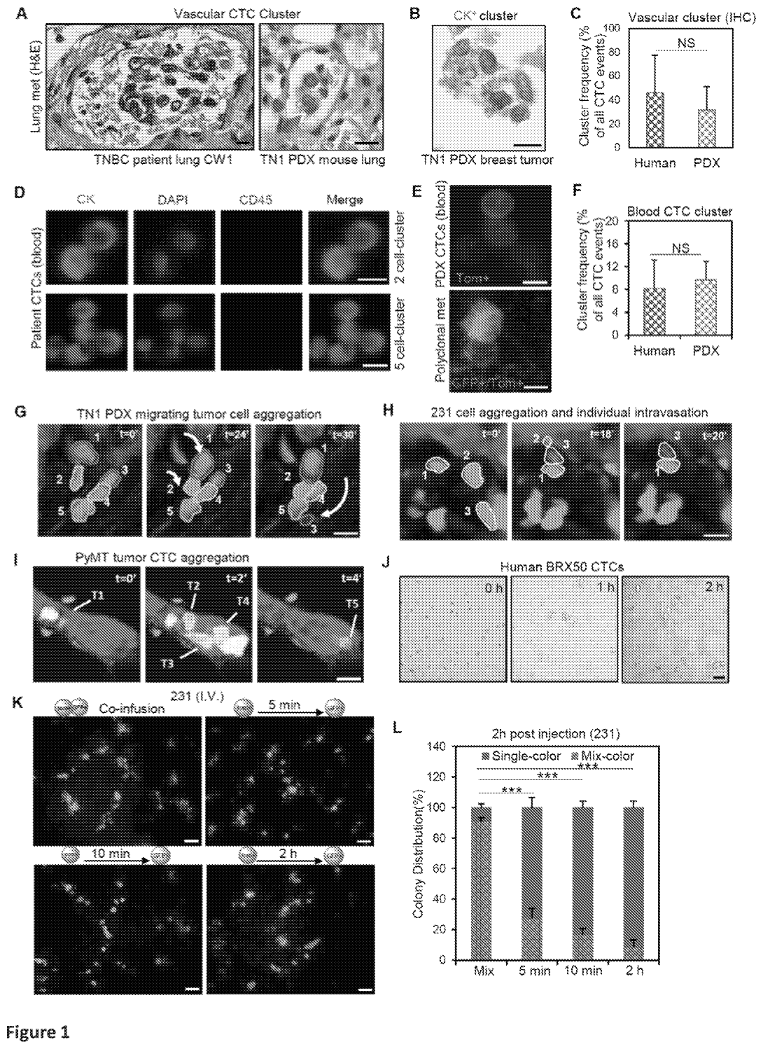

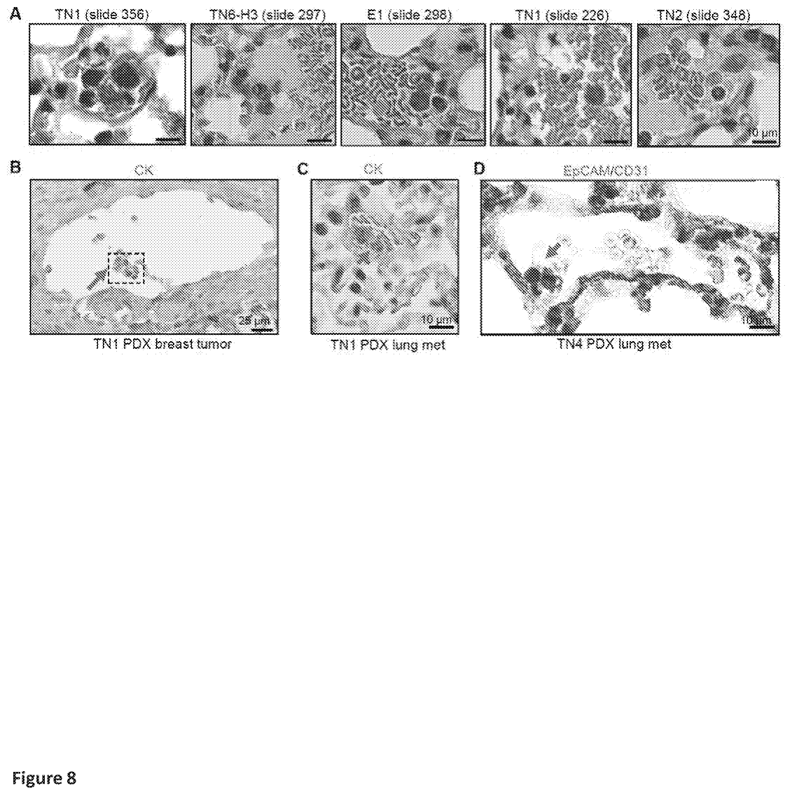

Based on tissue section availability, we first employed immunohistochemical (IHC) staining-based analyses of tissue sections, including staining with hematoxylin and eosin (H&E), epithelial markers CK or EpCAM, and endothelial marker CD31, to detect in situ CTCs within the vasculature (FIG. 1A-B, and FIG. 8A-D). We analyzed IHC-based vascular CTCs within tissue sections of primary tumors and distant metastases collected from seven breast cancer patients at Case Western Reserve University (CW1-7, n=9 tissue sections) and seven PDX models (TN1-6 and E1, n=45 lung sections from 45 mice respectively) among which TN5-6 were newly established (Table 1).

TABLE 1
Vascular CTC^{a} counts in patients and PDXs with metastatic breast cancer (HE-based or IHC-
based CK+ counts within CD31+ vasculature on one slide of tissue section per specimen)

| | | Single | Clustered | Tumor | Cluster |
|---|---|---|---|---|---|
| Patient ^{b} | IHC Slide | CTCs | CTCs | Subtype | Ratio |
| CW1 lung | Lung Mets | 88 | 33 | TN | 0.273 |
| CW1 liver | Liver Mets | 21 | 43 | TN | 0.672 |
| CW1 brain | CNS Mets | 6 | 10 | TN | 0.625 |
| CW2 breast | ‘S11-4172’ | 39 | 32 | TN | 0.451 |
| CW3 breast | ‘S10-23891’ | 2 | 10 | TN | 0.833 |
| CW4 breast | ‘S13-21915’ | 9 | 1 | TN | 0.100 |
| CW5 breast | ‘S14-19994’ | 2 | 23 | TN | 0.920 |
| CW6 breast | ‘S10-31779’ | 7 | 1 | ER−PR(2%) | 0.125 |
| | | | | Her2− | |
| CW7 breast | ‘S12-20643’ | 6 | 1 | ER−PR(1-5%) | 0.143 |
| | | | | Her2− | |
| | | | | Average | 46.02% |

| | Mouse | | IHC SLIDE | SINGLE | CLUSTERED | Tumor | Cluster |
|---|---|---|---|---|---|---|---|
| PDX | Tag | Passage# | NAME | CTCs | CTCs | Subtype | Ratio |
| TN1 lung | H2304 | P3 | (8) Slide 357 | 0 | 1 | TN | 1.000 |
| TN1 lung | E7564 | P3 | (49) Slide | 10 | 13 | TN | 0.565 |
| TN1 lung | E7562 | P3 | (50) Slide | 10 | 5 | TN | 0.333 |
| TN1 lung | E7576 | P3 | (47) Slide | 0 | 0 | TN | |
| TN1 lung | E8094 | P4 | (46) Slide | 1 | 0 | TN | |
| TN1 lung | H2054 | P7 | (28) Slide 326 | 14 | 0 | TN | |
| TN1 lung | H2229 | P8 | (16) Slide 343 | 1 | 0 | TN | |
| TN1 lung | H2227 | P8 | (17) Slide 342 | 4 | 0 | TN | |
| TN1 lung | H2225 | P8 | (18) Slide 341 | 0 | 0 | TN | |
| TN1 lung | H2219 | P8 | (19) Slide 339 | 19 | 7 | TN | 0.269 |
| TN1 lung | H2219 | P8 | (20) Slide 338 | 1 | 0 | TN | |
| TN1 lung | H2216 | P8 | (21) Slide 336 | 18 | 4 | TN | 0.182 |
| TN1 lung | H2200 | P8 | (22) Slide 335 | 2 | 2 | TN | 0.500 |
| TN1 lung | H2199 | P8 | (23) Slide 333 | 8 | 1 | TN | 0.111 |
| TN1 lung | H2189 | P8 | (24) Slide 332 | 4 | 2 | TN | 0.333 |
| TN1 lung | H2186 | P8 | (25) Slide 331 | 10 | 0 | TN | |
| TN1 lung | H2185 | P8 | (26) Slide 330 | 5 | 3 | TN | 0.375 |
| TN1 lung | H2221 | P8 | (35) Slide 306 | 13 | 13 | TN | 0.500 |
| TN1 lung | H2220 | P8 | (36) Slide 305 | 3 | 0 | TN | |
| TN1 lung | H2217 | P8 | (37) Slide 303 | 3 | 0 | TN | |
| TN1 lung | H2262 | P9 | (1) Slide 371 | 1 | 0 | TN | |
| TN1 lung | H2235/6 | P9 | (2) Slide 370 | 14 | 5 | TN | 0.263 |
| TN1 lung | H2261 | P9 | (3) slide 369 | 1 | 1 | TN | 0.500 |
| TN1 lung | H2280 | P9 | (4) slide 367 | 4 | 0 | TN | |
| TN1 lung | H2237 | P10 | (9) Slide 356 | 12 | 2 | TN | 0.143 |
| TN1 lung | H2336 | P10 | (11) Slide 352 | 2 | 0 | TN | |
| TN2 lung | H5064 | P3 | (12) Slide 350 | 4 | 0 | TN | |
| TN2 lung | H5063 | P3 | (13) Slide 348 | 8 | 1 | TN | 0.111 |
| TN2 lung | H5061 | P3 | (14) Slide 345 | 8 | 1 | TN | 0.111 |
| TN2 lung | H219.3 | P4 | (10) Slide 353 | 2 | 0 | TN | |
| TN2 lung | H2029 | P4 | (31) Slide 319 | 12 | 2 | TN | 0.143 |
| TN3 lung | H2162 | P4 | (27) Slide 328 | 4 | 1 | TN | 0.200 |
| TN4 lung | H5035 | P2 | (15) Slide 344 | 5 | 1 | TN | 0.167 |
| TN4 lung | H2009 | P2 | (38) Slide 302 | 55 | 24 | TN | 0.304 |
| TN4 lung | H2006 | P2 | (39) Slide 301 | 20 | 0 | TN | 0.000 |
| TN4 lung | H2005 | P2 | (40) Slide 300 | 16 | 5 | TN | 0.238 |
| TN4 lung | H2003 | P2 | (41) Slide 299 | 25 | 12 | TN | 0.324 |
| TN4 lung | H2003 | P2 | (42) Slide 298 | 5 | 3 | TN | 0.375 |
| TN4 lung | H2010 | P2 | (32) Slide 318 | 14 | 13 | TN | 0.481 |
| TN5-H2 lung | H310 | P1 | (33) Slide 315 | 28 | 4 | TN | 0.125 |
| TN6-H3 lung | H319 | P1 | (43) Slide 297 | 8 | 8 | TN | 0.500 |
| TN6-H3 lung | H316 | P1 | (44) Slide 296 | 9 | 2 | TN | 0.182 |
| TN6-H3 lung | H2332 | P2 | (6) Slide 363 | 4 | 2 | TN | 0.333 |
| TN6-H3 lung | H2333 | P2 | (7) Slide 360 | 1 | 0 | TN | |
| E1 lung | H253 | P2 | (45) Slide 295 | 17 | 5 | hER⁻PR⁺ | 0.227 |
| | | | | | | (lost ER in | |
| | | | | | | mice) | |
| | | | | | | Average | 31.77% |

^{a}CTCs are defined as CD45⁻CK⁺ on Cell Search or vascular tumor cells surrounded by RBCs in situ. Counts are based on one CellSearch chip or one slide of tissue section per specimen.
^{b} CW patients: Age range: 40-70 y; Race: 2 Caucasian and 4 Black, Grade: 2/3; Stage pT1-T4; ER⁻PR^{-/weak}HER2⁻; RFS: 6-14 months.
^{c}TN1-4 and E1 have been previously reported (Liu et al, PNAS 2010) and TN5-6 are newly established PDXs.

Clustered and single CTCs were detected within the vasculature of tissue sections, at similar frequencies (30-40% cluster events) and with similar morphology between human TNBC and PDX specimens (FIG. 1A-C, FIG. 8A-D and Table 1).

Using the CellSearch-based blood analysis which normally detects single CTCs, we also detected CTC clusters (FIG. 1D) in a small proportion (7 out of 46) of blood specimens collected from patients with metastatic breast cancer at Northwestern University (Table 2).

TABLE 2
Blood CTC^{a} counts in patients^{b} and PDXs with metastatic breast cancer

| | | Blood | | | | | | |
|---|---|---|---|---|---|---|---|---|
| | | draw | Total | Cluster | | | | Cluster |
| Patient # | Age | # | CTCs | # | 2-cell | 3 cells | 4 cell | Ratio |
| NU04 | 41 | 3 | 27 | 1 | 1 | | | 0.036 |
| NU06 | 52 | 2 | 135 | 7 | 4 | 2 | 1 | 0.049 |
| NU06 | 52 | 3 | 121 | 8 | 6 | 2 | | 0.062 |
| NU 10 | 29 | 1 | 609 | 9 | 9 | | | 0.015 |
| NU 017 | 47 | 2 | 23 | 3 | 2 | 1 | | 0.115 |
| NU 017 | 48 | 3 | 19 | 3 | 2 | 1 | | 0.136 |
| NU 044 | 63 | 1 | 13 | 1 | 1 | | | 0.071 |
| | | | | | | | Average | 7.64% |

| | Blood | | | | | | |
|---|---|---|---|---|---|---|---|
| | draw | Total | Cluster | | | | Cluster |
| PDX # | # | CTCs | # | 2-cell | 3 cells | 4 cell | Ratio |
| TN1 | 1 | 20 | 2 | 2 | | | 0.100 |
| TN1 | 2 | 18 | 1 | 1 | | | 0.056 |
| TN2 | 1 | 22 | 3 | 2 | 1 | | 0.136 |
| TN2 | 2 | 12 | 1 | 1 | | | 0.083 |
| TN1 | 3 | 15 | 2 | 1 | 1 | | 0.133 |
| TN3 | 1 | 17 | 1 | 1 | | | 0.059 |
| TN4 | 1 | 18 | 2 | 2 | | | 0.111 |
| | | | | | | Average | 9.66% |

^{a}CTCs are based on the CellSearch platform or L2T/L2G labeled PDX cells.
^{b}NU patients: age range 29-63 y, stage III/IV.

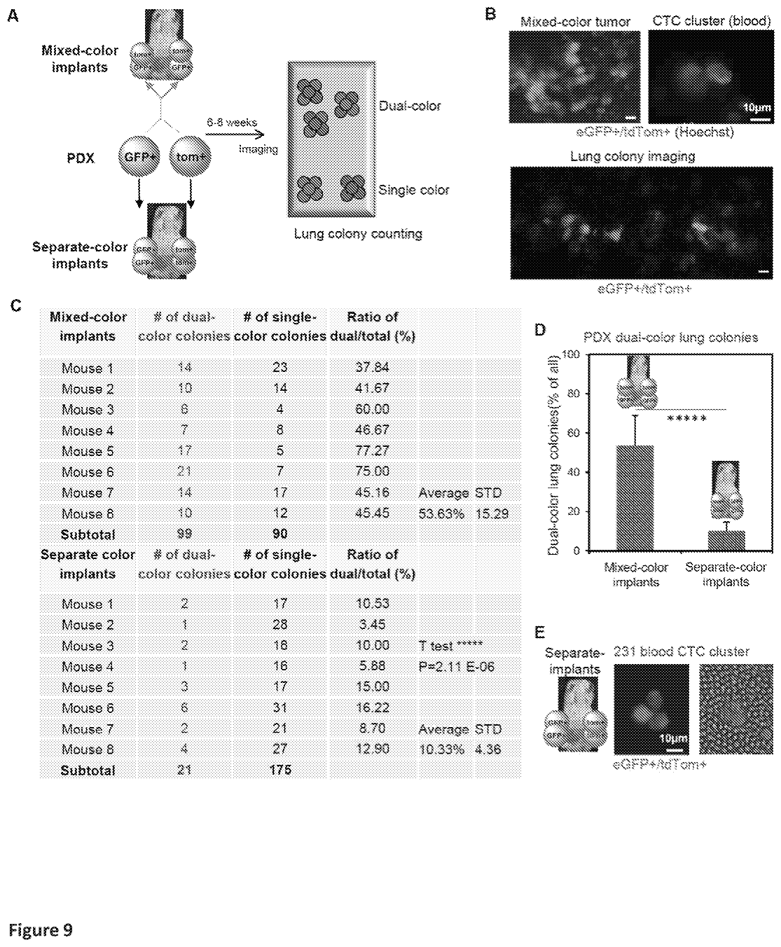

To determine whether the blood CTCs cluster in PDXs, we transduced four breast tumor PDXs with optical reporters, including eGFP, tdTomato, luciferase 2-eGFP (L2G), and luciferase 2-tdTomato (L2T), as previously described (17). In the L2G and L2T single-color and mixed-color implants of PDXs and MDA-MB-231 models, we observed both single-color and dual-color CTC clusters as well as polyclonal lung metastases of both eGFP⁺ and tdTomato⁺ tumor cells (FIG. 1E, FIG. 9A-E), which is consistent with previous reports from cell lines (5) and mouse tumor models (28). CTC clusters in the blood were detected at similar frequencies (8-10% clustered events) between patients (n=7 patients, 15 blood specimens) and PDX models (n=4 models, 7 blood specimens) (FIG. 1F, Table 2).

We then compared the frequencies of dual-color, polyclonal lung colonies between mixed-color implants and the separate-color implants of TN1 PDXs as shown in FIG. 9A. In total, a high proportion (54±15%, n=8 mice) of lung colonies of 2-10 tumor cells are dual-color in the images of mice bearing mixed L2G-L2T implants in both mammary fat pads (FIG. 9C-D), suggesting advantageous clustering from mixed tumor cells in close proximity. In contrast, a low proportion of lung colonies (10±4%, n=8 mice, p<0.0001) from separate-color implants exhibited dual-color when the L2T- and L2G-labeled tumor cells were separately implanted into two distinct fat pads on the left and the right, respectively (self-seeding or cross-seeding was not observed) (FIG. 9C-D). This suggested that these distantly separated tumor cells might meet in the vasculature or the lungs to form dual-color lung colonies.

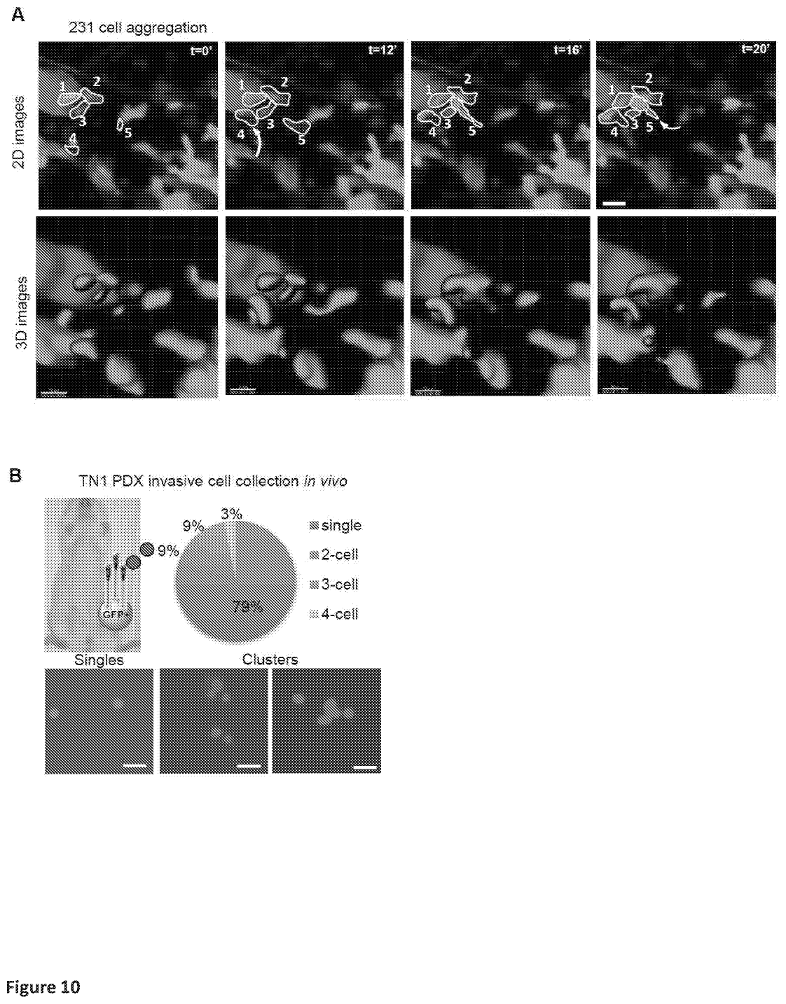

Breast tumor cell clusters arise from aggregation of individual tumor cells. In order to explore the cellular mechanisms of polyclonal cluster formation, we employed intravital multiphoton microscopic imaging of the TN1 PDX breast tumors, human MDA-MD-231 cell-derived tumor models, and mouse PyMT transgenic tumor models as described (24,25). We observed that individual migrating tumor cells aggregated into clusters near the vasculature in a dynamic touch-and-go manner (FIG. 1G, FIG. 10A, and data not shown). Some individual tumor cells detached from the cluster and subsequently entered singly into the adjacent blood vessel (FIG. 1H, and data not shown). Notably, certain CTCs remained in close proximity with each other within the tumor vasculature even during rapid blood flow, shown by snapshots and videos of intravital imaging (FIG. 1I, and data not shown).

Using the TN PDXs that display individual cell migration patterns (17,24) under intravital imaging (29,30), we collected invasive tumor cells in vivo from these models to examine the cellular patterns upon invasion into chemoattractant-containing Matrigel. We found that around 20% of all counted invasion events occurred as multicellular aggregates (FIG. 10B). We also observed that individual CTCs derived from breast cancer patient BRX-50 (31) at Massachusetts General Hospital (provided by Dr. Daniel Haber) aggregated into multicellular clusters in suspension culture within one to two hours (FIG. 1J), suggesting individual cell aggregation as a possible mechanism of CTC cluster formation.

We then sought to determine whether the frequency of polyclonal CTC aggregation and lung colonization is dependent on the timing of multiple individual tumor cells entering into blood vessels and homed to the lungs. Upon co-infusion (0 minutes apart) of both eGFP⁺ and tdTomato⁺ single breast tumor cells (MDA-MB-231 cells) via the tail vein, most of the eGFP⁺ and tdTomato⁺ tumor cells co-homed, resulting in a high ratio of dual-color aggregates of up to 5 cells (92%) within 2 hours in the lungs (FIG. 1K-L). In contrast, sequential infusion of these tumor cells with separations of 5 minutes, 10 minutes, and 2 hours led to gradually decreased ratios of dual-color aggregates (foci) in the lungs (27%, 16%, and 10%, respectively) when imaged 2 hours after the final injection (FIG. 1K-L). These studies demonstrate that given a close temporal and spatial proximity of intravasation, individual CTCs are capable of aggregating into clusters in the circulation or lung vasculature. This might contribute to the higher percentage of dual-color lung colonies in mixed-color implants than in separate-color implants as shown in FIG. 9C-D.

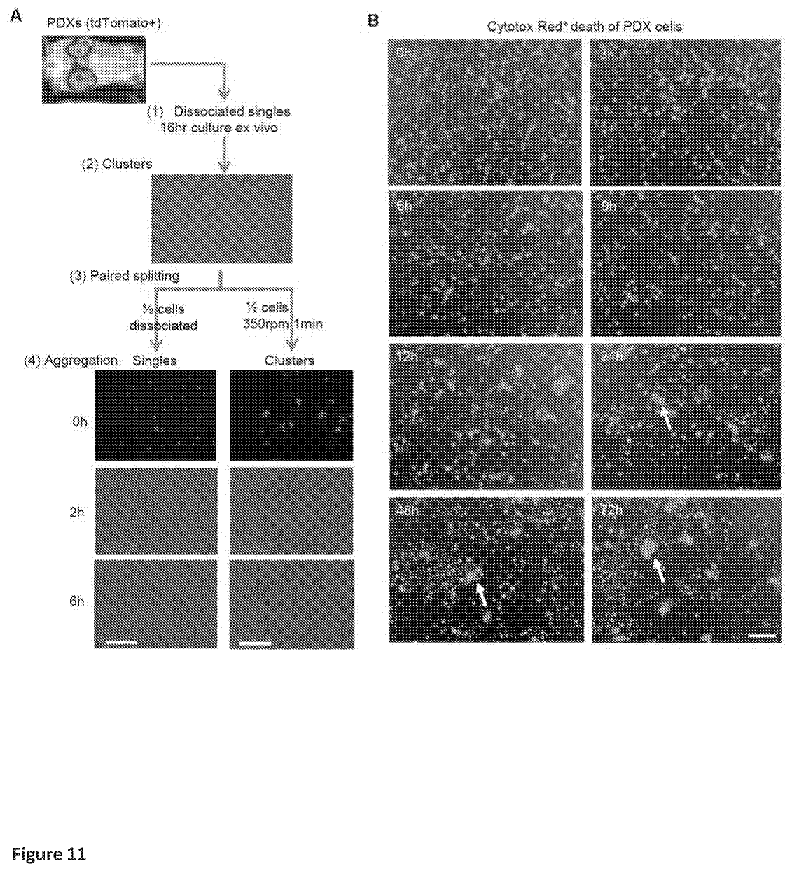

Aggregated tumor cells promote tumorigenesis and metastasis. Following this finding, we hypothesized that CSCs contribute to tumor cell aggregation and sought to determine whether tumor cell aggregates have better CSC related properties (cancer stemness) and what molecular mechanisms might underlie this aggregation phenotype. To facilitate a mechanistic understanding, we optimized an ex vivo aggregation assay with dissociated PDX tumor cells and monitored real-time cell aggregation using time-lapse IncuCyte imaging (FIG. 11A-B and data not shown). After seeding, individual primary tumor cells (tdTomato⁺ or eGFP⁺) started to aggregate within 2-3 hours and continued to form bigger clusters overnight with minimal cell divisions, monitored under the time-lapse IncuCyte microscopy imaging (automatically scanned to the same fields every two hours). Minimal cell death was observed within the first 12 hours but became more significant only in single cells after 24 hours, demonstrating the survival advantage of clustered cells (FIG. 11B).

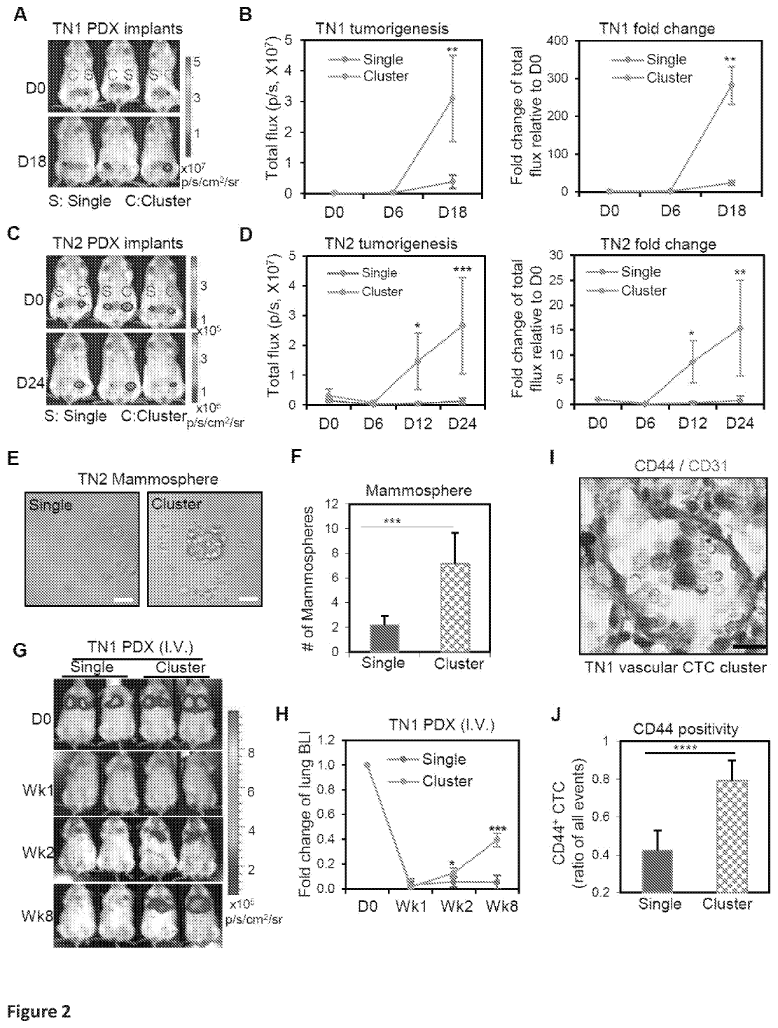

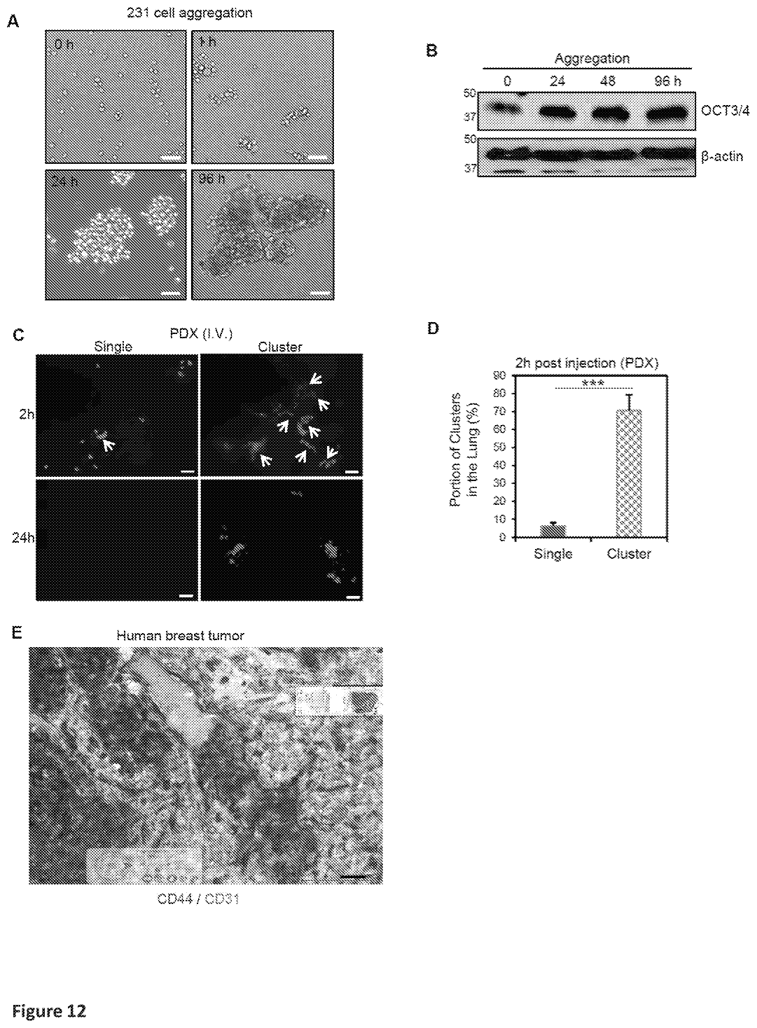

We then set out to determine the CSC-related properties (32,33) of aggregated tumor cell clusters from TN PDXs, such as orthotopic tumorigenesis (gold standard CSC assay), mammosphere formation, and lung metastasis. We orthotopically implanted single and clustered PDX cells (TN1 and TN2) in equivalent cell numbers separately into the fourth left and right mammary fat pads of each NOD/SCID or NSG mouse. Compared to the respective single tumor cells, the clusters derived from both TN1 and TN2 PDXs were more capable of initiating tumor growth, measured by bioluminescence signal intensity over 2-4 weeks (FIG. 2A-D). The clustered tumor cells from the PDXs formed mammospheres at a 3.5-fold higher efficiency compared to their single-cell counterparts in serum-free mammary epithelial stem cell media ex vivo (FIG. 2E-F). We also observed that dissociated MDA-MB-231 tumor cells formed aggregates in suspension culture as early as within one hour and continued to expand up to 96 hours, while protein levels of the pluripotency-related OCT 3/4 increased over this time frame (FIG. 12A-B).

We continued to compare the stemness-requiring metastatic potential between single and clustered tumor cells derived from TN PDXs by directly injecting tumor cells into the tail vein and examining the lungs. Unlike single MDA-MB-231 breast tumor cells, which aggregated with high efficiency within two hours of tail vein infusion (FIG. 1K), TN PDX-derived L2T⁺ single tumor cells had a low efficiency in forming aggregated clusters ≥3-5 cells (FIG. 12C-D). Meanwhile, the injected clusters had a short-term survival advantage in the lungs at 24 hours (FIG. 12C-D). While the bioluminescence signals of clustered and single tumor cells both dropped to about 2% within one week, only the aggregated tumor cells recovered and regained metastatic growth within 2-8 weeks (FIG. 2G-H), demonstrating that the clusters have CSC properties that increase their tumorigenic and metastatic potential.

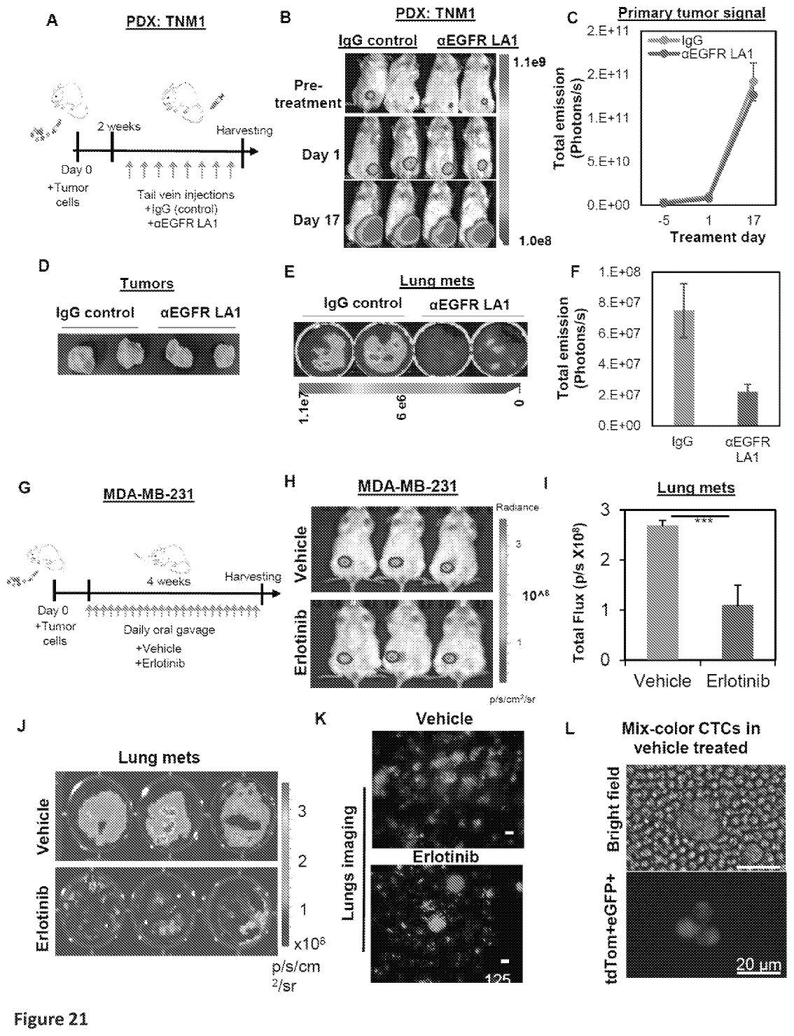

We then questioned whether markers of CSCs were detectable in clustered CTCs. CD44 and ALDH have been among the most commonly used markers of CSCs in breast and many epithelial tumors (9,18-22,34). While the ALDH signal was undetectable in TN PDXs (data not shown), we detected CD44 expression in the CTC clusters in situ within the endothelial CD31⁺ vasculatures of PDX tumor specimens and human tissues (FIG. 21, FIG. 12E). Out of 384 total counted CTC events (n=10 specimens), CD44 was notably enriched in vascular CTC clusters (100% in the PDX models, 72-90% in human sections) as opposed to single CTCs (average 42%, p=2.16×10⁻⁰⁵) (FIG. 2J, Table 3).

TABLE 3
CD44⁺ and CD44⁻ CTC Counts

| | | SINGLE CTCs | | | CLUSTERED CTCs | |
|---|---|---|---|---|---|---|
| Patient or PDX | % (44⁺) | CD44⁺ | CD44⁻ | % (44⁺) | CD44⁺ | CD44⁻ |
| Patient Lung | 32.95% | 29 | 59 | 81.82% | 27 | 6 |
| Patient Liver | 33.33% | 7 | 14 | 81.40% | 35 | 8 |
| Patient Brain | 50.00% | 3 | 3 | 90.00% | 9 | 1 |
| Patient Breast (n = 5) | 56.92% | 37 | 28 | 72.06% | 49 | 19 |
| PDX lungs (TN½) | 43.24% | 16 | 21 | 100.00% | 11 | 0 |
| Subtotal Counts | 42.40% | 92 | 125 | 79.39% | 131 | 34 |
| T Test P | 2.16E−05 | | | | | |

CD44 is required for tumor cell aggregation and lung colonization. To determine the functional importance of CD44 in tumor cell aggregation and subsequent lung colonization, we evaluated the effects of modulated CD44 levels on breast tumor cell aggregation, spontaneous lung metastasis upon orthotopic implantation, and lung colonization via tail vein injection.

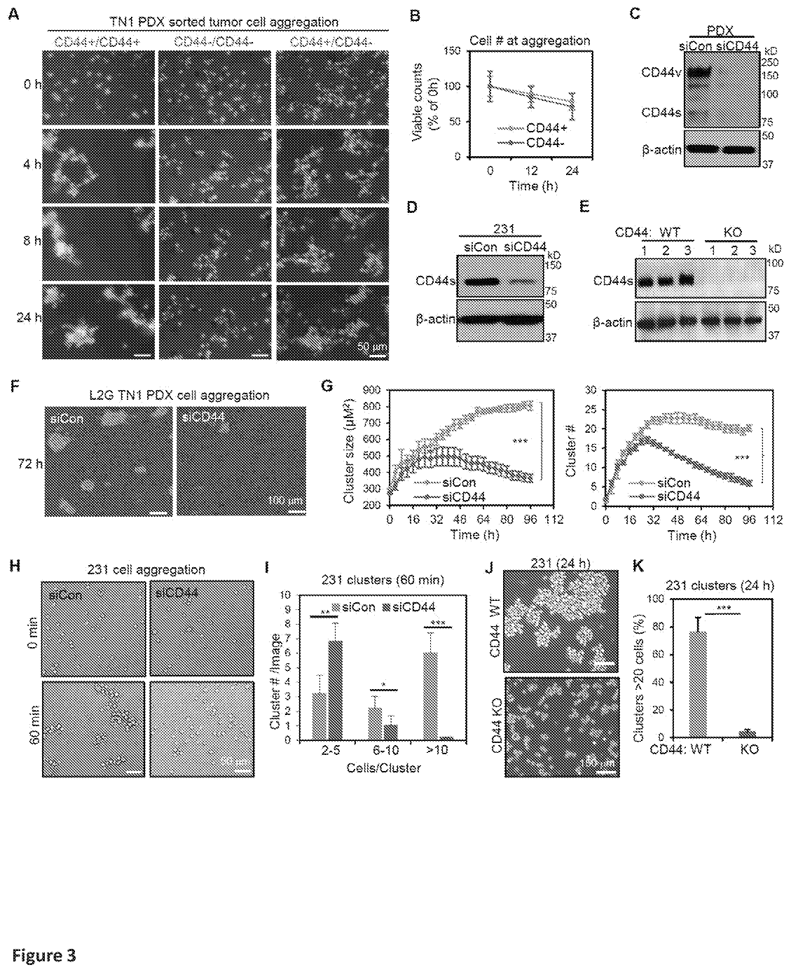

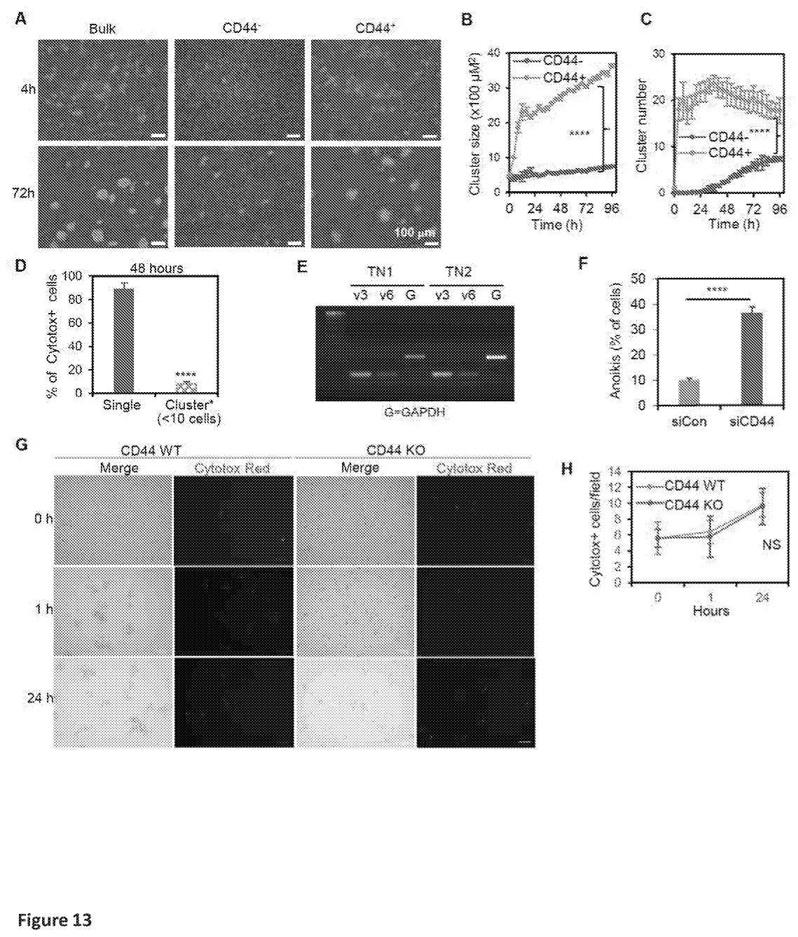

We first sorted CD44⁺ and CD44⁻ tumor cells from L2G-labeled TN PDXs for aggregation assays ex vivo and observed that CD44⁺ tumor cells formed clusters not only of a bigger size but also in a larger quantity than CD44⁻ tumor cells (FIG. 13A-C). To compare CD44⁺ and CD44⁻ cells simultaneously in a competitive cellular aggregation course, we utilized the identical TN PDXs labeled with eGFP or tdTomato (17). We first sorted CD44⁺ and CD44⁻ tumor cells separately from these PDXs, each separately color-tagged for mixed-color aggregation assays. While the double-positive mixture of eGFP⁺CD44⁺ and tdTomato⁺CD44⁺ cells formed large, dual-color aggregates together (FIG. 3A left column and data not shown), the double-negative mixture of eGFP⁺CD44⁻ and tdTomato⁺CD44⁻ tumor cells did not form comparable aggregates in either color (FIG. 3A middle column and data not shown). Furthermore, when eGFP⁺CD44⁻ tumor cells were mixed with tdTomato⁺CD44⁺ tumor cells, only the CD44⁺ tumor cells formed distinct aggregates whereas the CD44⁻ cells remained as single cells after 24 hours of ex vivo aggregation culture in the mammary epithelial stem cell medium (FIG. 3A right column and data not shown). There was no significant difference observed between the numbers of viable CD44⁺ and CD44⁻ tumor cells during the 24-hour aggregation (FIG. 3B), suggesting the effect of CD44 on cell aggregation is prior to any effects of cell survival. However, after a culture of 48-72 hours, the aggregated primary tumor cells showed a survival advantage over the non-aggregated single cells (FIG. 13D).

To examine the requirement of CD44 for tumor cell aggregation, we knocked down CD44 in L2G-labeled TN PDX tumor cells using a mixture of commercially available CD44 siRNAs. We found that PDX-derived tumor cells mainly expressed CD44 splicing variant forms (CD44v) at ˜150 kD (FIG. 3C and FIG. 13E). To determine if CD44 regulation of tumor cell aggregation is dependent on the variant isoforms, we utilized MDA-MB-231 breast tumor cells (also triple negative), which exclusively express CD44 standard form (CD44s) with a smaller molecular weight of ˜80 kD, which can be depleted by CD44 siRNA (siCD44)-mediated knockdown (FIG. 3D). To complement the transient knockdown studies, we also knocked out CD44 in eGFP (L2G) or tdTomato (L2T)-labeled MDA-MB-231 cells and TN1 PDXs using CRISPR/Cas9 technology (35) and custom-designed guide RNAs (gRNAs) targeting exon 2 of the CD44 gene (see Supplementary Methods). CD44 immunoblotting verified the depletion of CD44 in three batches of pooled knockout (KO) tumor cells (FIG. 3E).

Consistent with sorted CD44⁻ cells (FIG. 13B-C), the CD44 knockdown decreased the cluster-forming capacity of PDX-derived tumor cells, with smaller cluster size and fewer cluster numbers observed per image (FIG. 3F-G). Reduction of CD44s by siCD44 also inhibited the aggregation of MDA-MB-231 tumor cells during the first 60 minutes in suspension (FIG. 3H-I). These data suggest that the key role of CD44 in mediation of immediate tumor cell aggregation is independent of its isoforms and is separate from its suspected effect on proliferation. CD44 knockdown increased the death of detached single cells (anoikis) within 48 hours in suspension (FIG. 13F). They also support the idea that CD44 initiates cellular aggregation and subsequently prevents anoikis during the extended hours and days following detachment and circulation.

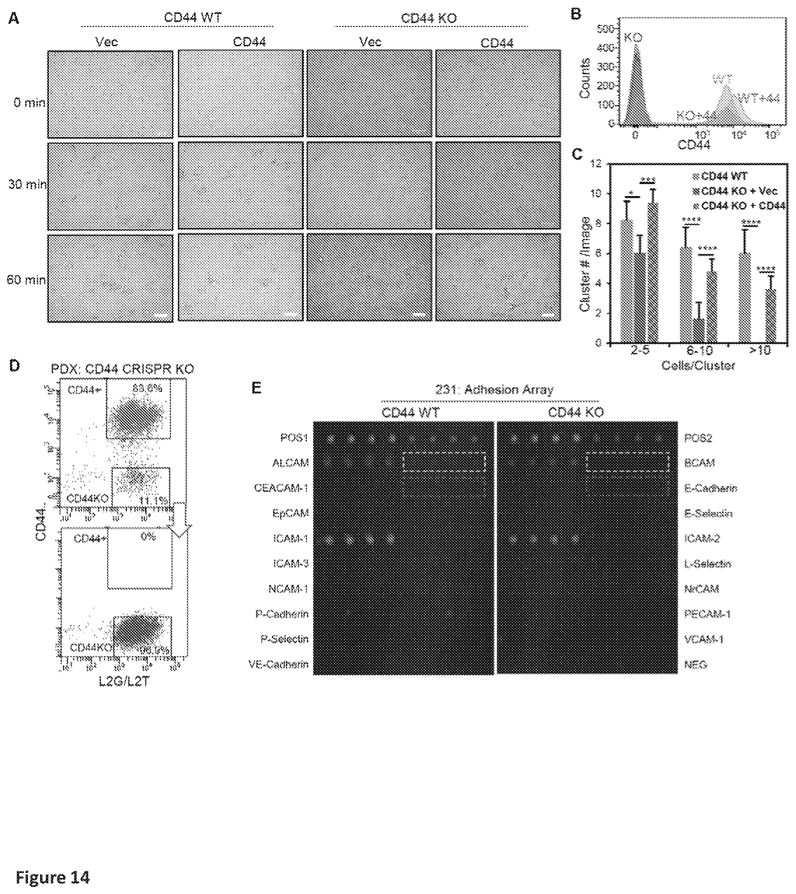

Compared to the CD44 wild-type (WT) controls, the CD44 KO cells lost aggregation capacity in vitro when measured within 24 hours (FIG. 3J-K). While the CD44 KO cells showed impaired aggregation as early as 1 hour, they did not show increased cell death compared to WT controls between 1 and 24 hours (FIG. 13G-H), further confirming that the effect of CD44 on aggregation is in parallel or prior to any potential effects on cell survival. The lost aggregation was restored by overexpression of CD44 in MDA-MB-231 KO cells (FIG. 14A-C), demonstrating that CD44 is sufficient in mediating cell aggregation.

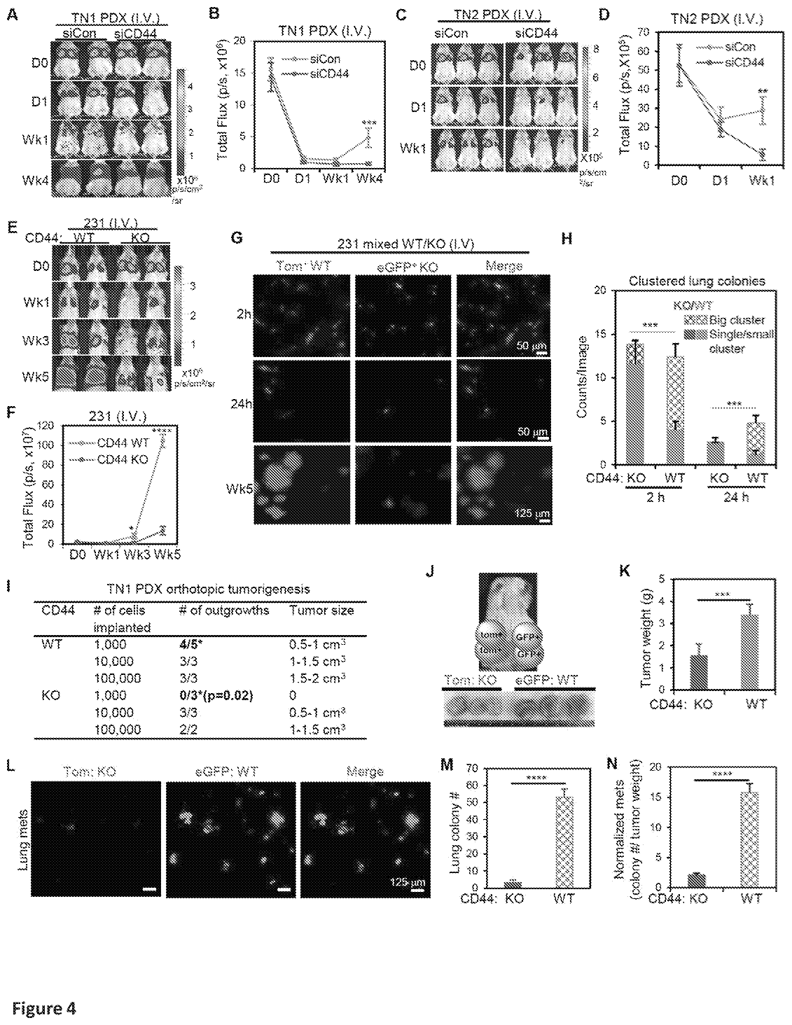

We proceeded to examine whether CD44 is required for lung colonization of aggregated CTCs in vivo. Upon tail vein infusion, the siCD44-transfected TN1 and TN2 PDX tumor cells as well as CD44 KO MDA-MB-231 cells led to a reduced efficiency of lung colonization as measured by bioluminescence imaging (FIG. 4A-F). In a competitive lung colonization assay via tail vein infusion of MDA-MB-231 cells, single eGFP⁺ CD44 KO and tdTomato⁺ CD44 WT cells (mixed at a 1:1 ratio) homed to the lungs in proximity to each other (FIG. 4G top row). A majority of tdTomato⁺ CD44 WT cells formed clusters, whereas most of the eGFP+CD44 KO cells were single cells at 2 hours post-infusion (FIG. 4G top row, 4H). By 24 hours post-infusion, few eGFP⁺ CD44 KO cells (˜5%) remained detectable, whereas ˜30% of tdTomato⁺ CD44 WT cells were visible under the fluorescence microscope, leading to a significant difference in the long-term (i.e., 5 weeks) colonization efficiency (FIG. 4G middle and bottom rows, 4H).

Using the lentiviral CRISPR/Cas9 and CD44 gRNAs, we also transduced CD44⁺ CSCs from TN1 PDXs and sorted the L2G/L2T-labeled KO cells based on surface CD44 expression and L2G/L2T (FIG. 14D). Using sorted CD44 WT and KO cells from PDXs for orthotopic implantations, we detected a reduced tumorigenic potential of CD44 KO PDX tumor cells at a low number of 1,000 cells (FIG. 4I) (p<0.05, n=5). Furthermore, on either side of the fourth mammary fat pads of each recipient mouse, we implanted a sufficient number (?100,000 cells) of tdTomato⁺ CD44 KO and eGFP⁺ CD44 WT cells (MDA-MB-231) to enable tumorigenesis into two separate tumors. These mice showed spontaneous lung metastases with a majority of CD44 WT colonies and very few CD44 KO colonies (FIG. 4J-N). While the CD44 KO tumors had a 50% reduction of tumor weight compared to the CD44 WT control tumors (FIG. 4J-K), the normalized number of lung metastatic loci per primary tumor weight of KO cells was only 1/8 that of WT cells (FIG. 4L-N). These data strongly suggest that CD44 is required for cluster formation and lung metastasis.

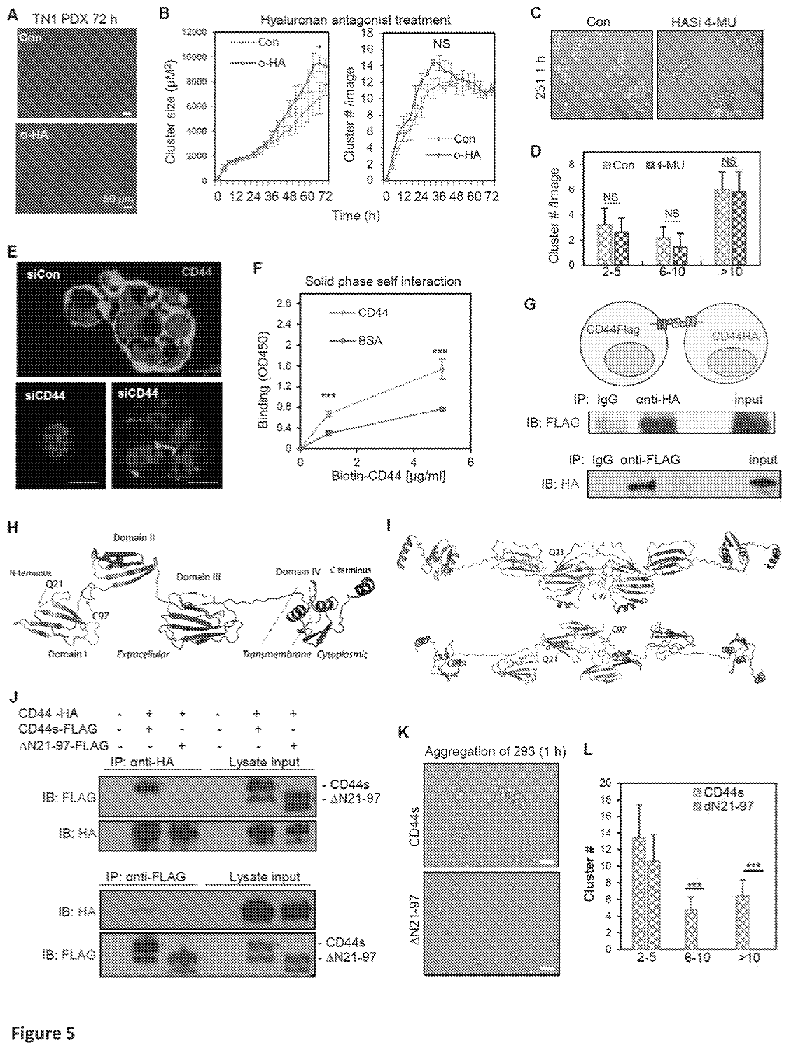

CD44 mediates intercellular, homophilic protein interactions in tumor cell aggregates. CD44 is an adhesion molecule and a known receptor for hyaluronic acid (hyaluronan) in lymphocytes (36-38). We initially speculated that hyaluronan binding to CD44 underlies the intercellular interactions of CTC aggregates. However, a hyaluronan antagonist (o-HA, provided by Dr. Bryan P. Toole) failed to block TN1 PDX tumor cell aggregation but rather slightly promoted the cluster size of tumor cell aggregates (FIG. 5A-B). Consistently, the hyaluronic acid synthase inhibitor (HASi) 4-MU did not significantly alter the aggregation of MDA-MB-231 cells in suspension (FIG. 5C-D). Therefore, CD44-directed tumor cell aggregation is hyaluronan-independent. To determine whether other adhesion molecules such as E-cadherin are involved in CD44-mediated cell aggregation, we conducted human adhesion molecule antibody array analysis for 17 major adhesion molecules with both CD44 WT and KO cell lysates. However, most of the adhesion molecules were not altered by CD44 KO, and E-cadherin was not detectable in MDA-MB-231 cells (FIG. 14E). These data suggest that CD44-mediated tumor cell aggregation is independent of its known ligand hyaluronan, E-cadherin, and other un-altered adhesion molecules.

We subsequently investigated the possibility if CD44 mediates CD44-CD44 homophilic, intercellular interactions within cell aggregates using multiple experimental approaches. Using anti-CD44 immunofluorescence staining, we first found that the residual CD44 protein was mainly located at the interface of a few tumor cell aggregates upon siCD44 knockdown (FIG. 5E). Second, we performed a solid phase self-interaction assay in vitro with His-tagged CD44 extracellular domain (ExD). CD44 ExD was immobilized to the test plates (solid phase) and displayed a significant binding to biotin-labeled CD44 ExD versus the BSA control (FIG. 5F), demonstrating a homophilic interaction between the CD44 extracellular domains. Thirdly, we overexpressed CD44 with two different C-terminal tags, CD44-FLAG (standard form CD44s) and CD44-HA (full length), into two separate sets of CD44⁻ HEK-293 cells. Upon dissociation, CD44-FLAG expressing cells were then mixed and aggregated with CD44-HA expressing cells (FIG. 5G top panel) prior to harvest and cell lysis for co-immunoprecipitation (Co-IP). Notably, the homophilic interactions between intercellular CD44-HA and CD44-FLAG proteins were reciprocally detected via the immunoblotting of CD44-FLAG protein and CD44-HA protein in the separate Co-IP pull-down lysates with the anti-HA and the anti-FLAG antibodies, respectively (FIG. 5G bottom panels). This demonstrates that intercellular CD44-CD44 interactions are responsible for mediating homophilic tumor cell aggregates.

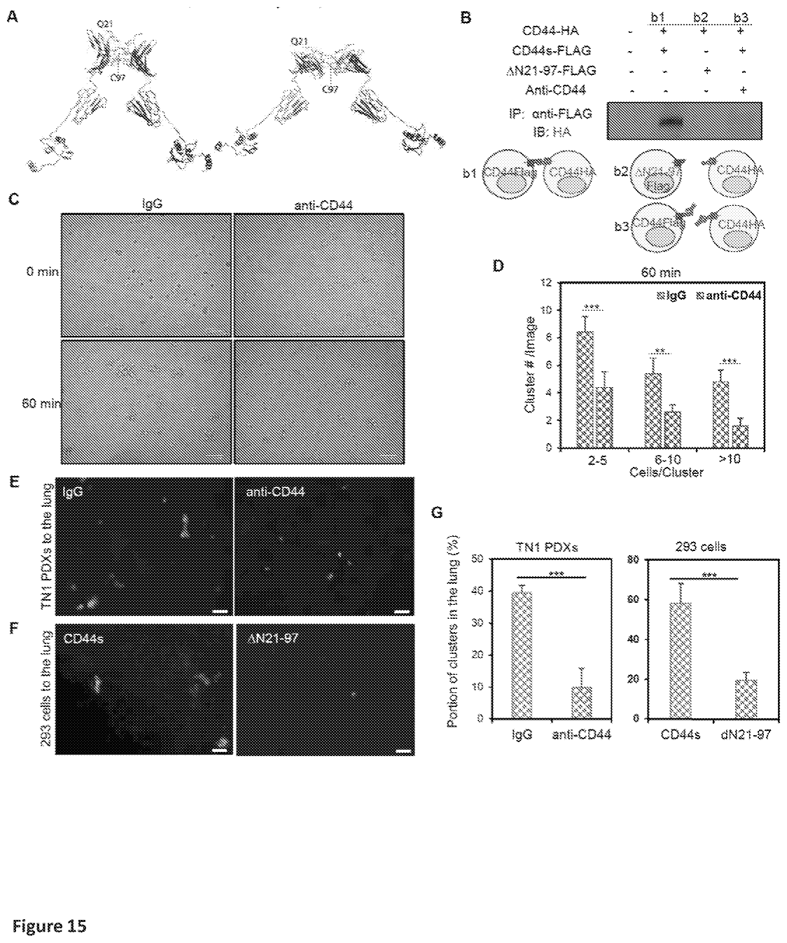

To further determine the importance of CD44 homophilic interaction in cell aggregation and lung colonization, we analyzed the CD44 sequences and structure models for subsequent studies. Based on the computational analyses and machine learning-assisted modeling, the CD44s monomer shows an elongated four-domain structure with three extracellular domains (FIG. 5H). The 10 homodimer models of CD44 from multi-stage protein docking all suggest that the N-terminal domain I (amino acids 21-97) and domain II's first a few residues are mainly responsible for the dimerization (FIG. 5I, FIG. 15A). To avoid disrupting the stability of domain structures, we therefore truncated the domain I (ΔN21-97) for subsequent Co-IP and cellular aggregation analyses. Upon transfection into HEK-293 cells, the ΔN21-97 mutant was deficient in forming intercellular complexes with CD44 (FIG. 5J) as well as losing the capacity of mediating cell aggregation (FIG. 5K-L). The N-terminal domain-dependent CD44 homophilic interaction was also blocked by an anti-CD44 neutralizing antibody when administered to aggregating cells in suspension (FIG. 15B). Meanwhile, the treatment of this anti-CD44 antibody inhibited cellular aggregation of MDA-MB-231 breast cancer cells in suspension (FIG. 15C-D). Upon tail vein injection, CD44-dependent cluster formation and colonization of TN1 PDX cells and HEK-293 cells within the lungs were further significantly diminished by the anti-CD44 blockade treatment and truncation of the CD44 self-interacting domain (ΔN21-97), respectively (FIG. 15E-G).

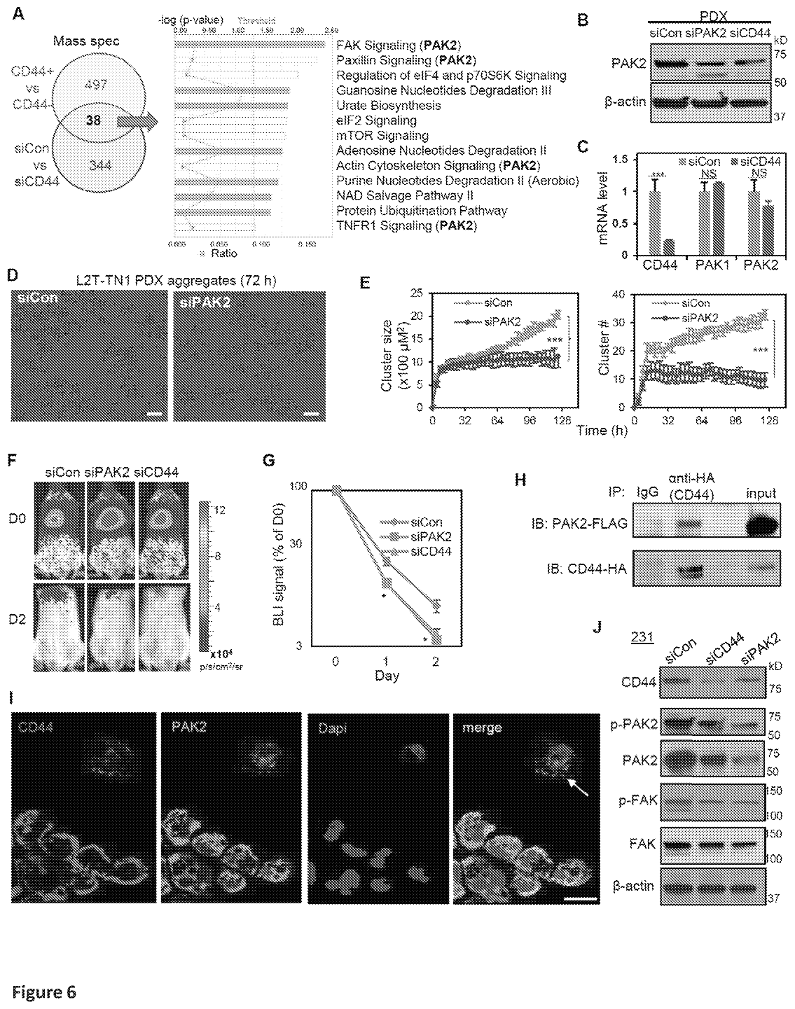

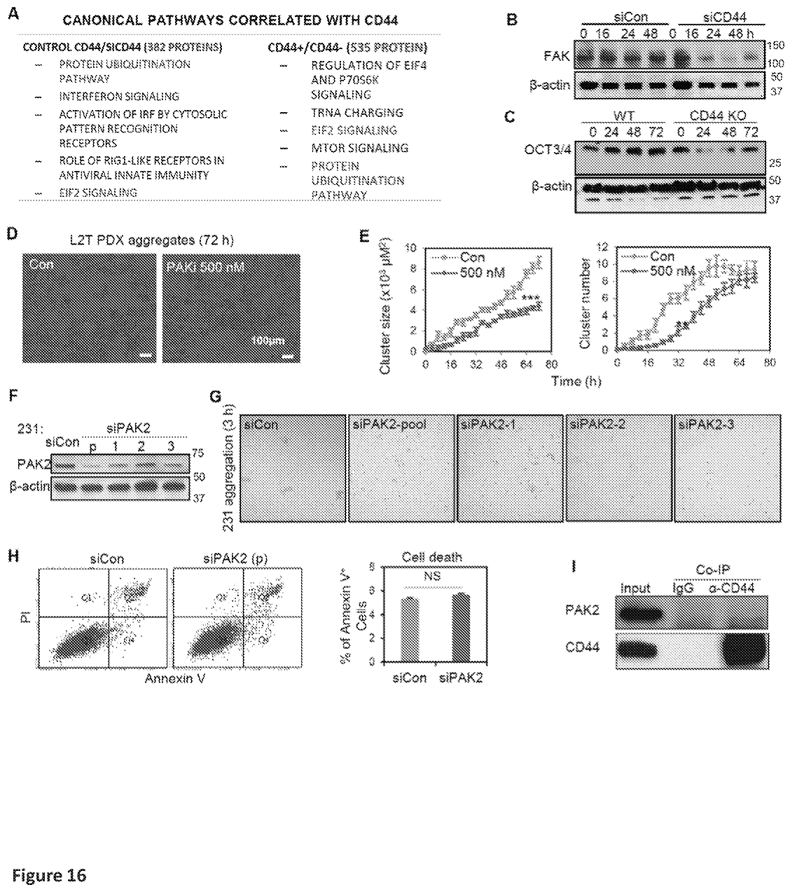

CD44 maintains PAK2 levels in tumor cell aggregates. To better understand the CD44-mediated molecular targets and downstream pathways during tumor cell aggregation, we conducted mass spectrometry analyses of sorted CD44⁺ and CD44⁻ PDX tumor cells prior to aggregation as well as the CD44 knockdown and control cells after aggregation. We identified 535 proteins and 382 proteins differentially expressed by more than 2-fold in the two comparisons (CD44+/− and siCon/siCD44), respectively (FIG. 6A, and data not shown), including two overlapping signaling pathways: protein ubiquitination and eIF2 signaling (FIG. 16A). Out of the 38 overlapping proteins regulated by CD44 in both comparisons, p21 protein (Cdc42/Rac)-activated kinase 2 (PAK2) was identified as a critical component in four of the top 13 CD44-regulated pathways such as focal adhesion kinase (FAK) signaling, paxillin signaling, actin cytoskeleton signaling, and TNFR1 signaling (FIG. 6A). PAK2 is a p21-activated kinase which activates the FAK signaling pathway as one of the three members of the evolutionarily conserved group I PAK family of serine/threonine-protein kinases, along with PAK1 and 3 (26). We further confirmed that siCD44 transfection reduced PAK2 protein levels in TN1 PDX tumor cells (FIG. 6B) along with decreased FAK protein levels during aggregation (FIG. 16B). However, the PAK2 mRNA levels were unaffected upon CD44 knockdown (FIG. 6C). While FAK had been reported to promote breast cancer stemness (39,40), the pluripotency marker OCT3/4 levels were also reduced in CD44 KO cells compared to the WT MDA-MB-231 cells (FIG. 16C), suggesting that CD44 depletion impairs cancer stemness.

To determine the importance of PAK2 in tumor cell aggregation and lung metastasis, we inhibited PAK activity using a chemical inhibitor and then knocked down its expression via siRNAs in multiple tumor cells. We found that the PAK inhibitor FRAX597 partially blocked the ex vivo aggregation of TN1 PDX tumor cells (FIG. 16D-E). We further investigated the specific effects of knocking down PAK2 on cell aggregation of PDXs and MDA-MB-231 cells. Comparable to the cluster blocking effects of siCD44, siPAK2-mediated knockdowns showed an inhibitory effect on TN1 PDX tumor cell aggregation (FIG. 6D-E). Coinciding with that, the pooled and individual PAK2 siRNAs dramatically inhibited the aggregation of MDA-MB-231 cells in suspension (FIG. 16F-G), without compromising the cell survival at the 24-hour time point of aggregation (FIG. 16H). Furthermore, the siPAK2 knockdown mimicked siCD44 in blocking the TN1 PDX tumor cell-mediated lung colonization in NSG mice (FIG. 6F-G), suggesting that PAK2 significantly contributes to CD44-mediated tumor cell aggregation and lung metastasis.

We then investigated the molecular mechanism by which CD44 regulates or sustains PAK2 protein levels. Considering that the PAK2 mRNA levels were not altered upon CD44 depletion (FIG. 6C), we hypothesized that CD44 regulates PAK2 at post-transcriptional levels, especially at protein levels. We first detected protein interactions between endogenous CD44 and PAK2 using Co-IP with the lysates of aggregated MDA-MB-231 cells (FIG. 16I). Then we confirmed the CD44-PAK2 protein complex formation using Co-IP with 293 cell aggregates expressing tagged PAK2-FLAG and CD44-HA (FIG. 6H). Lastly, based on the immunofluorescence staining of MDA-MB-231 tumor cells, endogenous CD44 and PAK2 were observed to co-localize at the plasma membrane of aggregated cells (FIG. 6I), whereas in single cells CD44 and PAK2 failed to co-localize but with a coincident reduction of both protein levels (FIG. 6I, white arrow).

To determine the downstream signaling effects of PAK2 interaction with CD44, we knocked down PAK2 in breast tumor cells and found that siPAK2 mimicked siCD44 transfection in decreasing the FAK protein levels as well as FAK activation and phosphorylation (FIG. 6J). Surprisingly, PAK2 knockdown also decreased CD44 protein levels in the tumor cells (FIG. 6J), suggesting a positive feedback and promotion between CD44 and PAK2, likely through protein complex-mediated stabilization of both proteins.

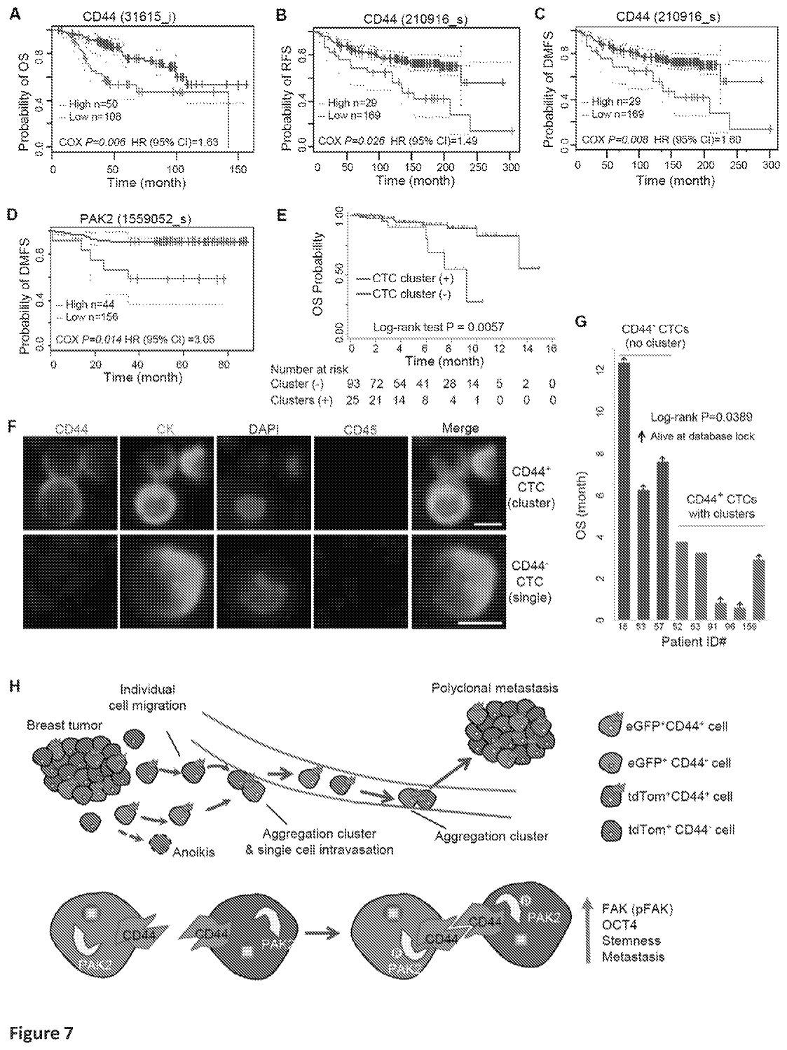

Human CD44⁺ CTC clusters associated with clinical outcomes. We next examined the clinical impact of CD44-mediated breast tumor aggregation and metastasis. Using PrognoScan (41) analyses, we observed that high levels of CD44 mRNA expression in breast tumors are associated with poor overall survival (OS), relapse-free survival (RFS), and distant metastasis-free survival (DMFS) of patients (FIG. 7A-C, Table 4).

TABLE 4
Clinical annotation of GSE diabases via PrognoScan

| | GSE3143 (CD44 | GSE7390 (CD44 | GSE7390 (CD44 | GSE19615 |
|---|---|---|---|---|
| Dataset | 31615_i) | 210916_s) | 210916_s) | (PAK2) |
| Cancer_Type | Breast cancer | Breast cancer | Breast cancer | Breast cancer |
| Subtype | | | | |
| N | 158 | 198 | 198 | 200 |
| Endpoint | Overall Survival | Relapse | Distant Metastasis | Distant Metastasis |
| | | Free Survival | Free Survival | Free Survival |
| Period | Months | Days | Days | Months |
| Cohort | Duke | Uppsala, Oxford, | Uppsala, Oxford, | DF/HCC |
| | | Stockholm, IGR, | Stockholm, IGR, | |
| | | GUYT, CRH | GUYT, CRH | |
| | | (1980-1998) | (1980-1998) | |
| Array Type | HG-U95A | HG-U133A | HG-U133A | HG-U133_Plus_2 |
| Contributor | Bild | Desmedt | Desmedt | Li |
| Data_Processing | MAS5 | MAS5 | MAS5 | dChip |
| ER | | Positive: 68% | Positive: 68% | |
| Lymph Node Status | | Positive: 0% | Positive: 0% | |
| Sample Type | Frozen | Frozen | Frozen | |
| Cutpoint | 0.68 | 0.85 | 0.85 | 0.9 |
| Minimum P-Value | 0.000246 | 0.000734 | 0.001602 | 0.000564 |
| Corrected P-Value | 0.007690 | 0.019642 | 0.037726 | 0.031699 |
| In(HR_{high}/HR_{low}) | 1.01 | 0.83 | 0.89 | 1.71 |
| COX P-Value | 0.006336 | 0.025639 | 0.008221 | 0.013876 |
| In(HR) | 0.49 | 0.4 | 0.47 | 1.12 |
| HR [95% CI] | 1.63 [1.15-2.31] | 1.49 [1.05-2.11] | 1.60 [1.13-2.26] | 3.05 [1.25-7.43] |

Consistently, high levels of PAK2 mRNA expression coincided with poor DMFS of breast cancer patients (FIG. 7D, Table 4). Taking advantage of the CellSearch platform-based blood analysis in patients with breast cancer, we confirmed the worse OS of patients with detectable CTC clusters versus the patients with single CTCs only (FIG. 7E, Table 5, n=118 patients with metastatic breast cancer).

TABLE 5
CTC test on CellSearch platform in MBC for protocol NU16B06

| | | | | Time to |
|---|---|---|---|---|
| | Total | Clusters | Clusters | cluster |
| | CTCs at | number at | detected | detection |
| ID | baseline | baseline | longitudinally | (months) |
| 1 | 8 | 0 | no | |
| 2 | 0 | 0 | no | |
| 3 | 4 | 0 | no | |
| 4 | 14 | 0 | yes | 4.93 |
| 5 | 0 | 0 | no | |
| 6 | 158 | 7 | yes | 0.00 |
| 7 | 26 | 0 | no | |
| 8 | 0 | 0 | no | |
| 9 | 28 | 0 | no | |
| 10 | 609 | 9 | yes | 0.00 |
| 11 | 2 | 0 | yes | 3.75 |
| 12 | 33 | 0 | no | |
| 13 | 0 | 0 | no | |
| 14 | 0 | 0 | no | |
| 15 | 0 | 0 | no | |
| 16 | 240 | 0 | no | |
| 17 | 31 | 0 | yes | 2.10 |
| 18 | 1 | 0 | no | |
| 19 | 72 | 0 | no | |
| 20 | 0 | 0 | no | |
| 21 | 0 | 0 | no | |
| 22 | 3 | 0 | no | |
| 23 | 0 | 0 | no | |
| 24 | 0 | 0 | no | |
| 25 | 12 | 1 | yes | 0.00 |
| 26 | 0 | 0 | no | |
| 27 | 11 | 0 | yes | 4.83 |
| 28 | 0 | 0 | no | |
| 29 | 0 | 0 | no | |
| 30 | 0 | 0 | no | |
| 31 | 0 | 0 | no | |
| 32 | 5 | 0 | yes | 11.93 |
| 33 | 0 | 0 | no | |
| 34 | 37 | 0 | yes | 3.88 |
| 35 | 0 | 0 | no | |
| 36 | 0 | 0 | no | |
| 37 | 0 | 0 | no | |
| 38 | 41 | 3 | yes | 0.00 |
| 39 | 0 | 0 | no | |
| 40 | 4 | 0 | no | |
| 41 | 1 | 0 | no | |
| 42 | 4 | 0 | no | |
| 43 | 2 | 0 | no | |
| 44 | 13 | 1 | yes | 0.00 |
| 45 | 0 | 0 | no | |
| 46 | 1 | 0 | no | |
| 47 | 72 | 0 | no | |
| 48 | 0 | 0 | no | |
| 49 | 0 | 0 | no | |
| 50 | 1 | 0 | no | |
| 51 | 0 | 0 | no | |
| 52 | 2 | 0 | no | |
| 53 | 24 | 0 | no | |
| 54 | 5 | 1 | yes | 0.00 |
| 55 | 0 | 0 | no | |
| 56 | 0 | 0 | no | |
| 57 | 3 | 0 | no | |
| 58 | 0 | 0 | no | |
| 59 | 0 | 0 | no | |
| 60 | 3 | 0 | yes | 5.19 |
| 61 | 0 | 0 | no | |
| 62 | 0 | 0 | no | |
| 63 | 904 | 140 | yes | 0.00 |
| 64 | 70 | 0 | yes | 4.67 |
| 65 | 7 | 0 | no | |
| 66 | 5 | 0 | no | |
| 67 | 30 | 0 | yes | 1.81 |
| 68 | 10 | 0 | no | |
| 69 | 0 | 0 | no | |
| 70 | 120 | 14 | yes | 0.00 |
| 71 | 0 | 0 | no | |
| 72 | 0 | 0 | no | |
| 73 | 0 | 0 | no | |
| 74 | 0 | 0 | no | |
| 75 | 0 | 0 | no | |
| 76 | 0 | 0 | no | |
| 77 | 0 | 0 | no | |
| 78 | 0 | 0 | no | |
| 79 | 0 | 0 | no | |
| 80 | 2 | 0 | no | |
| 81 | 0 | 0 | no | |
| 82 | 17 | 5 | yes | 0.00 |
| 83 | 4 | 0 | no | |
| 84 | 15 | 0 | no | |
| 85 | 5 | 0 | no | |
| 86 | 11 | 0 | no | |
| 87 | 0 | 0 | no | |
| 88 | 1 | 0 | no | |
| 89 | 0 | 0 | no | |
| 90 | 0 | 0 | no | |
| 91 | 2 | 0 | yes | 3.16 |
| 92 | 409 | 9 | yes | 0.00 |
| 93 | 328 | 8 | yes | 0.00 |
| 94 | 5 | 0 | no | |
| 95 | 45 | 2 | yes | 0.00 |
| 96 | 10 | 0 | no | |
| 97 | 1000 | 0 | no | |
| 98 | 9 | 0 | no | |
| 99 | 12 | 0 | no | |
| 100 | 0 | 0 | no | |
| 101 | 22 | 0 | no | |
| 102 | 634 | 6 | yes | 0.00 |
| 103 | 71 | 0 | yes | 1.61 |
| 104 | 1 | 0 | no | |
| 105 | 1 | 0 | no | |
| 106 | 0 | 0 | no | |
| 108 | 137 | 11 | yes | 0.00 |
| 109 | 0 | 0 | no | |
| 110 | 2 | 0 | no | |
| 111 | 0 | 0 | no | |
| 112 | 0 | 0 | no | |
| 113 | 39 | 2 | yes | 0.00 |
| 114 | 251 | 14 | yes | 0.00 |
| 115 | 0 | 0 | no | |
| 116 | 2 | 0 | no | |
| 117 | 0 | 0 | no | |
| 118 | 28 | 0 | no | |

We also found that CD44⁺ CTC clusters in human blood were associated with lower OS than CD44⁻ CTCs (FIG. 7F-G, Table 6).

TABLE 6
Blood CTC CD44 status and clinical information of Northwestern
patient cohort, measured by CellSearch.

| | | | | OS | Survival | CD44+ | | CD44+ | |
|---|---|---|---|---|---|---|---|---|---|
| NU ID | Age | Sex | Treatment information | (months) | status | singles (%) | | clusters (%) | |
| 018 | 64 | F | lapatanib/capecitabine/ | 11.38 | alive | 0 | | 0 | |
| | | | trastuzumab | | | | | | |
| 052 | 68 | F | docetaxel/pertuzumab/ | 3.45 | deceased | 2 | (0) | 3 | (100%) |
| | | | trastuzumab | | | | | | |
| 053 | 40 | F | everolimus/trastuzumab/ | 5.75 | alive | 24 | (0) | 0 | |
| | | | pertuzumab/navelbine | | | | | | |
| 057 | 46 | F | carboplatin/everolimus | 7.00 | alive | 3 | (0) | 0 | |
| 063 | 69 | F | ixabepilone/trastuzumab/ | 2.96 | deceased | 924 | (16.1%) | 164 | (45%) |
| | | | pertuzumab | | | | | | |
| 091 | NA | F | abraxane/trastuzumab/ | 0.81 | alive | 13 | (21.4%) | 1 | (100%) |
| | | | lapatinib | | | | | | |
| 096 | NA | F | ipilumumab/nivolumab | 0.56 | alive | 60 | (19%) | 255 | (81%) |
| 159 | NA | F | capecitabine | 2.68 | alive | 400 | (4%) | 4 | (100%) |

Overall, these results identify cellular aggregation of individually migrating and circulating tumor cells as a new mechanism of tumor cell cluster formation in breast cancer, which is directly mediated by intercellular CD44-CD44 homophilic interactions and dependent on CD44-PAK2 complex-activated downstream pathways (such as FAK and OCT3/4) to promote cancer stemness and metastasis (FIG. 7H).

Discussion

Taken together, our studies demonstrate a novel mechanism of human tumor cluster formation via CD44/PAK2-mediated cellular aggregation using representative PDX models and cell lines in combination with clinical studies.

Within the past decade, CTC analyses have become an important real-time approach for cancer diagnostic and prognostic studies. Multiple technologies have been developed for CTC detection and analysis (e.g., microchip-based capture) and have greatly advanced our understanding of the polyclonal biology of tumor metastasis (42-45). Polyclonal tumor cell clusters have also been detected in additional solid tumors such as pancreatic cancer (46). Our study has unveiled the dynamics of cellular migration and aggregation leading to tumor cell cluster formation prior to and after intravasation. We propose that this new mechanism may act in addition to the previously proposed model of collective migration and cohesive shedding of polyclonal CTC clusters (5,28,47) and that there may be a possible interplay or synergy between the two mechanisms in cluster formation and transportation. The retention of CTC clusters in the capillaries of distant organs may be capable of stopping blood flow and generating a new microenvironmental niche for metastatic tumor regeneration. It is also possible for CTC clusters to reversibly break down into individual cells prior to extravasation, similar to the process of individual cell departure from clusters with subsequent intravasation. An in vitro study using microfluidic devices designed to mimic human capillary constrictions indicated that CTC clusters have the ability to dynamically break down and reform cell-cell junctions and traverse capillary-sized vessels (48).

Our studies suggest a new mechanism of homophilic interactions by which the CSC marker CD44 directs CTC aggregation to promote polyclonal metastasis. CD44 is known to bind to its ligand hyaluronan in lymphocytes; however, CD44-mediated tumor cell aggregation is independent of the hyaluronan-ligand binding, but is mediated by its intercellular, homophilic interactions, similar to other adhesion molecules such as E-cadherin (49) and PECAM1 (50). Notably, the N-terminal domain responsible for CD44 homophilic interactions also harbors most of the known hyaluronan-binding sites (51). While E-cadherin and other tight junction components mediate collective migration of tumor cell clusters (5,28), CD44-mediated cell aggregation to form tumor cell clusters is E-cadherin-independent and occurs through a distinct pathway that interacts with and activates PAK2 kinase and subsequently FAK signaling. While FAK is known to play an important role in cancer stem cells and cancer progression (39,40), PAK2 is relatively less studied. Limited studies have reported that mouse Pak2 KO results in embryonic lethality with impaired somite development and growth retardation (52). Murine Pak2 and its kinase activity are required for homing of hematopoietic stem and progenitor cells to the bone marrow (53). Human PAK2 regulates apoptosis (54) and drives tamoxifen resistance in breast cancer (55). All of these implicate a pivotal role of PAK2 in normal stem cell functions and cancer progression. Our studies further demonstrate the role of PAK2 in tumor cell aggregation and CSC-mediated metastasis.

Our work indicates that the high efficiency of CTC clusters in mediating metastasis is due not only to their advantageous survival, but also to their CSC properties and CD44-mediated signaling pathways. We propose that the increased sternness of clusters enables their plasticity and regenerative potential (32,33), leading to enhanced adaptation to new microenvironments and improved secondary tumor growth. While CTCs provide advantages for non-invasive dynamic monitoring of cancer progression, the understanding of the sternness and molecular mechanisms of CTC clusters will also improve both diagnostic prediction and development of therapeutics that prevent and block polyclonal metastasis. Regardless, our results show that CD44-mediated cell aggregation can, in parallel or subsequently, promote cell survival which is certainly required for CSC functions such as self-renewal and metastasis. Future studies may address how CTC clusters crosstalk with other blood cells such as macrophages (56) and platelets (57) in metastasis.

Methods

Human specimen analyses. All human blood and tumor specimen analyses complied with NIH guidelines for human subject studies and were approved by the Institutional Review Boards at Northwestern University and Case Western Reserve University/University Hospitals. The investigators obtained informed written consent from all subjects whose blood specimens were analyzed. Consent was waived for the IHC staining of archived tumor specimens.

Animal studies. All mice used in this study were kept in specific pathogen-free facilities in the Animal Resources Center at Northwestern University, Case Western Reserve University and Albert Einstein College of Medicine. All animal procedures complied with the NIH Guidelines for the Care and Use of Laboratory Animals and were approved by the respective Institutional Animal Care and Use Committees. Animals were randomized by age and weight. The exclusion criterion of mice from experiments was sickness or conditions unrelated to tumors. Sample sizes were determined based on the results of preliminary experiments, and no statistical method was used to predetermine sample size. All of the patient-derived xenograft (PDX) tumors were established and orthotopic tumor implantation was performed as described previously (9,17).

Cell lines and transfections. MDA-MB-231 and HEK-293 cells were purchased commercially from ATCC, and periodically verified to be mycoplasma-negative using Lonza's MycoAlert Mycoplasma Detection Kit (Cat #LT07-218). Cell morphology, growth characteristics, and microarray gene expression analyses were compared to published information to ensure their authenticity. Early passage of cells (<20 passages) were maintained in DMEM with 10% FBS+1% penicillin-streptomycin (P/S). Primary tumor cells were cultured in HuMEC-ready medium (Life Technologies) plus 5% FBS and 0.5% P/S in collagen type I (BD Biosciences) coated plates. miRNAs (Dharmacon, negative control #4) and siRNAs (pooled) (Dharmacon, negative control A) were transfected using Dharmafect (Dharmacon) at 100 nM, and re-transfected on the following day. For overexpression experiments in HEK-293 cells, pCMV6-Flag-CD44 (OriGene), pCMV3-HA-CD44, pCMV3-Flag-PAK2 and pCMV3-HA-PAK2 (Sino Biological) plasmids were transfected into cells by PolyJet (SignaGen Laboratories). After 48 hours, cells were collected for Co-immunoprecipitation and western blotting.

CD44 Structure Modeling. A 3-dimensional structure model of CD44 antigen isoform 4 precursor (CD44s) (https://www.ncbi.nlm.nih.gov/protein/48255941) was first built using the webserver iTasser (58). Two copies of CD44s monomer models were rigidly docked into each other using the webserver ClusPro (59) under the homodimer mode. The top 10 distinct homodimer models were then subject to flexible refinement using a Bayesian active learning (BAL) method where the direction and the extent of backbone conformational flexibility is sampled with protein complex-based normal mode analysis cNMA (60,61). The 10 refined models were re-ranked with BAL-determined probabilities as weights. Moreover, residues were also assigned probabilities based on the weighted models. Specifically, each residue in each model was assigned the model's probability if it is at the model's putative interface (defined by a 5-A distance cutoff between homodimeric heavy atom pairs) and a zero otherwise; and each residue had these values across all 10 models summed into the residue's probability ranging from 0 to 1. For instance, if a residue appears at the putative interface of all 10 distinct models, its probability score will be 1. Due to the symmetry of the homodimer, each residue's probability is further averaged over both chains in this study. Structure models were visualized using the molecular graphics program PyMol (62). The predicted “hotspot” residues are concentrated over the first 97 residues where C97 at the first inter-domain linker is suggested to form disulfide bonds across some predicted dimer interfaces. As protein docking was performed without the environment of the membrane, the first two homodimer models in FIG. 5I feature an almost straight angle between the two monomers, which would need drastic inter-domain conformational changes to accommodate middle domains in a membrane. The alternate two models in FIG. 15A forming an acute such angle, would not have to do so and are potentially more likely.

Statistical analysis. For all assays and analyses in vitro, if not specified, a two-tailed Student's t-test performed using Microsoft Excel was used to evaluate the p-values, and p<0.05 was considered statistically significant. Data are presented as mean±standard deviation (SD). For all the IncuCyte clustering assays, biological triplicates were performed. For all other cell-based in vitro experiments, three technical replicates were analyzed. For animal studies in vivo, cluster curves were analyzed using Wilcoxon rank sum tests and MANOVA analyses in R software. For the fluorescence lung imaging, at least 5 random fields of the lung from each mouse were taken, and at least 3 mice were used. Spearman-Brown reliability coefficients were calculated for varying number of repeats in order to find the number of technical replicates required to attain a reliability of 90% (63,64). For animal studies, we determined the group size using Bonferroni correction for multiplicity, we set α=0.05/4=0.0125. We assumed the mean difference between the groups was at least twice as much as the standard deviation (effect size=2.0). For clinical association studies, please refer to supplementary methods.

Details included in the Supplementary Methods. Mouse models and tumor dissociation; intravital imaging; bioluminescence imaging; blood collection and CTC analysis; invasive cell collection in vivo; cell culture and transfections; CD44 knockout using CRISPR-Cas9 technology; flow cytometry and cell sorting; cell clustering assay; mammosphere assay; lung imaging; RNA extraction and real-time PCR; mass spectrometry; anoikis assay; western blotting; immunohistochemistry; immunofluorescence; clinical outcome association analysis; statistical analysis; and data availability.

### REFERENCES

1. Poste G, Fidler I J. The pathogenesis of cancer metastasis. Nature 1980; 283(5743): 139-46.
2. Chambers A F, Groom A C, MacDonald I C. Dissemination and growth of cancer cells in metastatic sites. Nature reviews 2002; 2(8):563-72 doi 10.1038/nrc865.
3. Gupta G P, Massague J. Cancer metastasis: building a framework. Cell 2006; 127(4):679-95 doi 10.1016/j.cell.2006.11.001.
4. Mu Z, Wang C, Ye Z, Austin L, Civan J, Hyslop T, et al. Prospective assessment of the prognostic value of circulating tumor cells and their clusters in patients with advanced-stage breast cancer. Breast Cancer Res Treat 2015; 154(3):563-71 doi 10.1007/s10549-015-3636-4.
5. Aceto N, Bardia A, Miyamoto D T, Donaldson M C, Winner B S, Spencer J A, et al. Circulating tumor cell clusters are oligoclonal precursors of breast cancer metastasis. Cell 2014; 158(5):1110-22 doi S0092-8674(14)00927-1 [pii] 10.1016/j.cell.2014.07.013.
6. Bonnet D, Dick J E. Human acute myeloid leukemia is organized as a hierarchy that originates from a primitive hematopoietic cell. Nat Med 1997; 3(7):730-7.
7. Kreso A, Dick J E. Evolution of the cancer stem cell model. Cell Stem Cell 2014; 14(3):275-91 doi 10.1016/j.stem.2014.02.006.
8. Lapidot T, Sirard C, Vormoor J, Murdoch B, Hoang T, Caceres-Cortes J, et al. A cell initiating human acute myeloid leukaemia after transplantation into SCID mice. Nature 1994; 367(6464):645-8 doi 10.1038/367645a0.
9. Al-Hajj M, Wicha M S, Benito-Hernandez A, Morrison S J, Clarke M F. Prospective identification of tumorigenic breast cancer cells. Proc Natl Acad Sci USA 2003; 100(7):3983-8.
10. Diehn M, Cho R W, Lobo N A, Kalisky T, Dorie M J, Kulp A N, et al. Association of reactive oxygen species levels and radioresistance in cancer stem cells. Nature 2009; 458(7239):780-3.
11. Chao M P, Alizadeh A A, Tang C, Myklebust J H, Varghese B, Gill S, et al. Anti-CD47 antibody synergizes with rituximab to promote phagocytosis and eradicate non-Hodgkin lymphoma. Cell 2010; 142(5):699-713 doi S0092-8674(10)00892-5 [pii] 10.1016/j.cell.2010.07.044.
12. Jaiswal S, Jamieson C H, Pang W W, Park C Y, Chao M P, Majeti R, et al. CD47 is upregulated on circulating hematopoietic stem cells and leukemia cells to avoid phagocytosis. Cell 2009; 138(2):271-85 doi S0092-8674(09)00651-5 [pii] 10.1016/j.cell.2009.05.046.
13. Bao S, Wu Q, McLendon R E, Hao Y, Shi Q, Hjelmeland A B, et al. Glioma stem cells promote radioresistance by preferential activation of the DNA damage response. Nature 2006; 444(7120):756-60.
14. Eyler C E, Rich J N. Survival of the fittest: cancer stem cells in therapeutic resistance and angiogenesis. J Clin Oncol 2008; 26(17):2839-45.
15. Chen J, Li Y, Yu T S, McKay R M, Burns D K, Kernie S G, et al. A restricted cell population propagates glioblastoma growth after chemotherapy. Nature 2012; 488(7412):522-6 doi 10.1038/nature11287.
16. Kurtova A V, Xiao J, Mo Q, Pazhanisamy S, Krasnow R, Lerner S P, et al. Blocking PGE2-induced tumour repopulation abrogates bladder cancer chemoresistance. Nature 2015; 517(7533):209-13 doi 10.1038/nature14034.
17. Liu H, Patel M R, Prescher J A, Patsialou A, Qian D, Lin J, et al. Cancer stem cells from human breast tumors are involved in spontaneous metastases in orthotopic mouse models. Proc Natl Acad Sci USA 2010; 107(42):18115-20 doi 1006732107 [pii] 10.1073/pnas.1006732107.
18. Godar S, Ince T A, Bell G W, Feldser D, Donaher J L, Bergh J, et al. Growth-inhibitory and tumor-suppressive functions of p53 depend on its repression of CD44 expression. Cell 2008; 134(1):62-73 doi 10.1016/j.cell.2008.06.006.
19. Mani S A, Guo W, Liao M J, Eaton E N, Ayyanan A, Zhou A Y, et al. The epithelial-mesenchymal transition generates cells with properties of stem cells. Cell 2008; 133(4):704-15 doi 10.1016/j.cell.2008.03.027.
20. Dalerba P, Dylla S J, Park I K, Liu R, Wang X, Cho R W, et al. Phenotypic characterization of human colorectal cancer stem cells. Proc Natl Acad Sci USA 2007; 104(24):10158-63 doi 10.1073/pnas.0703478104.
21. Prince M E, Sivanandan R, Kaczorowski A, Wolf G T, Kaplan M J, Dalerba P, et al. Identification of a subpopulation of cells with cancer stem cell properties in head and neck squamous cell carcinoma. Proc Natl Acad Sci USA 2007; 104(3):973-8 doi 10.1073/pnas.0610117104.
22. Li C, Heidt D G, Dalerba P, Burant C F, Zhang L, Adsay V, et al. Identification of pancreatic cancer stem cells. Cancer research 2007; 67(3):1030-7 doi 10.1158/0008-5472.CAN-06-2030.
23. Bockhorn J, Prat A, Chang Y F, Liu X, Huang S, Shang M, et al. Differentiation and loss of malignant character of spontaneous pulmonary metastases in patient-derived breast cancer models. Cancer research 2014; 74(24):7406-17 doi 10.1158/0008-5472.CAN-14-1188.
24. Patsialou A, Bravo-Cordero J J, Wang Y, Entenberg D, Liu H, Clarke M, et al. Intravital multiphoton imaging reveals multicellular streaming as a crucial component of in vivo cell migration in human breast tumors. Intravital 2013; 2(2):e25294 doi 10.4161/intv.25294.
25. Harney A S, Arwert E N, Entenberg D, Wang Y, Guo P, Qian B Z, et al. Real-Time Imaging Reveals Local, Transient Vascular Permeability, and Tumor Cell Intravasation Stimulated by TIE2hi Macrophage-Derived VEGFA. Cancer discovery 2015; 5(9):932-43 doi 10.1158/2159-8290.CD-15-0012.
26. Kumar A, Molli P R, Pakala S B, Bui Nguyen T M, Rayala S K, Kumar R. PAK thread from amoeba to mammals. Journal of cellular biochemistry 2009; 107(4):579-85 doi 10.1002/jcb.22159.
27. Alix-Panabieres C, Pantel K. Challenges in circulating tumour cell research. Nature reviews 2014; 14(9):623-31 doi 10.1038/nrc3820.
28. Cheung K J, Padmanaban V, Silvestri V, Schipper K, Cohen J D, Fairchild A N, et al. Polyclonal breast cancer metastases arise from collective dissemination of keratin 14-expressing tumor cell clusters. Proc Natl Acad Sci USA 2016; 113(7):E854-63 doi 10.1073/pnas.1508541113.
29. Wyckoff J B, Wang Y, Lin E Y, Li J F, Goswami S, Stanley E R, et al. Direct visualization of macrophage-assisted tumor cell intravasation in mammary tumors. Cancer Res 2007; 67(6):2649-56 doi 10.1158/0008-5472.CAN-06-1823.
30. Williams J K, Entenberg D, Wang Y, Avivar-Valderas A, Padgen M, Clark A, et al. Validation of a device for the active manipulation of the tumor microenvironment during intravital imaging. Intravital 2016; 5(2) doi 10.1080/21659087.2016.1182271.
31. Yu M, Bardia A, Winner B S, Stott S L, Smas M E, Ting D T, et al. Circulating breast tumor cells exhibit dynamic changes in epithelial and mesenchymal composition. Science 2013; 339(6119):580-4 doi 10.1126/science.1228522.
32. Ramos E K, Hoffmann A D, Gerson S L, Liu H. New Opportunities and Challenges to Defeat Cancer Stem Cells. Trends Cancer 2017; 3(11):780-96 doi 10.1016/j.trecan.2017.08.007.
33. Dashzeveg N K, Taftaf R, Ramos E K, Torre-Healy L, Chumakova A, Silver D J, et al. New Advances and Challenges of Targeting Cancer Stem Cells. Cancer Res 2017; 77(19):5222-7 doi 10.1158/0008-5472.CAN-17-0054.
34. Vassilopoulos A, Chisholm C, Lahusen T, Zheng H, Deng C X. A critical role of CD29 and CD49f in mediating metastasis for cancer-initiating cells isolated from a Brca1-associated mouse model of breast cancer. Oncogene 2014; 33(47):5477-82 doi 10.1038/onc.2013.516.
35. Sanjana N E, Shalem O, Zhang F. Improved vectors and genome-wide libraries for CRISPR screening. Nature methods 2014; 11(8):783-4 doi 10.1038/nmeth.3047.
36. Ponta H, Sherman L, Herrlich P A. CD44: from adhesion molecules to signalling regulators. Nature reviews Molecular cell biology 2003; 4(1):33-45.
37. Toole B P. Hyaluronan: from extracellular glue to pericellular cue. Nature reviews 2004; 4(7):528-39 doi 10.1038/nrc1391.
38. Slomiany M G, Dai L, Tolliver L B, Grass G D, Zeng Y, Toole B P. Inhibition of Functional Hyaluronan-CD44 Interactions in CD133-positive Primary Human Ovarian Carcinoma Cells by Small Hyaluronan Oligosaccharides. Clin Cancer Res 2009; 15(24):7593-601 doi 10.1158/1078-0432.CCR-09-2317.
39. Kolev V N, Tam W F, Wright Q G, McDermott S P, Vidal C M, Shapiro I M, et al. Inhibition of FAK kinase activity preferentially targets cancer stem cells. Oncotarget 2017; 8(31):51733-47 doi 10.18632/oncotarget.18517.
40. Golubovskaya V M. FAK and Nanog cross talk with p53 in cancer stem cells. Anticancer Agents Med Chem 2013; 13(4):576-80.
41. Mizuno H, Kitada K, Nakai K, Sarai A. PrognoScan: a new database for meta-analysis of the prognostic value of genes. BMC medical genomics 2009; 2:18 doi 10.1186/1755-8794-2-18.
42. van der Toom E E, Verdone J E, Gorin M A, Pienta K J. Technical challenges in the isolation and analysis of circulating tumor cells. Oncotarget 2016 doi 10.18632/oncotarget.11191.
43. Yu M, Stott S, Toner M, Maheswaran S, Haber D A. Circulating tumor cells: approaches to isolation and characterization. The Journal of cell biology 2011; 192(3):373-82 doi 10.1083/jcb.201010021.
44. Warkiani M E, Khoo B L, Wu L, Tay A K, Bhagat A A, Han J, et al. Ultra-fast, label-free isolation of circulating tumor cells from blood using spiral microfluidics. Nature protocols 2016; 11(1):134-48 doi 10.1038/nprot.2016.003.
45. Karabacak N M, Spuhler P S, Fachin F, Lim E J, Pai V, Ozkumur E, et al. Microfluidic, marker-free isolation of circulating tumor cells from blood samples. Nature protocols 2014; 9(3):694-710 doi 10.1038/nprot.2014.044.
46. Maddipati R, Stanger B Z. Pancreatic Cancer Metastases Harbor Evidence of Polyclonality. Cancer discovery 2015; 5(10):1086-97 doi 10.1158/2159-8290.CD-15-0120.
47. Salhia B, Trippel M, Pfaltz K, Cihoric N, Grogg A, Ladrach C, et al. High tumor budding stratifies breast cancer with metastatic properties. Breast Cancer Res Treat 2015; 150(2):363-71 doi 10.1007/s10549-015-3333-3.
48. Au S H, Storey B D, Moore J C, Tang Q, Chen Y L, Javaid S, et al. Clusters of circulating tumor cells traverse capillary-sized vessels. Proc Natl Acad Sci USA 2016; 113(18):4947-52 doi 10.1073/pnas.1524448113.
49. Perrais M, Chen X, Perez-Moreno M, Gumbiner B M. E-cadherin homophilic ligation inhibits cell growth and epidermal growth factor receptor signaling independently of other cell interactions. Mol Biol Cell 2007; 18(6):2013-25 doi 10.1091/mbc. e06-04-0348.
50. Muller W A, Weigl S A, Deng X, Phillips D M. PECAM-1 is required for transendothelial migration of leukocytes. J Exp Med 1993; 178(2):449-60.
51. Peach R J, Hollenbaugh D, Stamenkovic I, Aruffo A. Identification of hyaluronic acid binding sites in the extracellular domain of CD44. The Journal of cell biology 1993; 122(1):257-64.
52. Marlin J W, Chang Y W, Ober M, Handy A, Xu W, Jakobi R. Functional PAK-2 knockout and replacement with a caspase cleavage-deficient mutant in mice reveals differential requirements of full-length PAK-2 and caspase-activated PAK-2p34. Mamm Genome 2011; 22(5-6):306-17 doi 10.1007/s00335-011-9326-6.
53. Reddy P N, Radu M, Xu K, Wood J, Harris C E, Chemoff J, et al. p21-activated kinase 2 regulates HSPC cytoskeleton, migration, and homing via CDC42 activation and interaction with beta-Pix. Blood 2016; 127(16):1967-75 doi 10.1182/blood-2016-01-693572.
54. Rudel T, Bokoch G M. Membrane and morphological changes in apoptotic cells regulated by caspase-mediated activation of PAK2. Science (New York, N.Y. 1997; 276(5318):1571-4.
55. Zhang Y, Wester L, He J, Geiger T, Moerkens M, Siddappa R, et al. IGF1R signaling drives antiestrogen resistance through PAK2/PIX activation in luminal breast cancer. Oncogene 2018 doi 10.1038/s41388-017-0027-9.
56. Sarioglu A F, Aceto N, Kojic N, Donaldson M C, Zeinali M, Hamza B, et al. A microfluidic device for label-free, physical capture of circulating tumor cell clusters. Nature methods 2015; 12(7):685-91 doi 10.1038/nmeth.3404.
57. Labelle M, Begum S, Hynes R O. Direct signaling between platelets and cancer cells induces an epithelial-mesenchymal-like transition and promotes metastasis. Cancer Cell 2011; 20(5):576-90 doi 10.1016/j.ccr.2011.09.009.
58. Yang J, Yan R, Roy A, Xu D, Poisson J, Zhang Y. The I-TASSER Suite: protein structure and function prediction. Nat Methods 2015; 12(1):7-8 doi 10.1038/nmeth.3213.
59. Kozakov D, Hall D R, Xia B, Porter K A, Padhorny D, Yueh C, et al. The ClusPro web server for protein-protein docking. Nat Protoc 2017; 12(2):255-78 doi 10.1038/nprot.2016.169.
60. Chen H, Sun Y, Shen Y. Predicting protein conformational changes for unbound and homology docking: learning from intrinsic and induced flexibility. Proteins 2017; 85(3):544-56 doi 10.1002/prot.25212.
61. Oliwa T, Shen Y. cNMA: a framework of encounter complex-based normal mode analysis to model conformational changes in protein interactions. Bioinformatics 2015; 31(12):i151-60 doi 10.1093/bioinformatics/btv252.
62. Alexander N, Woetzel N, Meiler J. bcl::Cluster: A method for clustering biological molecules coupled with visualization in the Pymol Molecular Graphics System. IEEE Int Conf Comput Adv Bio Med Sci 2011; 2011:13-8 doi 10.1109/ICCABS.2011.5729867.
63. Brown W. Some experimental results in the correlation of mental abilities. British Journal of Psychology 1910; 3:296-322.
64. Spearman C C. Correlation calculated from faulty data. British Journal of Psychology 1910; 3:271-95.

Supplementary Methods and Materials

Mouse models and tumor dissociation. NOD/SCID mice 8-10 weeks of age were used for PDXs and human MDA-MB-231 cell-based xenograft studies. The triple negative (TN) PDXs and MDA-MB-231 cells were lentivirally labeled by eGFP, tdTomato, Luc2-eGFP (L2G), or Luc2-tdTomato (L2T) using the lentiviruses and labeling protocol previously described (1,2). PyMT (MMTV-PyMT) transgenic mice (3) were bred and crossed with MacBlue mice [Csflr-GAL4-VP16/UAS-enhanced cyan fluorescent protein (ECFP)](4) in the animal facility of Albert Einstein College of Medicine.

PDX tumors were harvested and dissociated either with collagenase III (TN1 model) or liberase TH and TM research grade enzyme blends (TN2 model and lung tissues). Briefly, tumors or lung tissues were minced and incubated for 2-4 h at 37° C. with collagenase III (Worthington Biochemical) or liberase TH and TM (Roche) and 100 Kunitz U of DNase I (Sigma) in 20 mL of RPMI medium with 20 mM HEPES buffer. Single-cell suspensions were filtered through 40-μm nylon cell strainers and washed with Hank's balanced salt solution (HBSS; Sigma) containing 2% heat-inactivated fetal bovine serum (FBS). Red blood cells were lysed with ACK lysis buffer, and dissociated bulk tumor cells were either cultured or stained with various antibodies in HBSS/2% FBS for further flow analysis or sorting on a BD FacsAria (BD Biosciences). 4′,6-diamidino-2-phenylindole (DAPI) and H2Kd were used as markers for viability and mouse stromal cells, respectively.

Intravital imaging. For PyMT mice, the multiphoton intravital imaging was performed using a skin flap procedure as previously described with a custom-built 2-laser multiphoton microscope (3,5). The animal was placed in a heated chamber maintained at physiologic temperature during the course of imaging and monitored using MouseOx (Starr Life Sciences). In order to label the blood vessels, three milligrams of 155-kDa TMR-dextran or 100 μL of 8 μmol/L Qdots 705 was administered via a tail vein catheter (Qdots [Qdots ITK 705] were obtained from Life Technologies or A. Smith, University of Illinois Urbana-Champaign).

For TN PDX models, the intravital imaging was performed on tumors that had reached 0.7-1 cm in diameter as the optimal time window, using an Olympus FV1000-MPE microscope with a 25×, 1.05 NA water immersion objective with correction collar as described (6). The laser-light source consists of a standard femtosecond-pulsed laser system (Mai Tai HP with DeepSee, Newport/Spectra-Physics). Texas Red 2 dextran (70 kDa; Invitrogen, cat #D1830) was used to mark the blood vasculature through the lateral tail veins of the mice, just prior to an imaging session. In vivo migration images were collected in random fields of 512×512 μm at 512×512 pixels for a depth of 100 μm (21 slices at steps of 5 μm) beginning at the edge of the tumor. Images were taken at 2 min intervals for a total of 30 min. TN1 cells analyzed in this study expressed eGFP (L2G) and were visualized based on their fluorescence expression. Images were reconstructed in 3D and through time using ImageJ.

Intravital multiphoton imaging of MDA-MB-231 tumor-bearing mice was performed with methods similar to previous studies (6) using an Olympus FV1000-MPE microscope with a 25×, 1.05 NA water immersion objective with correction collar. Imaging was performed on fields of 512×512 μm at 512×512 pixels for a depth of 100 μm (21 slices at steps of 5 μm) beginning at the edge of the tumor. The edge of the tumor was defined as the junction of the GFP-labeled tumor cells with the absence of GFP signal. Collagen I fibers were captured using the second harmonic signal excited at 880 nm and imaged through a 410-440 nm bandpass filter. Images were acquired at 2 min intervals for a total of 30 min. Blood vessels were visualized by direct injection of Texas Red dextran (Invitrogen) through the lateral tail veins of the mice just prior to intravital imaging.

Bioluminescence imaging. Mice were injected intraperitoneally with 100 μL of D-luciferin (30 mg/mL, Gold Biotechnology). After 5-10 min, mice were anesthetized with isoflurane, and bioluminescence images were acquired using the Xenogen IVIS spectrum system (Caliper Life Sciences). Acquisition times ranged from 5 s to 5 min. Signals are presented as total photon flux and analyzed using Living Image 3.0 software (Caliper Life Sciences).

Blood collection and CTC analysis. This study was approved by the Institutional Review Board (IRB) of Northwestern University. All patients signed informed consent to participate in this study. Approximately 8-10 mL of whole blood was collected from breast cancer patients into a 10-mL CellSave Preservative tube containing a cellular fixative (Janssen Diagnostics, LLC, Raritan, N.J.). Blood specimens were maintained at room temperature (RT) and processed within 96 h of being drawn. CTC analysis was performed using CellSearch® CTC kits on the FDA-approved CellSearch System (Janssen Diagnostics), as previously described (7). CTCs were identified by positive staining for both cytokeratins (CK) and DAPI and negative staining for CD45 (CK+/DAPI+/CD45−). CTC clusters were defined as an aggregation of two or more individual CTCs containing distinct nuclei and intact cytoplasmic membranes. To determine the expression of CD44 on CTCs, the FITC-conjugated anti-CD44 antibody (BD) was also added.

For the PDXs, after tumors reached about 2 cm in diameter, up to 1 mL fresh whole blood was collected via terminal cardiac puncture of the right ventricle using a 25G or 26G needle attached to a 1 mL syringe prefilled with 100 μL phosphate-buffered saline (PBS)/EDTA, and rapidly injected to a falcon tube with an equal volume of PBS/10 mM EDTA. Red blood cells were optionally depleted with 2% (weight/volume) dextran incubation for 30 min at 37° C. Unsettled cells were spun and followed by red blood cell lysis with ACK buffer (Invitrogen). The rest of the cells were stained with 1 μg/mL Hoechst 33342 (optional) and spread onto a 24-well plate, and live CTCs were imaged under fluorescence microscopy (Nikon).

Invasive cell collection in vivo. Micro-needle collection of breast tumor cells in live anesthetized mice was carried out as described previously (8,9). Human recombinant EGF (Invitrogen) (25 nmol/L) was used as a chemoattractant for active collection. Cells can only be collected into the needles by active migration and invasion because a Matrigel block is used to prevent passive collection of cells and tissue during insertion of the needle into the tissue. After 4 h, the needles were removed from the xenograft tumors and the total number of cells collected was determined by DAPI staining and microscopy analysis.

Hyaluronan inhibition. Two complementary approaches were utilized to inhibit hyaluronan, the known CD44 ligand, in the two models. First, for the TN PDX model, dissociated cells were treated with hyaluronan antagonist (o-HA, HA oligomers) at 100 μg/mL during the 72-hour aggregation assays. For MDA-MB-231 cells, the hyaluronic acid synthase inhibitor 4-methylumbelliferone (4-MU, 0.4 mM/L) was added to the adherent culture. After 48 h of treatment, the cells were trypsinized and transferred to a poly-hydroxyethyl methacrylate (Poly-HEMA, Sigma-Aldrich) coated plate, and the images were taken at the indicated times by Leica microscopy.

CD44 knockout using CRISPR-Cas9 technology. CRISPR/Cas9 targeting was performed using the LentiCrisprV2 system (10). Guides to knock out CD44 were selected using the online sgRNA analysis tool located at crispr.mit.edu. High ranking guides (>80) in the first three exons that had an in-frame PAM sequence were selected and cloned as previously described into LentiCrisprV2. Following sequence verification, virus was produced by transfection of the LentiCrisprV2 construct, PsPax, and pMD2 in a 1:0.75:0.3 ratio into HEK293T cells. Two days after transfection, supernatants containing virus were harvested, passed through a 0.45 μm filter, and incubated with recipient cells for two days before initiation of puromycin selection. The vector and virus with gRNA1 targeting CD44 exon 2 (F—CACCG TCGCTACAGCATCTCTCGGA; R—AAAC TCCGAGAGATGCTGTAGCGA C) was used in most of the knockout experiments.

Flow cytometry and cell sorting. Dissociated tumor cells from PDXs or cultured MDA-MB-231 cells were resuspended at 10 million per mL in PBS/2% FBS or HBSS/2% FBS. Cells were blocked with IgG prior to incubation with specific antibodies, such as mouse anti-human CD44-APC (BD #559942), isotype control mouse IgG2b-APC (BD #555745), isotype control mouse IgG2b-PE (BD #555743), and for PDXs, the mouse stromal cell marker anti-H2Kd (BD), for 20 min at 4° C., followed by washing twice with PBS. Finally, the cells were diluted in PBS and analyzed on a BD-LSR II flow cytometer (BD Biosciences). Sterile cell preparations were filtered prior to flow analyses, with indicated populations sorted on a BDAria cell sorter (BD Biosciences) and collected in HBSS/20% FCS.

Cell clustering assay. Freshly dissociated primary tumor cells in single cell suspension were seeded in collagen type I-coated 96-well plates. The plates were put into the IncuCyte live cell imaging system (Essen BioScience), and live images were taken every 2-4 h for up to one week. The cluster number and size were analyzed by Incucyte ZOOM software (Essen BioScience). In specific experiments, primary tumor cells might be sorted based on the expression of CD44 prior to seeding. In other experiments, seeded tumor cells might be transfected with siRNAs (100 nM) or treated with various inhibitors during the clustering assays. For cell viability analysis during clustering, the IncuCyte Cytotox Red reagent (Essen BioScience) was added to the medium according to the manufacturer's instructions. For MDA-MB-231 cell-mediated clustering assays, cells were trypsinized into single cell suspension and transferred to poly-hydroxyethyl methacrylate (Poly-HEMA, Sigma-Aldrich)-coated plates. In anti-CD44 blocking experiments, cells were pretreated with IgG control or anti-CD44 antibody (400 μg/ml) for 30 mins, and then transferred to Poly-HEMA-coated plates. For overexpression experiments, HEK-293 cells were transfected with pCMV6-Flag-CD44 or pCMV6-Flag-ΔN21-97 CD44 plasmids for 48 h, and then trypsinized and incubated on the Poly-HEMA coated dishes. Images were taken at the indicated times within 60 min or 24 h by Leica microscopy. For gene modulations, cells were first transfected with siRNAs (100 nM). After 48 h, the cells were then trypsinized prior to clustering assays.

Mammosphere assay. Freshly isolated primary tumor cells were cultured overnight, and then clustered cells were collected by gentle pipetting and centrifugation at 400 rpm for 2 min. One half of the clustered cells were further dissociated by a quick trypsinization into single cells. Then 250 single cells or clusters containing an estimated 250 cells were plated in 96-well tissue culture plates covered with poly-HEMA in PRIME-XV® Tumorsphere serum-free medium (IrvineScientific). After 10 days of culture, the number of spheres with diameter >50 μm was counted.

Lung imaging. For spontaneous lung metastatic foci imaging from orthotopic breast tumor models, 1-5×105 eGFP-TN1 and 1-5×105 dTomato-TN1 tumor cells were prepared separately or mixed 1:1 and injected orthotopically into NOD/SCID mouse mammary fat pads (along with an equal volume of Matrigel from BD). After 8-12 weeks, the lungs were removed and the metastatic foci (single or mixed color) were captured and counted by two-photon or confocal microscopy. For assessment of the metastatic potential single CTCs and CTC clusters, freshly isolated primary tumor cells were cultured overnight, and then cells were collected by gentle pipetting and centrifugation at 400 rpm for 2 min (clustered cells). Half of the clustered cells were further dissociated by a quick trypsinization into single cells. Then 1×106 single cells or clustered equivalent were injected into NOD/SCID mice via tail vein. The mice were euthanized after 2 and 24 h, and lung were removed and imaged by fluorescence microscopy.

For the MDA-MB-231 cell-mediated colonization experiment, 5×105 L2T-labeled and 5×105 L2G-labeled cells were co-injected or separately injected (5 min apart, 10 min apart, and 2 h apart) into NOD/SCID mice via the tail vein. At the indicated times post-injection, the lungs were removed and imaged by fluorescence microscopy. To quantify the single and clustered colonies or the single- and mixed-color colonies in the lung, five or more images of the lungs were taken, and the number per image was counted.

For anti-CD44 blocking experiment, freshly isolated primary tumor cells (TN1 PDXs) were pretreated with IgG control or anti-CD44 antibody (400 μg/ml) for 30 mins, and then cultured in collagen type I-coated plates in the presence of antibody. NOD/SCID or NSG mice were treated (i.p.) with IgG or anti-CD44 antibody (100 μg/mouse) 6 hours before tumor injection via tail vein. For HEK-293 cells, CD44s-FLAG and ΔN21-97-FLAG were overexpressed via transient transfection 48 hours prior to collection and 5×105 cells were subsequent injected into each of the recipient NSG mice via tail vein infusion. The mice were euthanized 24 h post tail vein injection, and lung were removed and imaged by fluorescence microscopy. Five or more images of the lungs per mouse were taken, and the number of tumor cell clusters per image was counted.

RNA extraction and real-time PCR. Total RNAs were extracted using Trizol (Invitrogen), and RNA was precipitated with isopropanol and glycogen (Invitrogen). After reverse transcription reactions, real-time PCR for miRNAs/genes was performed using individual miRNA/gene Taqman primers (Applied Biosystems) with an ABI 7500 real-time PCR system. RNU44 and U6 primers were used for miRNA internal controls and GAPDH for a housekeeping gene control.

To identify the CD44 variants, total RNAs of TN1 and TN2 tumors were isolated using Trizol, and cDNAs were synthesized using gScript™ cDNA SuperMix (Bio-Rad). Real-time PCR was performed on an ABI 7500 system with iQ SYBR Green Supermix (Bio-Rad). The primer sequences were: CD44v3 forward primer, 5′-GCAGGCTGGGAG CCAAAT-3′; and reverse primer, 5′-GAGGTGTCTGTCTCTTT CATCTTCATT-3; CD44v6 forward primer, 5′-GGAACAGTGGTTTGGCAACAG-3′; and reverse primer, 5′-TTGGGTGTTTGGCGATATCC-3′. Results were analyzed with ABI Sequence Detection Software and the PCR products were also visualized in a 2% agarose gel stained with ethidium bromide. GAPDH was used as the housekeeping gene control.

Mass spectrometry. Tumor cell pellets were collected from cell sorting runs or siRNA transfections and then lysed with 2% SDS and protease inhibitor cocktail. Proteins were extracted using pulse sonication, and cleaned up by filter-aided sample preparation (FASP) to remove detergents. After LysC/Trypsin digestion, 500 ng proteins were analyzed via a 4-h LC/MS/MS method at Case Western Proteomics Core facility and the data processed using Scaffold. The fold change was calculated based on total unique spectrum counts.

Solid-phase homophilic interaction assay. High binding EIA/RIA microplate (Corning) were coated with purified CD44 protein (extracellular domain) (Thermo Scientific, 1 μg/well), or bovine serum albumin (BSA; 1 μg/well) in TBS (pH 7.4) overnight, and then blocked with 5% BSA/TBS for 2 h at room temperature. Different concentration of biotin-labeled CD44 (EZ-Link Sulfo-NHS-LC-Biotinylation Kit, Thermo Scientific) in binding buffer (TBS, 0.1% BSA, 1 mM MgCl2, 1 mM CaCl2) was added into coated wells, and incubated for 2 h at room temperature. Bound biotin-labeled CD44 was detected with streptavidin-HRP (Thermo Scientific) and quantified using the tetramethylbenzidine (TMB) substrate reagent kit (Pierce) at 450 nm.

Anoikis assay. Poly-HEMA was reconstituted in 95% ethanol to a concentration of 20 mg/mL. To prepare poly-HEMA-coated plates, 150 μL of poly-HEMA solution was added to each well of a 24-well plate and allowed to dry overnight in a laminar flow tissue culture hood. Cells were transfected and plated in triplicate in poly-HEMA-coated 24-well plates using regular culture medium. After 48 h, cells were collected and apoptosis was assayed by annexin V staining (BD Biosciences) according to the manufacturer's instructions.

Western blotting. Cells were washed twice in cold PBS, and then lysed in RIPA buffer with protein inhibitor cocktail (Sigma-Aldrich) or a buffer containing 50 mM Tris-HCl pH 7.4, 1% NP-40, 0.25% sodium deoxycholate, 150 mM NaCl, 1 mM EDTA, 1 mM NaF, 2 mM Na3VO4, 1 mM PMSF, 10 μg/mL aprotinin and 10 μg/mL leupeptin at 4° C. for 30 min. Equal amounts of protein of each sample were run on an SDS-PAGE gel, transferred to PVDF or Nitrocellulose membranes, blocked with 2% BSA/PBS for 1 h at RT, and then incubated with primary antibodies for 1 h at RT or 4° C. overnight and horseradish peroxidase (HRP)-conjugated secondary antibodies for 1 h at RT. The primary antibodies that were used in our experiments include CD44 (Thermo Fisher Scientific; 156-3C11), PAK2 (Thermo Fisher Scientific, MA5-15527), FAK (Cell Signaling Technology, #3285), Oct 4 (Santa Cruz, sc-5279), FLAG (Sigma-Aldrich, F7425), HA-probe (Santa Cruz, 12CA5), and β-actin (Sigma, A5441).

Co-immunoprecipitation. For overexpression experiments, the cells were trypsinized after transfections, and incubated on the Poly-HEMA coated dishes for 3 h to aggregate. Aggregated cells were then collected, and lysed in Pierce IP lysis buffer (Thermo Fisher Scientific) with protease inhibitor cocktail at 4° C. for 30 min. One mg protein of each sample was incubated with 5 μg of anti-FLAG antibody (Sigma-Aldrich) or 25 μl of anti-HA magnetic beads (Thermo Fisher Scientific). For anti-FLAG immunoprecipitation, 25 μl of protein A/G plus-agarose (Santa Cruz, sc-2003) were added into samples after 1 h, and then further incubation for overnight at 4° C. Anti-HA magnetic bead-based immunoprecipitation was performed following the manufacturer's instruction. Beads were washed four times with washing buffer (1% Triton-100X in 0.1% TBS-T), and binding proteins were eluted with 0.1 M glycine pH 2-3 for 5-10 minutes and added with same amount of TBS to neutralize.

Immunohistochemistry. Formalin-fixed and paraffin-embedded mouse tissues (mouse xenografts primary tumors, lungs) and human tissues (primary tumors, lung, brain, and liver) were processed and sectioned by routine procedures. Heat-mediated antigen retrieval was used for all staining procedures. The tissues were incubated and stained overnight at 4° C. with different primary antibodies. The primary antibodies that were used include CD44 (Thermo Fisher Scientific, 156-3C11), Cytokeratin (Dako; AE1/AE3 (M3515) or CK34BE12), E-cadherin (BD biosciences; 610181), CD31 (abcam, ab28364), and EpCAM (Thermo Fisher Scientific, MA1-10195). All specimens were counterstained with Hematoxylin. Images of the whole tissue were taken with ScanScope (Aperio). CTCs were identified in the pulmonary vasculatures by tumor cell morphology, size and human cell surface markers. Quantitative analysis of CD44 expression in single CTCs and CTC clusters was calculated by the percentage of CD44 positive stained cells in the total population of single CTCs or CTC clusters.

Immunofluorescence. Cells were cultured in the Poly-HEMA coated plates for 48 h, and then were spun onto Cell-Tak (Corning) coated cover slides. Cells were fixed in 4% paraformaldehyde (PFA) for 10 min. After fixation, cells were permeabilized with 0.25% Triton X-100 in PBS, and then blocked with 2% bovine serum albumin in PBS for 1 h. All primary antibodies were incubated at RT for 1 h or at 4° C. overnight. Cells were then washed 3 times with PBS and incubated with Alexa 488- or Alexa 568-conjugated secondary antibodies (Thermo Fisher Scientific) for 1 h. Nuclei were counterstained with DAPI. The images were taken on a Leica TCS SP8 confocal microscope (Leica Microsystems). The primary antibodies that were used included CD44 (Thermo Fisher Scientific, 156-3C11) and the CellSearch kit (CD45 and CK).

Clinical outcome association analysis. Overall survival of Northwestern University cohort patients was defined as the time between CTC assessment blood draw and death from any cause. Differences in survival were tested by log-rank test and represented by Kaplan-Meier estimator plot. Statistical analysis was performed using STATA (StataCorp. (2015) Stata Statistical Software: Release 14.2. College Station, Tex.: StataCorp LP).

Overall survival, relapse-free survival, and distant metastasis-free survival of breast cancer patient cohorts in publicly or selectively available databases were analyzed via the online program Prognoscan using the optimal cutoff (11). The clinical information chart is referred to in Table 4.

Statistical Analysis

Inclusion and exclusion criteria for samples and animals. Animals with sickness and injury unrelated to implanted tumors were excluded from further studies and data analysis, based on a veterinarian's order. One clinical blood specimen with solely CTC clusters without single CTC counts at the first examination was excluded after the second examination report of zero CTC counts. Randomization of animal groups. Female NOD/SCID or NSG mice were randomized by cages with matched age and weight for different tumor implantations. For treatment groups and controls, equivalent tumor burden was also matched for randomized pre-clinical trials. Tumor growth and treatment response were objectively analyzed by bioluminescence and fluorescence imaging.

Blinded experimental design. One team of investigators performed the tumor implantation and another team was assigned to conduct blinded tumor imaging under non-blinded supervision. The imaging results were analyzed by multiple people in a blinded and non-blinded combination.

Data availability. All of the data included in the manuscript are available to be shared upon request. Information on the public databases of breast tumors is provided in the Table 5.

### REFERENCES

1. Liu H, Patel M R, Prescher J A, Patsialou A, Qian D, Lin J, et al. Cancer stem cells from human breast tumors are involved in spontaneous metastases in orthotopic mouse models. Proc Natl Acad Sci USA 2010; 107(42):18115-20 doi 1006732107 [pii] 10.1073/pnas.1006732107.
2. Bockhorn J, Dalton R, Nwachukwu C, Huang S, Prat A, Yee K, et al. MicroRNA-30c inhibits human breast tumour chemotherapy resistance by regulating TWF1 and IL-11. Nat Commun 2013; 4:1393 doi http://www.nature.com/ncomms/jouirnal/v4/n1/suppinfo/ncomms2393_S1.html.
3. Harney A S, Arwert E N, Entenberg D, Wang Y, Guo P, Qian B Z, et al. Real-Time Imaging Reveals Local, Transient Vascular Permeability, and Tumor Cell Intravasation Stimulated by TIE2hi Macrophage-Derived VEGFA. Cancer Discov 2015; 5(9):932-43 doi 10.1158/2159-8290.CD-15-0012.
4. Ovchinnikov D A, van Zuylen W J, DeBats C E, Alexander K A, Kellie S, Hume D A. Expression of Gal4-dependent transgenes in cells of the mononuclear phagocyte system labeled with enhanced cyan fluorescent protein using Csflr-Gal4VP16/UAS-ECFP double-transgenic mice. Journal of leukocyte biology 2008; 83(2):430-3 doi 10.1189/jlb.0807585.
5. Entenberg D, Wyckoff J, Gligorijevic B, Roussos E T, Verkhusha V V, Pollard J W, et al. Setup and use of a two-laser multiphoton microscope for multichannel intravital fluorescence imaging. Nature protocols 2011; 6(10): 1500-20 doi 10.1038/nprot. 2011.376.
6. Patsialou A, Bravo-Cordero J J, Wang Y, Entenberg D, Liu H, Clarke M, et al. Intravital multiphoton imaging reveals multicellular streaming as a crucial component of in vivo cell migration in human breast tumors. Intravital 2013; 2(2):e25294 doi 10.4161/intv.25294.
7. Mu Z, Wang C, Ye Z, Austin L, Civan J, Hyslop T, et al. Prospective assessment of the prognostic value of circulating tumor cells and their clusters in patients with advanced-stage breast cancer. Breast Cancer Res Treat 2015; 154(3):563-71 doi 10.1007/s10549-015-3636-4.
8. Wyckoff J, Wang W, Lin E Y, Wang Y, Pixley F, Stanley E R, et al. A paracrine loop between tumor cells and macrophages is required for tumor cell migration in mammary tumors. Cancer research 2004; 64(19):7022-9.
9. Wyckoff J B, Segall J E, Condeelis J S. The collection of the motile population of cells from a living tumor. Cancer research 2000; 60(19):5401-4.
10. Sanjana N E, Shalem O, Zhang F. Improved vectors and genome-wide libraries for CRISPR screening. Nature methods 2014; 11(8):783-4 doi 10.1038/nmeth.3047.
11. Mizuno H, Kitada K, Nakai K, Sarai A. PrognoScan: a new database for meta-analysis of the prognostic value of genes. BMC medical genomics 2009; 2:18 doi 10.1186/1755-8794-2-18.

### Example 2—EGFR Promotes CD44-Mediated Breast Tumor Cluster Formation in Metastasis

Abstract

The epidermal growth factor receptor (EGFR) is known to be involved in several cancers, however, it is unclear whether it has a role in promoting circulating tumor cell (CTC) cluster-mediated metastasis. Our previous research demonstrated CD44 as a promoter of CTC clustering, which increases survival and drives metastasis. Our data demonstrates that EGFR contributes to the formation of this cell aggregation in a synergy with CD44. We found an EGFR monoclonal antibody (anti-EGFR, clone LA1, Millipore) that effectively blocks clustering in vitro and reduces lung metastasis. Furthermore, we present miR-30c as a potential therapeutic to disrupt CD44 and EGFR mediated clustering.

Introduction

Metastasis remains as the major cause of cancer mortality and it demands a better understanding for more effective treatments. In order for tumor cells to metastasize, they must overcome several barriers. One of the first a few steps are invasion and intravasation from the primary tumor in order to circulate through the peripheral vasculature. Circulating tumor cells (CTCs) are associated with a poor prognosis. In addition to the dogma of single cell-mediated dissemination, we recently demonstrated that clustered CTCs are more tumorigenic and metastatic than single CTCs with advantages of enhanced regenerative power or stem cell properties (stemness)¹. However, there is no existing therapeutics targeting CTC clusters to our knowledge.

Stemness has been demonstrated to be one of the requisites for successful cancer metastasis¹⁻³. Within a tumor, such subpopulations of cancer cells with regenerative stemness have the potential of self-renewal, proliferation, plasticity, and differentiation, giving rise to heterogeneous progenies^{4,5}. Many molecular markers of stemness have been identified in various cancer types, such as CD44 in breast cancer⁶ and LGR5 in colon cancer^{2,7-10}, whereas the functional contribution of CD44 to stemness and metastasis has been elusive. Notably, our recent studies have unveiled a new role of CD44 in circulating tumor cell cluster aggregation via its homophilic interactions that drive polyclonal metastases¹. However, the regulatory network surrounding CD44's function in CTC clusters and subsequent therapeutic targeting strategies are largely unknown and yet to be determined.

The epidermal growth factor receptor (EGFR) is a tyrosine kinase that has been known to be involved in several cancers by promoting its growth, differentiation and migration¹¹. Several targeted treatments have been developed to target EGFR through tyrosine kinase inhibitors and monoclonal antibodies. FDA approved drugs such as Cetuximab and Erlotinib have proven to be effective in squamous cell carcinoma of the head and neck and lung cancer respectively, however, an effective treatment blocking EGFR in breast cancer remains to be identified¹¹. Although the importance of EGFR in cancer formation is well established, its role in CTC cluster-mediated metastasis and cross talk with CD44 are not well understood. Here, we seek to identify the role of EGFR in breast cancer clustering. We have identified a CD44-targeting microRNA, miR-30c and a monoclonal antibody of EGFR that has a potential efficacy to inhibit breast cancer metastasis.

Results

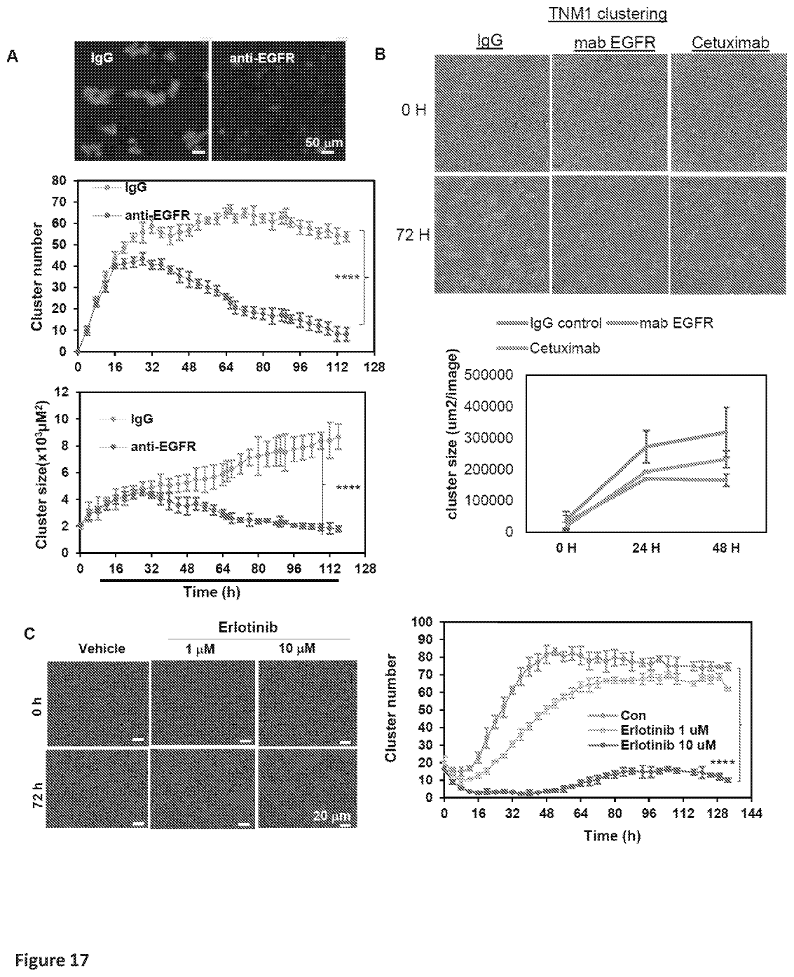

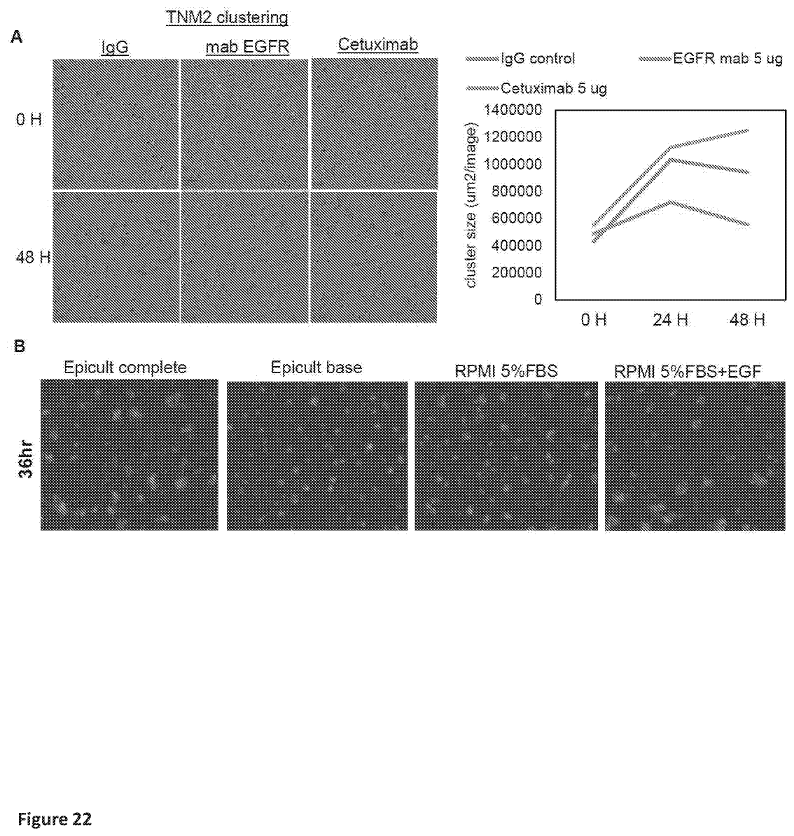

EGFR promotes cell clustering. Through a selected screening of neutralizing antibodies we identified an EGFR monoclonal antibody, clone LA1 instead of Cetuximab, as a strong inhibitor of clustering formation (FIG. 17A-B, FIG. 22A). Additionally, we observed an increased cluster formation when media was supplemented with EGF (FIG. 22B). Consistently, the incubation with Erlotinib (1-10 μM), a kinase inhibitor of EGFR, dramatically reduced PDX-derived tumor cell cluster formation, both the counts and size (FIG. 17C). Hence, we seek to identify whether EGFR activation has a role in clustering formation and observed increased phosphorylation of EGFR (Y845) overtime (FIG. 17D). Taken together, we identified EGFR as a promoter of cell clustering.

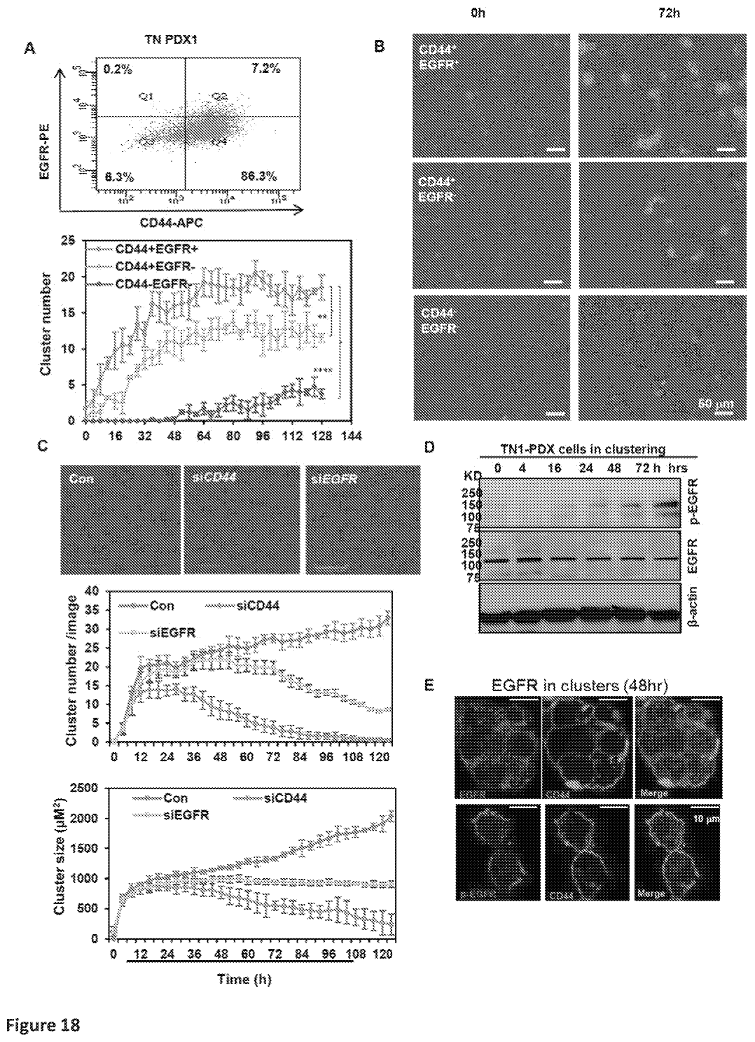

CD44 promotes EGFR stability and activity in clusters. Since we had previously identified CD44 as an essential mediator of CTC cluster formation, we examined if EGFR strengthens CD44 functions in this process. We first observed that EGFR+ PDX tumor cells were all CD44+ (FIG. 18A), and therefore only sorted three subpopulations according to both expression, which were CD44+/EGFR+, CD44+/EGFR− and CD44−/EGFR− cells. The EGFR positivity further improved the cluster formation of CD44+ cells while the double negative cells displayed the lowest efficiency of cluster formation (FIG. 18A-B). Knockdown of EGFR blocked cluster formation, partially mimicked the effects of CD44 knockdown (FIG. 18C).

We then found that during the tumor clustering course, EGFR was phosphorylated (FIG. 18D). Furthermore, pEGFR co-localizes with CD44 in clustered tumor cells shown by immunofluorescence staining (FIG. 18E).

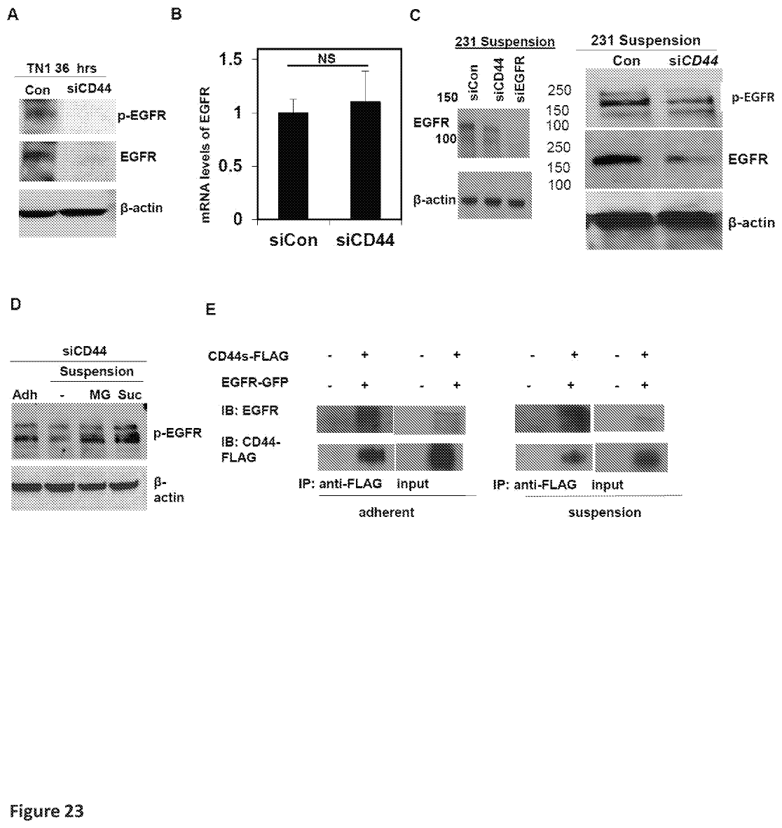

Upon knockdown of CD44 there is a reduction in the total EGFR expression and hence phosphorylation (Y845) in TN1 PDX cells (FIG. 23A). However, there is no transcriptional regulation of EGFR by CD44 as shown by no difference in EGFR mRNA levels upon CD44 knock down by siCD44 transfection (FIG. 23B). Furthermore, CD44 knockdown consistently led to a reduction of the EGFR protein levels in 231 cells (FIG. 23C). We then hypothesized that CD44 helps stabilize EGFR expression. When protein degradation pathways were blocked by proteasome inhibitor MG-132 and endocytosis inhibitor sucrose, CD44 knockdown caused the reduction of p-EGFR levels were rescued (FIG. 23D). Based on the co-immunoprecipitation study using antibodies against CD44-HA, CD44 and EGFR were found to be interacting in the same protein complex (FIG. 23E). Overall, these data suggest that CD44 helps stabilize EGFR from endocytosis-based turnover through protein interactions.

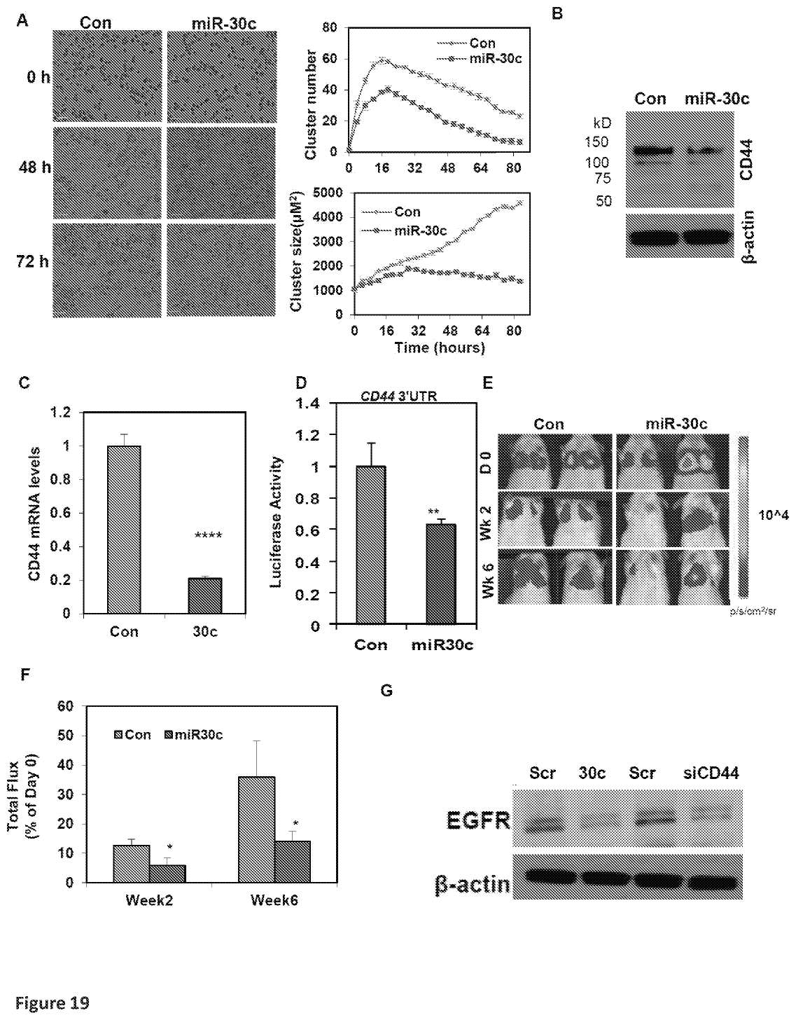

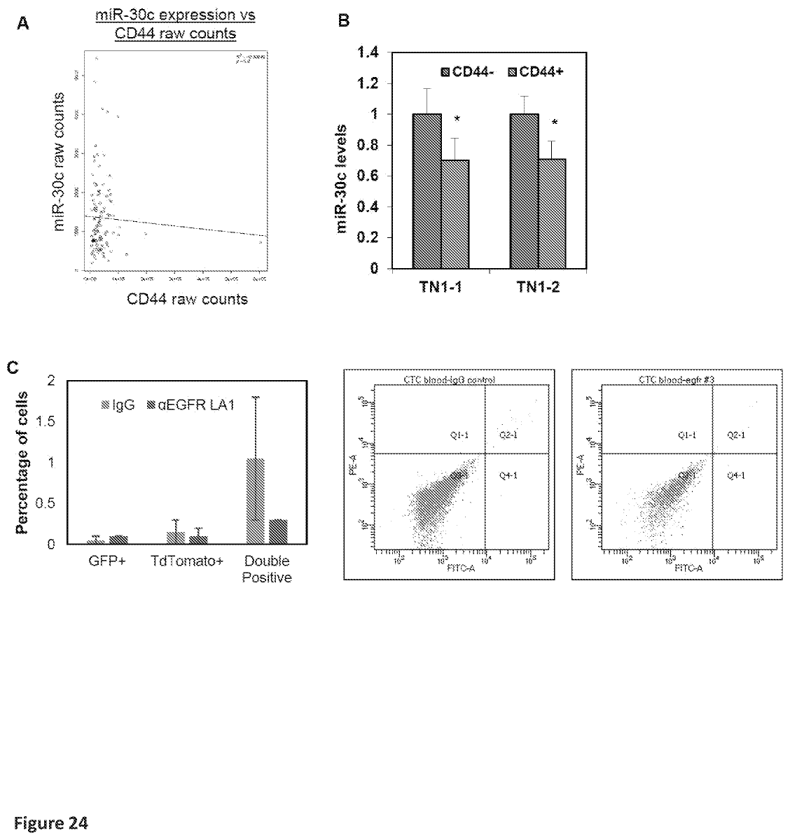

miR-30c reduces cell clustering and metastasis by targeting CD44. In our previous work we observed that microRNA 30c induction in breast cancer cells is effective in reducing metastasis in vivo and to inhibit chemotherapy resistance^{12,13}. Now we further found that overexpression of miR-30c in patient derived xenograft (PDX) models results in a reduction in size and counts of breast tumor cell cluster formation in vitro (FIG. 19A). In breast cancer patients we observed a possible trend (without a significant p value) of negative correlation between miR-30c and CD44 mRNA counts (FIG. 24A). Nevertheless, the expression of miR-30c in CD44 positive cells was lower compared to that of CD44 negative cells in multiple PDX models (FIG. 24B). We therefore tested the hypothesis that miR30c targets CD44. Indeed, miR-30c overexpression caused a reduction in CD44 mRNA and protein expression (FIG. 19B-C). A 3′UTR luciferase assay shows an inhibitory binding of miR-30c to the 3′UTR region of CD44, confirming CD44 as a direct target of miR-30c (FIG. 19D). In vivo studies further demonstrated that miR-30c upregulation reduces lung colonization of TN1 PDX tumor cells upon tail vein infusion (FIG. 19E-F). As a result, miR-30c inhibits both CD44 and EGFR protein levels (FIG. 19G). Overall, miR-30c could serve as an alternative therapeutic approach to prevent CTC clustering and hence metastasis.

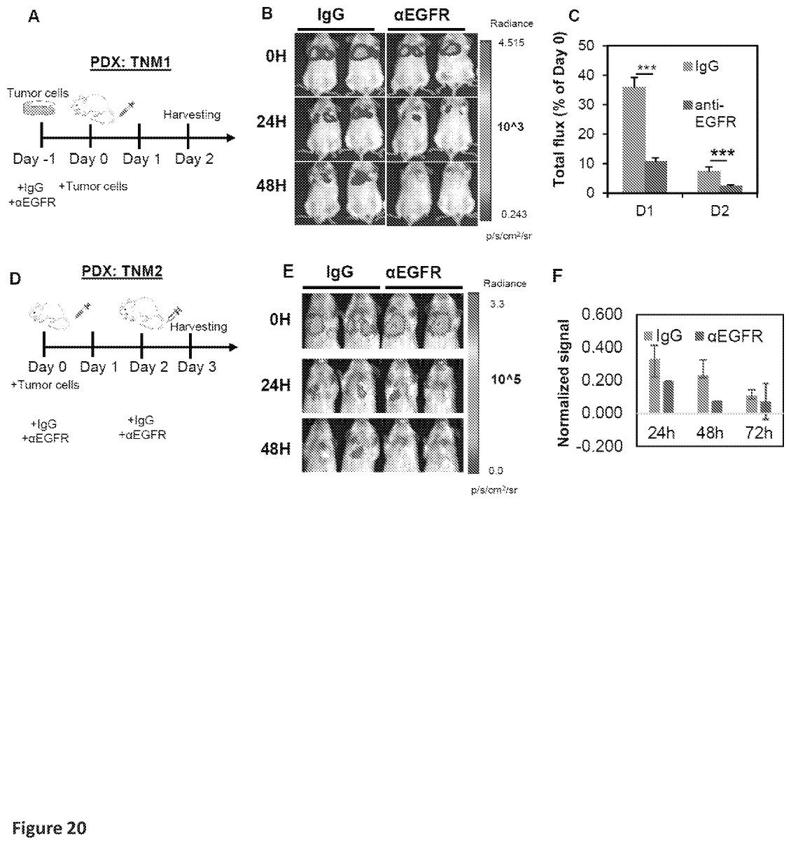

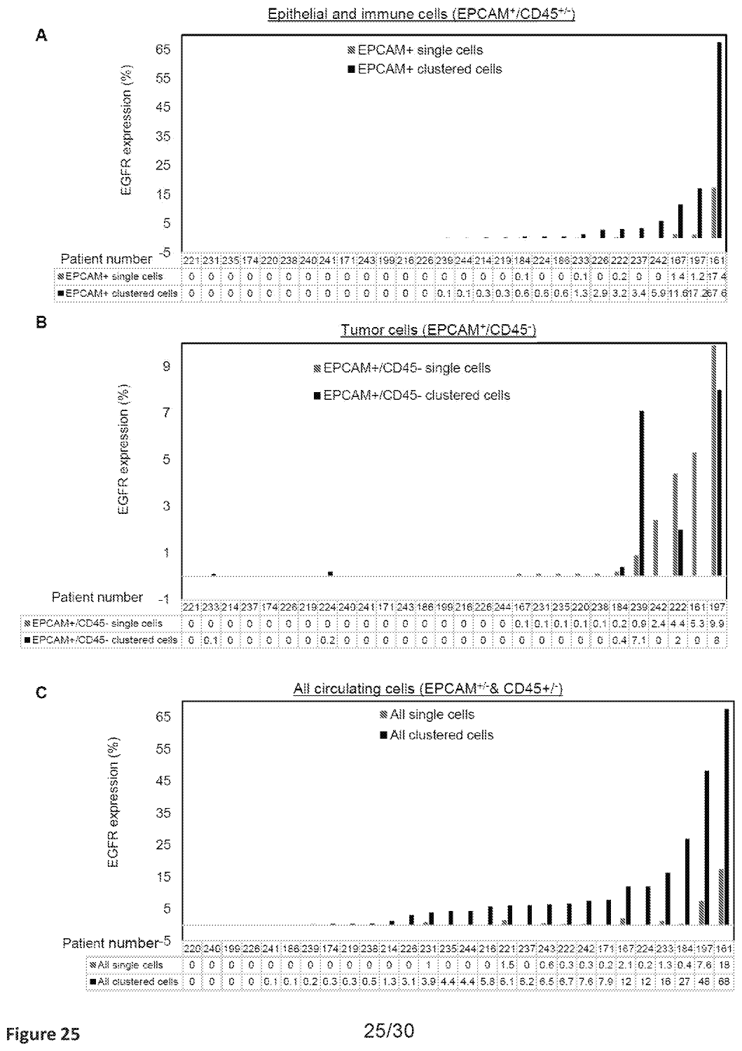

Inhibition of EGFR successfully blocks clustering and lung colonization. Using flow cytometry, we found enriched EGFR expression in clustered cells in the blood of breast cancer patients (FIG. 25). To determine the therapeutic effects of the EGFR blockade, we pre-treated the TN1 PDX cells overnight with anti-EGFR clone LA1 which blocked lung colonization of breast tumor cells in mice upon tail vein infusion (FIGS. 20A-C). Considering that our reduction in lung colonization could be contributed by the clustering formation we then decided to test if anti-EGFR would be effective in blocking lung colonization in vivo without pre-treatment. We did observe a reduction in lung colonization in mice treated with anti-EGFR (FIGS. 20D-F). Furthermore, tdTomato- and eGFP-labeled MDA-MB-231 cells were implanted orthotopically together into the mammary fat pads of mice for examination of tumor cell cluster-mediated polyclonal, spontaneous lung metastasis. When anti-EGFR was administered i.v. or i.p, and Erlotinib administered orally for 4 weeks to these mice, both had no effects on primary tumor formation, however significantly and dramatically reduced the spontaneous lung metastasis (FIGS. 21A-J). Notably, the dual color metastasis colonies observed in the lungs of the vehicle control mice were absent but with reduced single-colored colonies shown in Erlotinib treated mice (FIG. 19K). Consistently, CTC clusters in dual colors were only detectable in the blood collected from the vehicle-treated mice (FIG. 19L). This data shows that blocking EGFR receptor can successfully reduce cluster mediated polyclonal metastasis of breast cancer cells.

Methods

Cell culture and transfections. MDA-MB-231 cells were purchased commercially from ATCC, and verified to be mycoplasma-negative using Lonza's MycoAlert Mycoplasma Detection Kit. Cells were maintained in DMEM with 10% FBS+1% Penicillin-Streptomycin (P/S). Primary tumor cells were cultured in HuMEC ready medium (Life technologies)+5% FBS and 0.5% P/S, and Collagen type I (BD Biosciences) coated plates. MiRNAs (Dharmacon, negative control #4), and siRNAs (pooled) (Dharmacon, negative control A) were transfected using Dharmafect (Dharmacon) at 100 nM.

Western blot. Cells were lysed by RIPA buffer supplemented with Amresco protease inhibitor cocktail (1:100 diluted) and centrifuged for 10 mins at 4 degrees and 10,000 RPM. Protein concentration was measured and 20 ug of protein was loaded for each sample. Antibodies used: EGFR, p-EGFR, b-actin (abcam).

RNA extraction and real-time PCR. Total RNAs were extracted using Trizol (Invitrogen), and RNA was precipitated with isopropanol and glycogen (Invitrogen). After reverse transcription reactions, real-time PCR for miRNAs/genes were performed using individual miRNA/gene Taqman primers (Applied Biosystems) with ABI 7500 real time PCR system. RNU44 and U6 primers were used for miRNA internal controls and GAPDH for housekeeping gene control.

Mouse models and tumor dissociation. 8-10 weeks old NOD/SCIDmice were used for PDXs and human MDA-MB-231 cell-based xenograft studies. The triple negative PDXs and MDA-MB-231 cells were lentivirally labeled by eGFP, tdTomato, Luc2-eGFP (L2G), or Luc2-tdTomato (L2T) using the lentiviruses and labeling protocol as described previously³. PDX tumors were harvested and dissociated either with Collagenase III (TN1 model) or Liberase TH and TM research grade (TN2 model and lung tissues). Briefly, tumors were minced and incubated for 2-4 h at 37° C. with Collagenase III (Wortington Biomedical) or Liberase TH and TM (Roche) and 100 Kunitz U of DNase I (Sigma) in 20 mL of RPMI medium with 20 mM Hepes buffer. Single-cell suspensions were filtered through 40-μm nylon cell strainers and washed with Hanks' balanced saline solution (HBSS; Sigma) containing 2% heat-inactivated fetal bovine serum (FBS). Red blood cells were lysed with ACK lysis buffer, and dissociated bulk tumor cells were either cultured or stained with various antibodies in HBSS/2% FBS for further flow analysis or sorting on a BD FacsAria (BD Biosciences). DAPI and H2Kd were used as markers for viability and mouse stromal cells, respectively.

Bioluminescence imaging. Mice were injected intraperitoneally (i.p.) with 100 μL of D-luciferin (30 mg/mL, Gold biotechnology). After 5-10 mins, mice were anesthetized with isoflurane, and bioluminescence images were acquired using the Xenogen IVIS spectrum system (Caliper Life Sciences). Acquisition times ranged from 1 s-5 min. Signals are presented as total photon flux and analyzed using Living Image 3.0 software (Caliper Life Sciences).

Cell clustering assay. Freshly dissociated primary tumor cells in single cell suspension were seeded in Collagen type I-coated 96 well plates. The plates were put into the IncuCyte live cell imaging system (Essen BioScience), and live images were taken every 2-4 hours for up to one week. The cluster number and size were analyzed by Incucyte ZOOM software (Essen BioScience). In specific experiments, primary tumor cells might be sorted based on the expression of CD44 prior to seeding. In other experiments, seeded tumor cells might be transfected with siRNAs (100 nM) or treated with various inhibitors during the clustering assays. For cell viability analysis during clustering, the IncuCyte® Cytotox Red reagent (Essen BioScience) was added into the medium according to the instruction.

Statistical analysis. Student's T test was performed and probabilities under 0.05 were considered significant and represented with one star (*). Probabilities under 0.001 were represented with two stars (**).

### REFERENCES

1. Liu, X. et al. Homophilic CD44 Interactions Mediate Tumor Cell Aggregation and Polyclonal Metastasis in Patient-Derived Breast Cancer Models. Cancer Discov 9, 96-113, doi:10.1158/2159-8290.CD-18-0065 (2019).
2. Melo, F. S. et al. A distinct role for LgrS+ stem cells in primary and metastatic colon cancer. Nature 543, 676-680, doi:10.1038/nature21713 (2017).
3. Liu, H. et al. Cancer stem cells from human breast tumors are involved in spontaneous metastases in orthotopic mouse models. Proc Natl Acad Sci USA 107, 18115-18120, doi:10.1073/pnas.1006732107 (2010).
4. Adorno-Cruz, V. et al. Cancer stem cells: targeting the roots of cancer, seeds of metastasis, and sources of therapy resistance. Cancer Res 75, 924-929, doi:10.1158/0008-5472.CAN-14-3225 (2015).
5. Mani, S. A. et al. The epithelial-mesenchymal transition generates cells with properties of stem cells. Cell 133, 704-715, doi:10.1016/j.cell.2008.03.027 (2008).
6. Al-Hajj, M., Wicha, M. S., Benito-Hernandez, A., Morrison, S. J. & Clarke, M. F. Prospective identification of tumorigenic breast cancer cells. Proc Natl Acad Sci USA 100, 3983-3988, doi:10.1073/pnas.0530291100 (2003).
7. de Lau, W. et al. Lgr5 homologues associate with Wnt receptors and mediate R-spondin signalling. Nature 476, 293-297, doi:10.1038/nature10337 (2011).
8. Tian, H. et al. A reserve stem cell population in small intestine renders Lgr5-positive cells dispensable. Nature 478, 255-259, doi:10.1038/nature10408 (2011).
9. Gregorieff, A., Liu, Y., Inanlou, M. R., Khomchuk, Y. & Wrana, J. L. Yap-dependent reprogramming of Lgr5(+) stem cells drives intestinal regeneration and cancer. Nature 526, 715-718, doi:10.1038/nature15382 (2015).
10. Shimokawa, M. et al. Visualization and targeting of LGRS+ human colon cancer stem cells. Nature, doi:10.1038/nature22081 (2017).
11. Seshacharyulu, P. et al. Targeting the EGFR signaling pathway in cancer therapy. Expert Opin Ther Targets 16, 15-31, doi:10.1517/14728222.2011.648617 (2012).
12. Bockhorn, J. et al. MicroRNA-30c inhibits human breast tumour chemotherapy resistance by regulating TWF1 and IL-11. Nat Commun 4, 1393, doi:10.1038/ncomms2393 (2013).
13. Bockhorn, J. et al. MicroRNA-30c targets cytoskeleton genes involved in breast cancer cell invasion. Breast Cancer Res Treat 137, 373-382, doi:10.1007/s10549-012-2346-4 (2013).

### Example 3—ICAM1 as a New Therapeutic Target of Tumor Clusters in Cancer Metastasis

In search of new molecular targets that are responsible for mediating breast cancer metastasis, we identified ICAM1, intercellular adhesion molecule 1, highly enriched in the lung metastatic cells and circulating tumor cell clusters. ICAM1 directs intercellular homophilic interactions between tumor-tumor cells as well as tumor-endothelial cells. ICAM1 knockdown abolishes the tumor cell clustering and lung colonization of breast cancer cells. We further two anti-ICAM1 neutralizing antibodies (one polyclonal antibody, R&D Cat #AF720; and one mouse mAb IgG2a, anti-ICAM1 R6.5 from ATCC) that can block tumor clustering as well as transendothelial migration of breast cancer cells during metastasis.

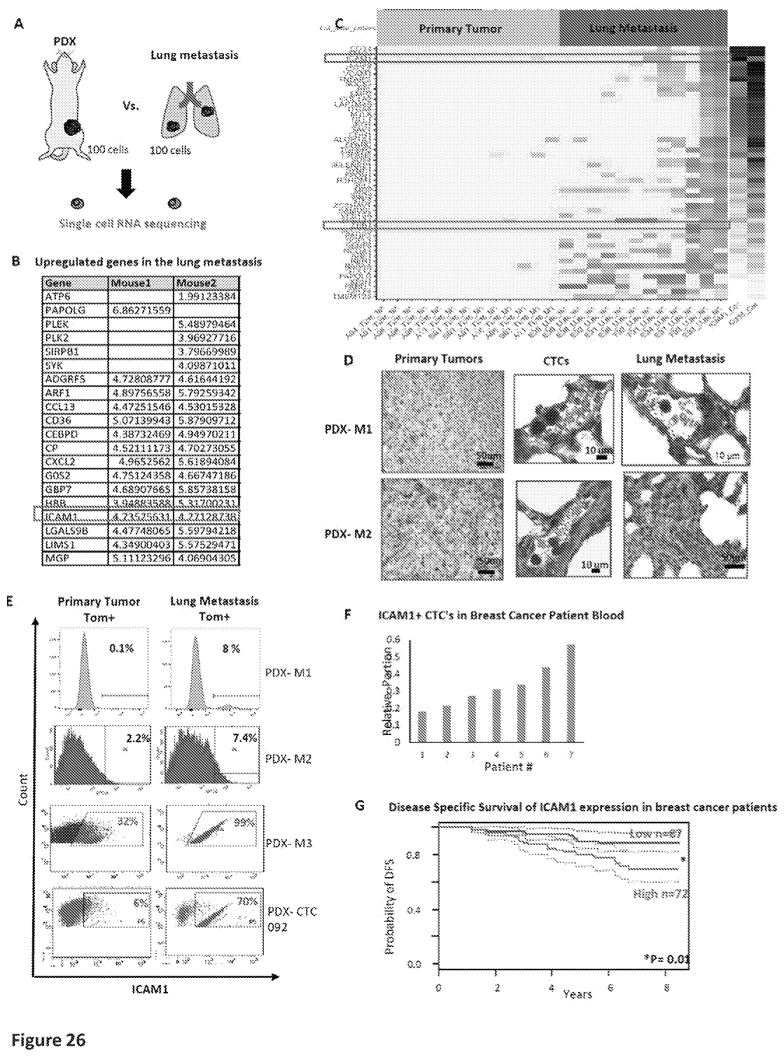

ICAM1 is highly expressed in lung metastatic triple negative breast cancer (TNBC) cells, and correlates with worse patient outcome. We have generated multiple triple negative breast cancer (TNBC) patient-derived-xenograft (PDX) mouse models, which spontaneously metastasize to the lungs [1]. To further determine the mechanisms of TNBC metastasis, we performed single cell RNA sequencing to compare transcriptomes of PDX primary tumor cells to that of lung metastatic cells (FIG. 26A). Among upregulated genes in the lung metastasis, ICAM1 expression positively correlated with other stemness signatures including ZEB1 (FIGS. 26B & 1C). Consistently, ICAM1 positive cells occupied a higher percentage of the lung metastatic cells compared to that of primary tumor cells; measured by IHC staining and flow cytometry analysis in multiple PDXs with spontaneous lung metastases (FIGS. 26D & 1E). Interestingly, ICAM1 was also highly expressed in circulating tumor cells in situ within the vasculature of PDX tissue sections (FIG. 26D). By CellSearch and flow cytometry analysis, ICAM1 was highly expressed in a good proportion of CTCs in patients (FIG. 26F). (Importantly, a high ICAM1 expression in breast tumors correlates with lower overall survival of breast cancer patients (FIG. 26G). Together, these data suggest that ICAM1 plays an important role in breast cancer metastasis.

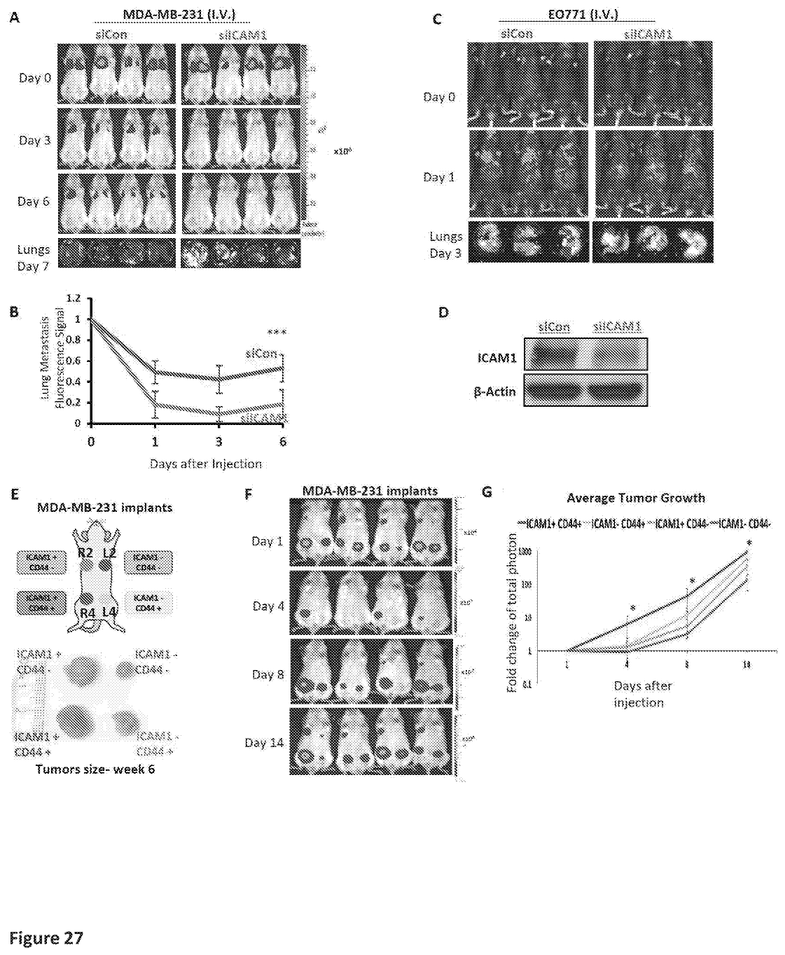

ICAM1 knockdown reduces metastatic and tumorigenic abilities of TNBCs. Next, we determined whether ICAM1 mediates metastasis in vivo. As expected, ICAM1 knockdown dramatically inhibited lung colonization of tail-vein-infused MDA-MB-231 and E0711 mouse tumor cells (FIG. 27A-D). In addition to lung colonization, we examined the tumor formation and growth of breast tumor cells with differential ICAM1 expression levels. Since CD44 is one of the well-known breast cancer stem cell markers, we compared tumorigenic ability among four cell populations with different CD44 and ICAM1 expression (ICAM1⁺CD44⁻, ICAM1⁻CD44⁺, ICAM1⁺CD44⁺ and ICAM1⁻CD44⁻). These cells were sorted from MDA-MB-231 cells, and then orthotopically injected (100 cells per mammary fat pad injection) into four different mammary fat pats of each mouse (FIG. 27E, n=4). After 14 days, the growth rate of ICAM1⁺CD44⁺ cells exceeded the other three populations (FIG. 27E-G), suggesting that ICAM1 further enhances the tumor growth of CD44⁺ cells.

ICAM1 mediates tumor cell clustering through homophilic interactions.

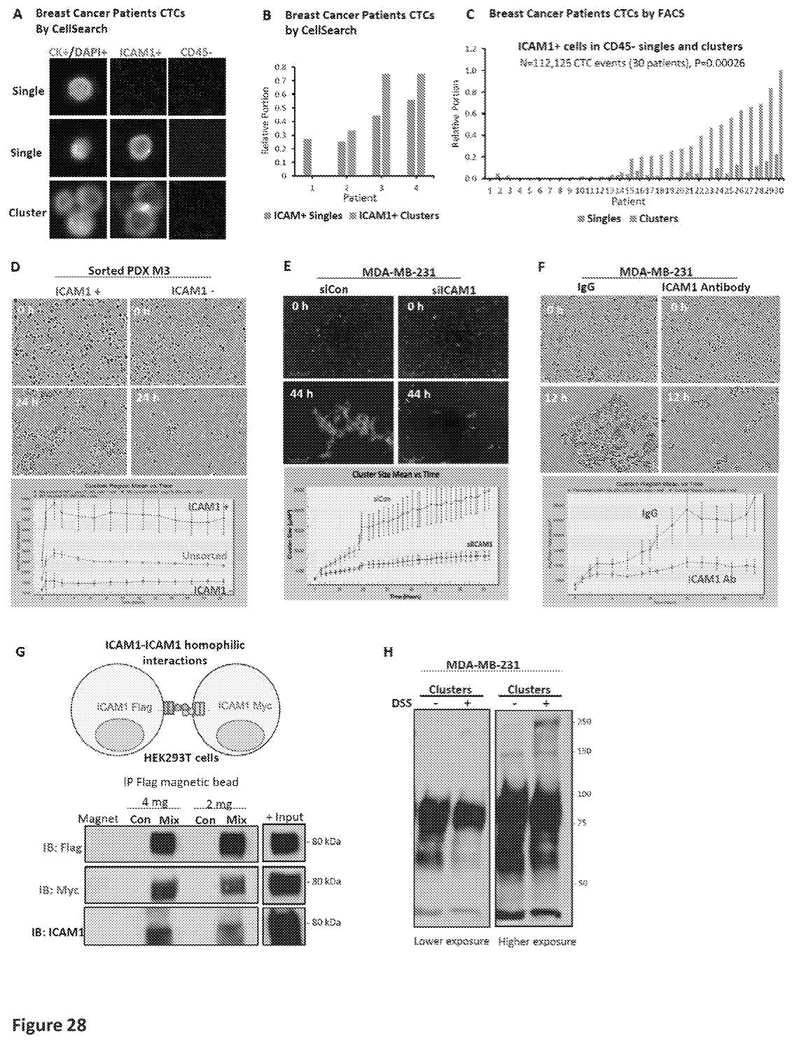

Recently, we found that CTC clusters enrich sternness for a higher metastatic potential compared to single CTCs [2]. To further understand the role of ICAM1 in metastasis, we measured the expression of ICAM1 on CTCs from breast cancer patients using the CellSearch platform as well as flow cytometry. Compared to single CTCs, the percentage of ICAM1⁺ cells increased in CTC clusters (FIG. 28A-C). Given ICAM1 is a well-known adhesion molecule; we continued to examine the importance of ICAM1 in PDX tumor cell clustering as described [2]. Compared to ICAM-1⁻ cells, sorted ICAM-1⁺ cells from PDX M3 model showed a higher efficiency forming bigger clusters (FIG. 28D). Secondly, ICAM-1 knockdown also decreased cluster formation of MDA-MB-231 cells (FIG. 28E). Consistently, ICAM1 neutralizing antibody dramatically inhibited cluster formation of MDA-MB-231 cells. Together, these data demonstrate that ICAM1 directs tumor cell clustering and serves a new therapeutic target of CTC clusters.

Next step was to determine how ICAM-1 mediates tumor cell clustering. We overexpressed ICAM1 with two different tags at its C-terminal (ICAM1-Flag and ICAM1-Myc) into two separate sets of HEK-293 cells. Upon dissociation, we mixed two sets of ICAM-Flag-expressing and ICAM1-Myc-expressing to form aggregated clusters (FIG. 27G, top) prior to collection for cell lysis and coimmunoprecipitation (co-IP). The results showed that ICAM1-Flag interacts with ICAM1-Myc from two neighboring cells, suggesting ICAM1 directs intercellular homophilic interactions. DSS crosslinking also revealed ICAM1 dimers (˜130 kDa) and possibly tetramers (˜260 kDa) in tumor clusters. ICAM1-ICAM1 homophilic interactions might be responsible for tumor cell clustering.

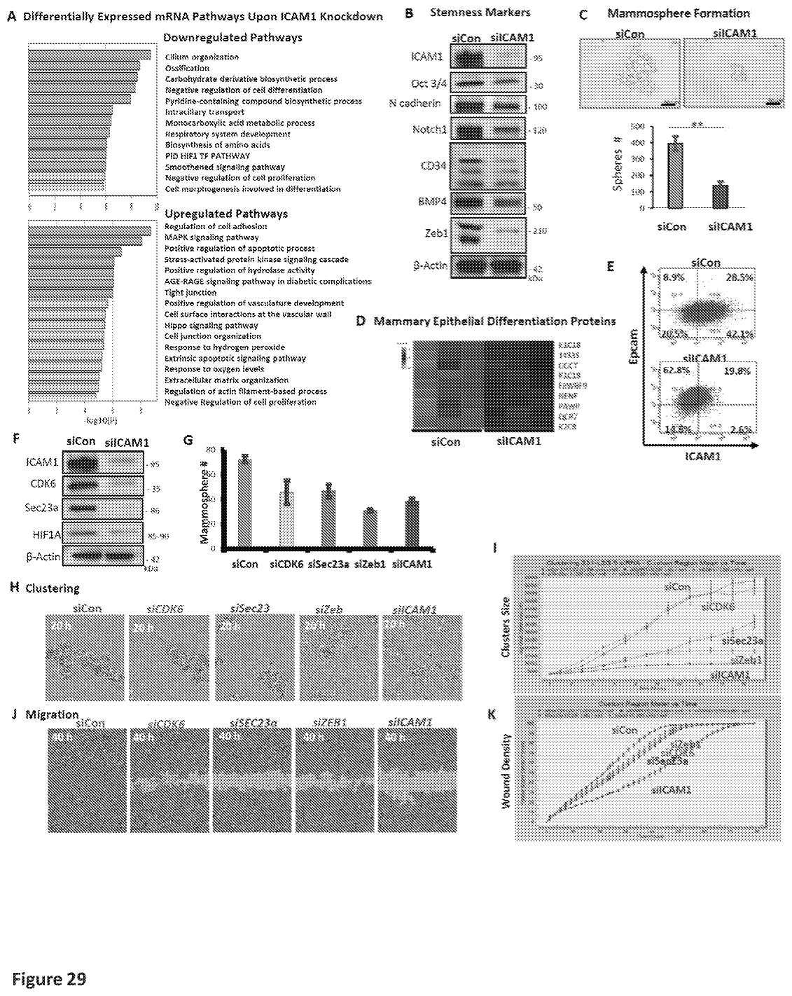

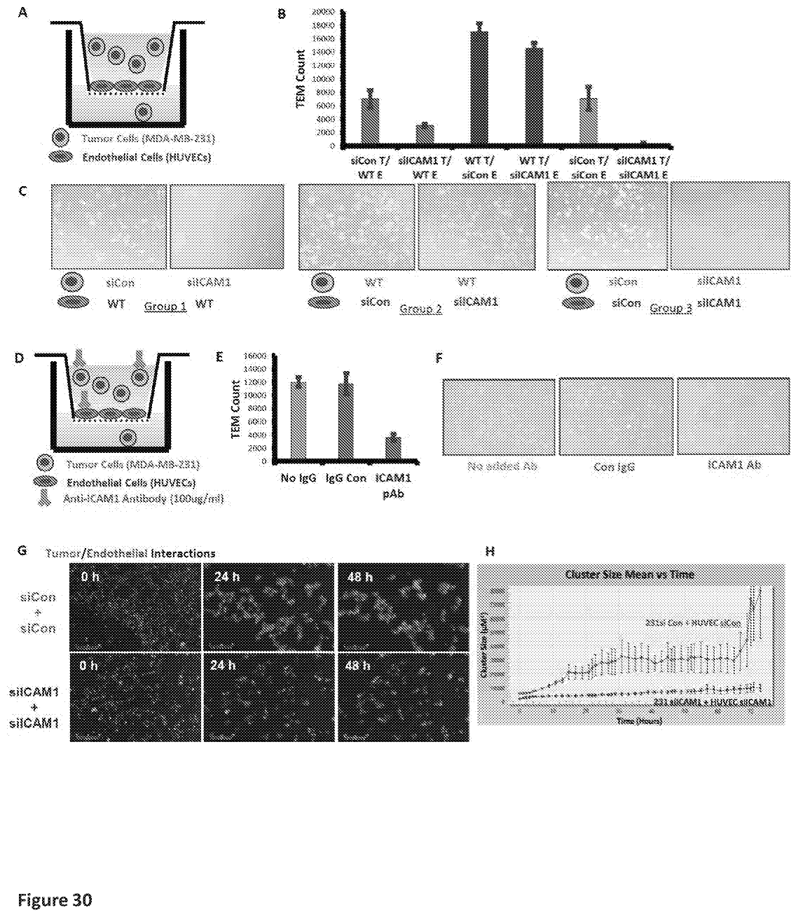

ICAM1 downstream pathways and target genes. To elucidate the downstream pathways, we compared the transcriptome and proteomics of MDA-MB-231 cells upon siICAM1-mediated knockdown. RNA sequencing revealed multiple downregulated pathways (biosynthesis, cell proliferation and sternness-related Smoothened signaling) as well as upregulated pathways (stress-activated signaling, cell junction, and apoptosis) (FIG. 29A). We confirmed a few ICAM1 targets and by western blotting, including N-Cadherin and BMP4, and Notch 1, CD34, Oct 3/4, ZEB1 (FIG. 29B). We then observed that upon ICAM1 knockdown, MDA-MB-231 breast tumor cells showed compromised mammosphere formation (FIG. 29C). Mass spectrometry-based proteomic analysis demonstrated an upregulation of mammary epithelial differentiation markers such as cytokeratin (CK) 18, CK19, and EpCAM in these siICAM1-transfected cells (FIG. 29D-E). Using immunoblotting, we further validate a few top targets of ICAM1 identified by both RNA seq and proteomic analyses, including CDK6, Sec23A and HIF1A (FIG. 30F). Knockdown of these targets and ZEB1 mimicked the inhibitory effects of ICAM1 knockdown on mammosphere formation, tumor clustering (Sec23A and ZEB1), and cell migration upon wound scratch (Sec23A, ZEB1, and CDK6) (FIG. 29G-K). However, these target genes had limited inhibitory effects on lung colonization of tail vein-infused MDA-MB-231 breast tumor cells (data not shown), we then examined the effects of ICAM1 on transendothelial migration which involves intercellular interactions with endothelial cells.

ICAM1 mediates transendothelial migration (TEM) of breast cancer cells. To metastasize, tumor cells have to transmigrate through the endothelium, where ICAM1 can be highly expressed. Since we found that ICAM-1 directs intercellular homophilic interactions, we hypothesize that ICAM1 enhances TEM through its heterotypic interactions between a tumor cell and an endothelial cell. To test this hypothesis, ICAM1 was knock downed in MDA-MB-231 cells, or HUVEC endothelial cells, or both. The TEM analyses demonstrated that knockdown ICAM1 in both cells completely inhibited TEM. Knockdown ICAM1 in either MDA-MB-231 cells, or HUVEC cells also dramatically inhibited TEM (FIG. 30A-C), suggesting that ICAM1 interactions are required for efficient TEM of tumor cells. To further confirm the role of ICAM1 in TEM, ICAM1 neutralizing antibody was added to the medium and shown to inhibited TEM of MDA-MB-231 cells (FIG. 30D-F). Additionally, knockdown ICAM1 in both MDA-MB-231 cells and HUVEC cells significantly reduced their direct interactions and cell aggregation (FIG. 30G-H). Taken together, these data suggest that inhibition of ICAM1 in tumor cells and endothelial cells significantly block TEM that is a rate-limiting step of metastasis.

### REFERENCES

1. Liu, H., et al., Cancer stem cells from human breast tumors are involved in spontaneous metastases in orthotopic mouse models. Proc Natl Acad Sci USA, 2010. 107(42): p. 18115-20.
2. Liu, X., et al., Homophilic CD44 Interactions Mediate Tumor Cell Aggregation and Polyclonal Metastasis in Patient-Derived Breast Cancer Models. Cancer Discov, 2019. 9(1): p. 96-113.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating cancer in a subject in need of treatment, the method comprising administered to the subject a therapeutic agent that inhibits aggregation of tumor cells.

2. The method of claim 1, wherein the cancer is characterized by circulating tumor cells (CTCs).

3. The method of claim 1, wherein the cancer is characterized by CTCs that express CD44, PAK2, EGFR, or ICAM1.

4. The method of claim 1, wherein the cancer is breast cancer.

5. The method of claim 1, wherein the cancer is estrogen receptor (ER)-negative breast cancer, the cancer is progesterone receptor (PR)-negative breast cancer, the cancer is human epidermal growth factor receptor 2 (HER2)-negative breast cancer, and/or the cancer is triple negative breast cancer (TNBC).

6. The method of claim 1, wherein the cancer is HER2-positive breast cancer.

7. The method of claim 1, wherein the therapeutic agent inhibits the biological activity of CD44.

8. The method of claim 1, wherein the therapeutic agent is an antibody or an antigen-binding fragment thereof that binds to CD44 and inhibits the biological activity of CD44.

9. The method of claim 1, wherein the therapeutic agent inhibits homophilic interactions between CD44 molecules present on the tumor cells.

10. The method of claim 1, wherein the therapeutic agent inhibits expression of CD44.

11. The method of claim 1, wherein the therapeutic agent inhibits the biological activity of protein activated kinase 2 (PAK2).

12. The method of claim 1, wherein the therapeutic agent inhibits the kinase activity of PAK2.

13. The method of claim 1, wherein the therapeutic agents is FRAX1036 (i.e., 6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one).

14. The method of claim 1, wherein the therapeutic target inhibits expression of PAK2.

15. The method of claim 1, wherein the therapeutic agent inhibits the biological activity of epidermal growth factor receptor (EGFR).

16. The method of claim 1, wherein the therapeutic agent is an antibody or an antigen-binding fragment thereof that binds to EGFR.

17. The method of claim 1, wherein the therapeutic agent inhibits the biological activity of intercellular adhesion molecule 1 (ICAM1).

18. The method of claim 1, wherein the therapeutic agent is an antibody or an antigen-binding fragment thereof that binds to ICAM1.

19. A method comprising detecting expression of one or more of CD44, PAK2, EGFR, and ICAM1 in circulating tumor cells of a subject having breast cancer.

20. The method of claim 19, further comprising identifying the subject as having a high risk for developing metastatic breast cancer.

21. The method of claim 19, further comprising administering to the subject a therapeutic agent that inhibits aggregation of tumor cells.

22. The method of claim 19, further comprising administering to the subject a therapeutic agent that inhibits the biological activity or expression of one or more of CD44, PAK2, EGFR, and ICAM1.
